(19) United States
(12) Patent Application Publication
(10) Pub. No.: US 2006/0052365 A1
(43) Pub. Date: Mar. 9, 2006

(54) Protease inhibitors

(76) Inventors: William E Bondinell, Wayne, PA (US); Ralph E Hall, King of Prussia, PA (US); Qi Jin, King of Prussia, PA (US); Jeffrey K Kerns, King of Prussia, PA (US); Hong Nie, King of Prussia, PA (US); Katherine L Widdowson, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/525,114

(22) PCT Filed: Aug. 22, 2003
(86) PCT No.: PCT/US03/26358

§ 371 Date: Sep. 27, 2005

Related U.S. Application Data
(60) Provisional application No. 60/405,227, filed on Aug. 22, 2002.

(51) Int. Cl. A61K 31/55; C07D 223/12; C07D 403/02

(52) U.S. Cl. 514/217.04; 540/527; 514/217.11

(57) ABSTRACT

This invention relates 4-amino-azepan-3-ones of formula (I) 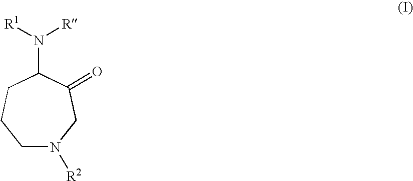 which are useful as protease inhibitors, particularly of cathepsin S, and as such are useful for preventing a number of diseases amongst which are atherosclerotic lesions and pulmonary diseases such as asthma and allergic reactions.

### FIELD OF THE INVENTION

This invention relates in general to the use of 4amino-azepan-3-one protease inhibitors, particularly such inhibitors of cathepsin S, in the treatment of diseases in which cathepsin S is implicated, especially treatment or prevention of autoimmune disease; treatment or prevention of a disease state caused by the formation of atherosclerotic lesions and complications arising therefrom; and diseases requiring inhibition, for therapy, of a class II MHC-restricted immune response, inhibition of an asthmatic response, inhibition of an allergic response, inhibition of immune response against a transplanted organ or tissue, or inhibition of elastase activity in atheroma; and novel compounds for treating same.

### BACKGROUND OF THE INVENTION

Cathepsins are a family of enzymes that are part of the papain superfamily of cysteine proteases. Cathepsins K, B, H, L, N and S have been described in the literature.

Cathepsins function in the normal physiological process of protein degradation in animals, including humans, e.g., in the degradation of connective tissue. However, elevated levels of these enzymes in the body can result in pathological conditions leading to disease. Thus, cathepsins have been implicated as causative agents in various disease states, including but not limited to, infections by pneumocystis carinii, trypsanoma cruzi, trypsanoma brucei brucei, and Crithidia fusiculata; as well as in schistosomiasis, malaria, tumor metastasis, metachromatic leukodystrophy, muscular dystrophy, amytrophy, and the like. See International Publication Number WO 94/04172, published on Mar. 3, 1994, and references cited therein. See also International Publication Number WO 97/16433 , published on May 9, 1997, and references cited therein.

Pathological levels of cathepsin S have been implicated in a variety of disease states. For instance, mice treated with inhibitor exhibited attenuated antibody response indicating that selective inhibition of cathepsin S may provide a therapeutic strategy for asthma and autoimmune disease processes. Thus, selective inhibition of cathepsin S may provide an effective treatment for diseases requiring, for therapy or prevention: inhibition of a class II MHC-restricted immune response; treatment and/or prevention of an autoimmune disease state such as rheumatoid arthritis, multiple sclerosis, juvenile-onset diabetes, sytemic lupus erythematosus, discoid lupus erythematosus, pemphigus vulgaris, pemphigoid, Grave's disease, myasthenia gravis, Hashimoto's thyroiditis, scleroderma, dermatomysositis, Addison's disease, pernicious anemia, primary myxoedema, thyrotoxicosis, autoimmune atrophic gastritis, stiff-man syndrome, Goodpasture's syndrome, sympathetic opthalamia, phacogenic uveitis, autoimmune haemolytic anaemia, idiopathic thrombocytopenic purpura, idiopathic leucopenia, primary biliary cirrhosis, active chronic hepatitis, cryptogenic cirrhosis, ulcerative colitis, Sjogren's syndrome, and mixed connective tissue diease; inhibition of an asthmatic response; inhibition of an allergic response; inhibition of immune response against transplanted organ or tissue inhibition of elastase activity in atheroma; and treatment or prevention of a disease state caused by the formation of atherosclerotic lesions or complications arising therefrom.

We have now discovered that certain 4-amino-azepan-3-one compounds inhibit cathepsin S, and are useful in the treatment of diseases in which cathepsin S is implicated.

### SUMMARY OF THE INVENTION

An object of this invention is the use of compounds of Formula I or II for inhibiting the activity of the protease inhibitors known as cathepsin S.

Another object of the present invention is to provide novel 4-amino-azepan-3-one carbonyl compounds of Formula II, as described below.

A further object of this invention is the use of a compound of Formula I or II in the manufacture of a medicament for treating or preventing a condition associated with the inhibition of cathepsin S.

Another aspect of this invention is that of a pharamceutical formulations comprising a compound of Formula II alone in admixture with a pharmaceutically acceptable excipient and administering this preparation to a mammal in need thereof in an amount effective for inhibiting cathepsin S to a degree which effects prevention of a condition or treatment of a condition associated with the inhibition of cathepsin S.

In a particular aspect, the methods of this invention are especially useful for treatment or prevention of autoimmune disease; treatment or prevention of a disease state caused by the formation of atherosclerotic lesions and complications arising therefrom; and diseases requiring inhibition, for therapy, of a class II MHC-restricted immune response, inhibition of an asthmatic response, inhibition of an allergic response, inhibition of immune response against a transplanted organ or tissue, or inhibition of elastase activity in atheroma.

### DETAILED DESCRIPTION OF THE INVENTION

The present invention provides a method for treating a disease by inhibiting cathepsin S comprising administering at least one compound of Formula I neat or as a pharmaceutically acceptable formulation, in an effective amount, wherein Formula I comprises: 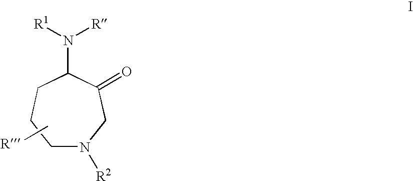
wherein:

R¹ is:

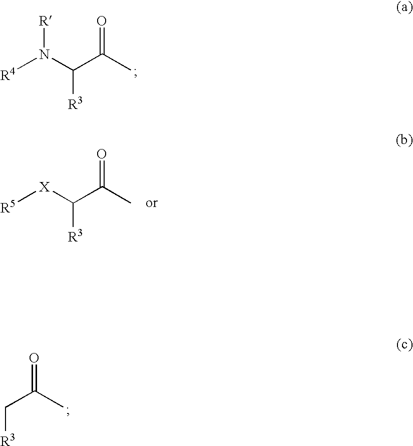

R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—

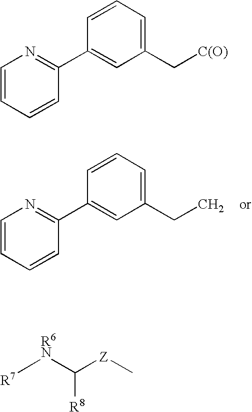

R³ is H or substituted or unsubstituted C₁₋₆alkyl, C₃₋₇cycloalkylC₀₋₆alkyl, C₄₋₇cycloalkenylC₀₋₆alkyl, C₅₋₈bicycloalkylC₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, or Het-HetC₀₋₆alkyl;

R³ and R′ may be connected to form a pyrrolidine, piperidine or morpholine ring;

R⁴ is H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵NSO₂—, R⁵OC(O)—, R⁵R¹³NC(O)—, or R⁵R¹³NC(S)—;

R⁵ is H, C₁₋₆alkyl, C₂₋₆alkenyl, C₂₋₆alkynyl, C₃₋₆cycloalkyl-C₀₋₆alkyl, Ar—C₀₋₆alkyl, Ar—ArC₀₋₆alkyl, Ar-HetC₀₋₆alkyl, Het-ArC₀₋₆alkyl, Het-HetC₀₋₆alkyl, or Het-C₀₋₆alkyl;

R⁶ is H, C₁₋₆alkyl, C₃₋₆cycloalkyl-C₀₋₆alkyl, Ar—C₀₋₆alkyl, or Het-C₀₋₆alkyl;

R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹⁴NC(O)—, or R¹⁰R¹⁴NC(S)—;

R⁸ is H, C₁₋₆alkyl, C₁₋₆alkenyl, C₂₋₆alkynyl, HetC₀₋₆alkyl or ArC₀₋₆alkyl;

R⁹ is C₁₋₆alkyl, C₃₋₆cycloalkyl-C₀₋₆alkyl, Ar—C₀₋₆alkyl or Het-C₀₋₆alkyl;

R¹⁰ is C₁₋₆alkyl, C₃₋₆cycloalkyl-C₀₋₆alkyl, Ar—C₀₋₆alkyl or Het-C₀₋₆alkyl;

R¹¹ is H, C₁₋₆alkyl, Ar—C₀₋₆alkyl, or Het-C₀₋₆alkyl;

R¹² is H, C₁₋₆alkyl, Ar—C₀₋₆alkyl, or Het-C₀₋₆alkyl;

R¹³ is H, C₁₋₆alkyl, Ar—C₀₋₆alkyl, or Het-C₀₋₆alkyl;

R¹⁴ is H, C₁₋₆alkyl, Ar—C₀₋₆alkyl, or Het-C₀₋₆alkyl;

R′ is H, C₁₋₆alkyl, Ar—C₀₋₆alkyl, or Het-C₀₋₆alkyl;

R″ is H, C₁₋₆alkyl, Ar—C₀₋₆alkyl, or Het-C₀₋₆alkyl;

R′″ is H, C₁₋₆alkyl, C₃₋₆cycloalkyl-C₀₋₆alkyl, Ar—C₀₋₆alkyl, or Het-C₀₋₆alkyl;

X is CH₂, S, or O;

Z is C(O) or CH₂; or

a pharmaceutically acceptable salt, hydrate or solvate thereof

This invention further provides the compounds of Formula II: 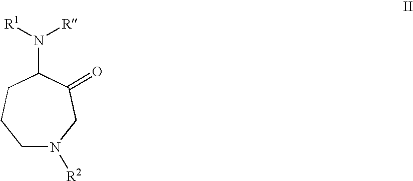
wherein:

R¹ is:

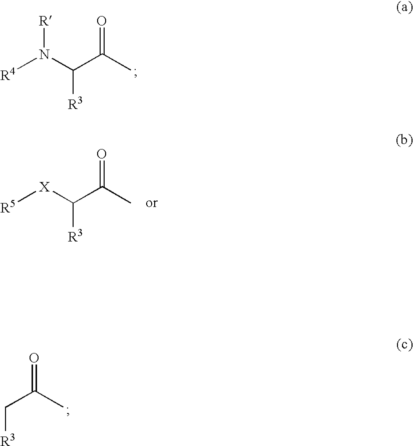

R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂—

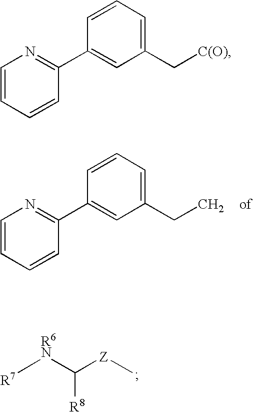

R³ is H or substituted or unsubstituted C₁₋₆alkyl, C₃₋₇cycloalkylC₀₋₆alkyl, C₄₋₇cycloalkenylC₀₋₆alkyl, C₅₋₈bicycloalkylC₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, or Het-HetC₀₋₆alkyl;

R⁴ is H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵NSO₂—, R⁵OC(O)—, R⁵R¹²NC(O)—, or R⁵R¹²NC(S)—;

R⁵ is H, C₁₋₆alkyl, C₂₋₆alkenyl, C₂₋₆alkynyl, C₃₋₆cycloalkyl-C₀₋₆alkyl, Ar—C₀₋₆alkyl, Ar—ArC₀₋₆alkyl, Ar-HetC₀₋₆alkyl, Het-ArC₀₋₆alkyl, Het-HetC₀₋₆alkyl, or Het-C₀₋₆alkyl;

R⁶ is H, C₁₋₆alkyl, C₃₋₆cycloalkyl-C₀₋₆alkyl, Ar—C₀₋₆alkyl, or Het-C₀₋₆alkyl;

R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, or R¹⁰R¹³NC(S)—;

R⁸ is H, C₁₋₆alkyl, C₂₋₆alkenyl, C₂₋₆alkynyl, HetC₀₋₆alkyl or ArC₀₋₆alkyl;

R⁹ is C₁₋₆alkyl, C₃₋₆cycloalkyl-C₀₋₆alkyl, Ar—C₀₋₆alkyl or Het-C₀₋₆alkyl;

R¹⁰ is C₁₋₆alkyl, C₃₋₆cycloalkyl-C₀₋₆alkyl, Ar—C₀₋₆alkyl or Het-C₀₋₆alkyl;

R¹¹ is H, C₁₋₆alkyl, Ar—C₀₋₆alkyl, or Het-C₀₋₆alkyl;

R¹² is H, C₁₋₆alkyl, Ar—C₀₋₆alkyl, or Het-C₀₋₆alkyl;

R¹³ is H, C₁₋₆alkyl, Ar—C₀₋₆alkyl, or Het-C₀₋₆alkyl;

R′ is H, C₁₋₆alkyl, Ar—C₀₋₆alkyl, or Het-C₀₋₆alkyl;

R″ is C₁₋₆alkyl, Ar—C₀₋₆alkyl, or Het-C₀₋₆alkyl;

X is CH₂, S, or O;

Z is C(O) or CH₂; or

a pharmaceutically acceptable salt, hydrate or solvate thereof.

In compounds of Formula I or II, R¹ is preferably group (a) 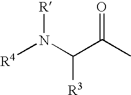
wherein:

R³ is preferably substituted or unsubstituted C₃₋₇cycloalkylC₀₋₆alkyl, C₄₋₇cycloalkenylC₀₋₆alkyl, C₅₋₈bicycloalkylC₀₋₆alkyl, Ar—ArC₀₋₆alkyl, Ar-HetC₀₋₆alkyl, Het-ArC₀₋₆alkyl, or Het-HetC₀₋₆alky.

More preferably R³ is substituted or unsubstituted C₅₋₇cycloalkylC₁₋₂alkyl, C₄₋₅cycloalkenylC₁₋₂alkyl, C₅₋₈bicycloalkylC₁₋₂alkyl or Ar-HetC₀₋₆alkyl.

Most preferably R³ is cyclopentylmethyl, cyclopentylethyl, cyclopentenylmethyl, cyclopentenylethyl, cyclohexylmethyl, 4-methylcyclohexylmethyl, 2-cyclohexylprop-1-yl, cyclohexylethyl, cycloheptylmethyl, 7,7-dimethylbicyclo[2.2.1]hept-1ylmethyl, or indol-2-ylmethyl;

R⁴ is R⁵C(O)— or R⁵SO₂— wherein R⁵ is C₃₋₆cycloalkyl-C₀₋₆alkyl, Ar—ArC₀₋₆alkyl, Ar-HetC₀₋₆alkyl, Het-ArC₀₋₆alkyl, or Het-HetC₀₋₆alkyl.

More preferably R⁵ is:

unsubstituted or substituted furanyl, especially furan-2-yl or furan-3-yl, or alkyl-substituted furanyl such as 2-methylfuran-3-yl, 2,4-dimethylfuran-3-yl, or aryl substituted furanyl, even more especially 5-phenylfuran-2-yl, 5-(2-chlorophenyl)furan-2-yl, 5-(3-chlorophenyl)furan-2-yl, 5-(4-chlorophenyl)furan-2-yl, 5-(4-fluorophenyl)furan-2-yl, 5-(4-hydroxyphenyl)furan-2-yl, 5-(3-trifluoromethylphenyl)furan-2-yl, 5-(4-trifluoromethylphenyl)furan-2-yl, 5-(3-trifluoromethylphenyl)furan-2-yl, 5-(4-methylphenyl)furan-2-yl, 5-(4-acetylphenyl)furan-2-yl, or 5-trifluoromethylfuran-2-yl;

unsubstituted or substituted tetrahydrofuranyl, particularly tetrahydrofuran-2-yl or tetrahydrofuran-3-yl

unsubstituted or substituted morpholinyl;

unsubstitutetd or substituted pyrrolyl, particularly pyrrol-2-yl;

unsubstituted or substituted piperazinyl, particularly piperzin-1-yl or 4-alkylpiperazinyl, e.g., 4methylpeperzin-1-yl;

unsubstituted or substituted pyrazolyl, particularly 1H-pyrazol-2-yl, 1H-pyrazol-4-yl, 1- or 2-methyl-2H-pyrazol-2-yl or 1- or 2-methyl-2H-pyrazol-3-yl;

unsubstituted or substituted isoxazolyl, particularly isoxazol-5-yl, 3-methylisoxazol-4-yl, 5-methylisoxazol-3-yl, 5-methylisoxazol-4-yl, or 3,5-dimethylisoxazol-4-yl;

unsubstituted or substituted thiazolyl, particularly thiazol-2-yl, 2-methylthiazol-2-yl, 2,4-dimethylthiazol-5-yl, 2-(2,3-dihydrobenzo[1,4]dioxin-2-yl)thiazol-4-yl, or 4-methyl-2-phenylthiazol-5-yl;

unsubsituted or C₁₋₂alkylsubstituted pyrazolo[5,1-c]triazinyl, particularly 4,7-dimethylpyrazolo[5,1-c]triazin-3-yl;

unsubstituted or substituted pyrazolyl, particularly alkyl-substituted pyrazolyl including 2-methyl-2H-pyrazol-2-yl;

C₁₋₂alkyl substituted pyrazolo[5,1-c]pyrimidinyl, particularly 2,7-dimethylpyrazol[5,1-c]pyrimidin-6-yl;

unsubstituted or aryl-substituted triazolyl, particularly phenyl-substituted triazoles including 3-phenyl-3H-{1,2,3]triazol-3-yl;

unsubstituted or substituted pyrazinyl, particularly pyrazin-2-yl and 5-methylpyrazin-2-yl;

unsubstituted or substituted imadazolyl, particularly 1-H-imidazol-2-yl, 1-methyl-1H-imidazol-4-yl or 1-methyl-1H-imidazol-2-yl;

benzofuranyl, especially benzofuran-2-yl, more especially C₁₋₆alkoxy substituted benzofuranyl, particularly 5,6-dimethoxybenzofuran-2-yl, more especially Het-C₀₋₆alkyl-benzofuran-2-yl, particularly 5-(2-morpholin-4-yl-ethoxy)benzofuran-2-yl;

thiophenyl, especially thiophene-3-yl and thiophen-2-yl, more especially Het-C₀₋₆alkylthiophenyl; particularly 5-pyridin-2-ylthiophen-2-yl, more especially C₁₋₆alkylthiophenyl, particularly 5-methylthiophen-yl or 3-methylthiophen-2-yl; more especially C₁₋₆alkoxythiophenyl, particularly 3-ethoxythiophen-2-yl;

furo[3,2-b]-pyridine-2-yl, especially 3-methylfuro[3,2-b]pyridin-2-yl;

phenyl, especially alkyl-substituted phenyl, halogen-substitutedphenyl, trihaloalkyl-substituted phenyl, alkoxy-substituted phenyl, or acetoxy-substitutedphenyl, especially 4-methylphenyl, 3-chlorophenyl, 4-chlorophenyl, 3-trifluoromethylphenyl, 4-trifluoromethylphenyl, 2-chlorophenyl, 4-fluorophenyl, 4-hydroxyphenyl, or 4-acetylphenyl;

unsubstituted or substituted pyridinyl, particularly pyridin-2-yl;

cyclobutyl or cyclopentyl;

unsubstituted or substituted, for example thieno[3,2-b]thiopheneyl, especially thieno[3,2-b]thiophen-2-yl or 5-isoxazol-3-ylthiophen-2-yl.

In group (a) R′ is preferably H, C₁₋₆alkyl, Ar—C₀₋₆alkyl, or Het-C₀₋₆alkyl, preferably H.

In compounds of Formula I and II, R² is preferably R⁹SO₂ or C₁₋₆alkyl. When R² is C₁₋₆alkyl, C₁₋₆alkyl is preferably propyl. R² is most preferably R⁹SO₂.

R⁹ is C₁₋₆alkyl, C₃₋₆cycloalkyl-C₀₋₆alkyl, Ar—C₀₋₆alkyl, or Het-C₀₋₆alkyl, preferably Het-C₀₋₆alkyl, more preferably pyridinyl or 1-oxy-pyridinyl. When R² is R⁹SO₂, R⁹ is even more preferably pyridin-2-yl or 1-oxy-pyridin-2-yl. Most preferably, R⁹ is pyridin-2-yl.

Most preferred compounds of Formula I or II are those wherein:

R¹ is group (a)

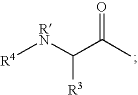

R² is R⁹SO₂;

R³ is cyclopentylmethyl, cyclopentylethyl, cyclopentenylmethyl, cyclopentenylethyl, cyclohexylmethyl, 4-methylcyclohexylmethyl, 2-cyclohexylprop-1-yl, cyclohexylethyl, cycloheptylmethyl, 7,7-dimethylbicyclo[2.2.1]hept-1-ylmethyl, or indol-2-ylmethyl;

R⁴ is R⁵C(O) or R⁵SO₂;

R⁵ is 5-phenylfuran-2-yl, 5-(2-chlorophenyl)furan-2-yl, 5-(3-chlorophenyl)furan-2-yl, 5-(4chlorophenyl)furan-2-yl, 5-(4-fluorophenyl)furan-2-yl, 5-(4-hydroxyphenyl)furan-2-yl, 5-(3-trifluoromethylphenyl)furan-2-yl, 5-(4-trifluoromethylphenyl)furan-2-yl, 5-(3-trifluoromethylphenyl)furan-2-yl, 5-(4-methylphenyl)furan-2-yl, 5-(4-acetylphenyl)furan-2-yl, or 5-trifluoromethylfuran-2-yl;

tetrahydrofuran-²-yl or tetrahydrofuran-3-yl

N-morpholinyl;

pyrrol-2-yl

piperzin-1-yl or 4-alkylpiperazinyl, e.g., 4-methylpeperzin-1-yl;

1H-pyrazol-2-yl, 1H-pyrazol-4-yl, 1- or 2-methyl-2H-pyrazol-2-yl or 1- or 2-methyl-2H-pyrazol-3-yl;

isoxazol-5-yl, 3-methylisoxazol-4-yl, 5-methylisoxazol-3-yl, 5-methylisoxazol-4-yl, or 3,5-dimethylisoxazol-4-yl;

thiazol-2-yl, 2-methylthiazol-2-yl, 2,4-dimethylthiazol-5-yl, 2-(2,3-dihydrobenzo[1,4]dioxin-2-yl)thiazol-4-yl, or 4-methyl-2-phenylthiazol-5-yl;

4,7-dimethylpyrazolo[5,1-]triazin-3-yl;

2-methyl-2H-pyrazol-2-yl;

2,7-dimethylpyrazol[5,1-]pyrimidin-6-yl;

3-phenyl-3H-{1,2,3]triazol-3-yl;

pyrazin-2-yl or 5-methylpyrazin-2-yl;

1-H-imidazol-2-yl, 1-methyl-1H-imidazol-4-yl or 1-methyl-1H-imidazol-2-yl;

benzofuran-2-yl, 5,6-dimethoxybenzofuran-2-yl, 5-(2-morpholin-4-yl-ethoxy)benzofuran-2-yl;

thiophene-3-yl, thiophen-2-yl, 5-pyridin-2-ylthiophen-2-yl, 5-methylthiophen-yl or 3-methylthiophen-2-yl, or 3-ethoxythiophen-2-yl;

furo[3,2-b]-pyridine-2-yl or 3-methylfuro[3,2-b]pyridin-2-yl;

phenyl, 4-methylphenyl, 3-chlorophenyl, 4-chlorophenyl, 3-trifluoromethylphenyl, 4-trifluoromethylphenyl, 2-chlorophenyl, 4-fluorophenyl, 4-hydroxyphenyl, or 4-acetylphenyl;

pyridin-2-yl;

thieno[3,2-b]thiophen-2-yl or 5-isoxazol-3-ylthiophen-2-yl

R⁹ is pyridin-2-yl or 1-oxy-pyridin-2-yl, preferably pyridin-2-yl;

R′ is H

R″ is H; and

in Formula I R′″ is C₁₋₆alkyl.

R′″ is preferably methyl, ethyl, propyl, butyl, pentyl and hexyl, more especially methyl; or preferably 5-, 6- or 7-C₁₋₆alkyl, especially 5-, 6- or 7-methyl, -ethyl, -propyl, -butyl, -pentyl or -hexyl, more especially 5-, 6- or 7-methyl; more preferably 6- or 7-C₁₋₆alkyl, especially 6- or 7-methyl, -ethyl, -propyl, -butyl, -pentyl and -hexyl, more especially 6- or 7-methyl; yet more preferably, in Formula I, cis-7-C₁₋₆alkyl as shown in Formula Ia: 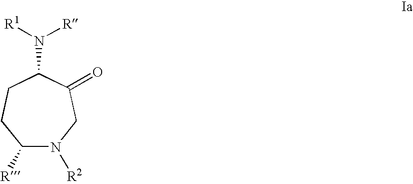
wherein R′″ is C₁₋₆alkyl, especially selected from the group consisting of: methyl, ethyl, propyl, butyl, pentyl and hexyl; most preferably cis-7-methyl, as shown in Formula Ia wherein R′″ is methyl.

As for the preferred substituents of Formula I, the definition are the same as those of the preferred compounds of Formula I with the exception of R³. For it the preferred groups are cyclopentylmethyl, [1-methylcyclopentyl]methyl, cyclopentylethyl, cyclopent-1-enylmethyl, cyclohexylmethyl, cycloheptylmethyl, [4-methylyclohexyl]methyl, [1-methylyclohexyl]methyl, and [2-7,7-dimethylbicyclo[2.2.1]hept-1-yl]ethyl. These preferred compounds, in particular, as well as other compounds of Formula II, are highly selective for inhibition of the cathepsin S enzyme as compared with their inhibition of the cathepsin K enzyme. Expressed as the ratio of the Kᵢ for cathepsin K over the Kᵢ of cathepsin S, (Kᵢ Cat K/Kᵢ Cat S) these novel compounds exhibit a ratio of 4 or greater in (define assay). The assay is described below.

Methods of Preparation

Compounds of the general formula Ia may be prepared in a fashion analogous to that outlined in Schemes 1 to 7. Carbobenyzloxy-D-alaninol (Cbz-D-alaninol)1 is first converted into an iodide and is then reacted with allyl Grignard with a copper (I) catalyst or a similar allyl organometallic reagent. The amine is then alkylated with allyl iodide. Grubbs' catalyst is then used to form the azepine ring 3 by ring closing metathesis. Epoxidation of the alkene followed by separation of the diastereomers and opening of the epoxide of the minor component with sodium azide provides the intermediate azido alcohol 5. Reduction of the azide 5 produces amine 6. 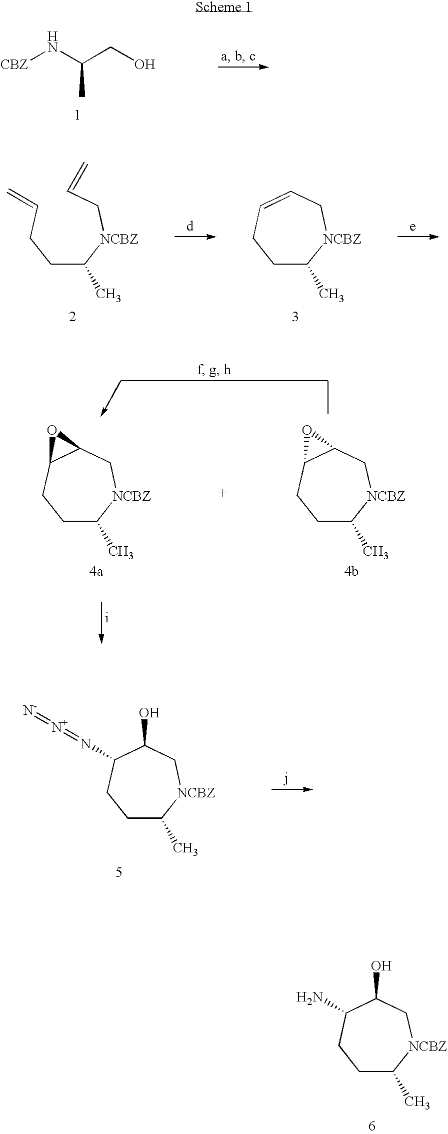
Reagents and conditions: (a) PPh₃, I₂; (b) 2-propenyl magnesium chloride, Cat. CuI; (c) allyl bromide, NaH; (d) Grubbs; (e) mCPBA; f) KOAc/HOAc, 18-crown-6; g) MeSO₂Cl, Et3N; h) KOH, MeOH; i) NaN₃; j) PPh₃

Commercially available methyl cyclopentane carboxylate was methylated with LDA and iodomethane to give 8 (scheme 2) Hydrolysis of the ester with LiOH followed by treatment with oxalyl chloride gives acid chloride 9. Subsequent Wolff rearrangement with diazomethane and silver benzoate produces ester 11. Reduction of the ester followed by oxidition with Dess-Martin periodinane produces aldehyde 13. This in turn is treated with KCN and (NH₄)CO₃ followed by hydrolysis with NaOH and protection of the free amine as its BOC carbamate to give amino acid 15.

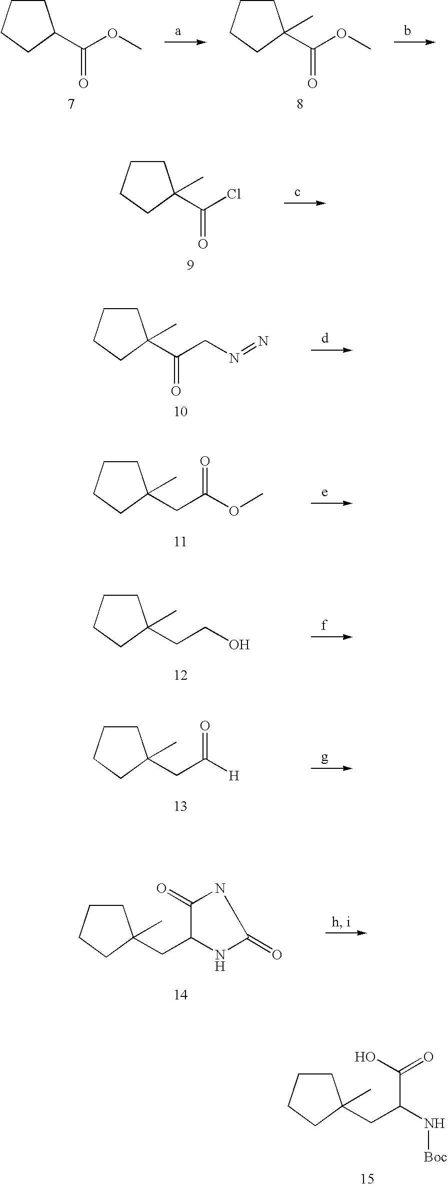

Reagents and conditions: (a) BuLi, diisopropylamine, MeI; b) LiOH, oxalylchloride; (c) CH₂N₂, Et₃N; (d) silver benzoate, Et₃N, MeOH; (e) LiAlH₄; f) Dess-Martin; g) KCN, (NH₄)₂CO₃, HCl; h) NaOH; i) (Boc)₂O.

The amine 6 may be protected with di-tert-butyldicarbonate to provide the N-Boc derivative 16 (Scheme 3). Removal of the benzyloxycarbonyl protecting group may be effected by treatment of 16 with hydrogen gas in the presence of a catalyst such as 10% Pd/C to provide the amine 17. Treatment of amine 17 with a sulfonyl chloride such as 2-pyridinesulfonyl chloride in the presence of a base such as N-methylmorpholine or triethylamine provides the sulfonamide derivative 18. Removal of the tert-butoxycarbonyl protecting group may be effected with an acid such as hydrochloric acid to provide intermediate 19. Coupling of 19 with an acid such as N-Boc-(1-methyl)cyclohexylalanine in the presence of a coupling agent common to the art such as HBTU or polymer supported EDC provides the alcohol intermediate 20. Removal of the tert-butoxycarbonyl protecting group under acidic conditions provides amine 21. Coupling of 21 with an acid such as furan-2-carboxylic acid in the presence of a coupling agent such as HBTU or polymer supported EDC provides alcohol 22. Alcohol 22 may be oxidized with an oxidant common to the art such as pyridine sulfur trioxide complex in DMSO and triethylamine or the Dess-Martin periodinane to provide the ketone 23.

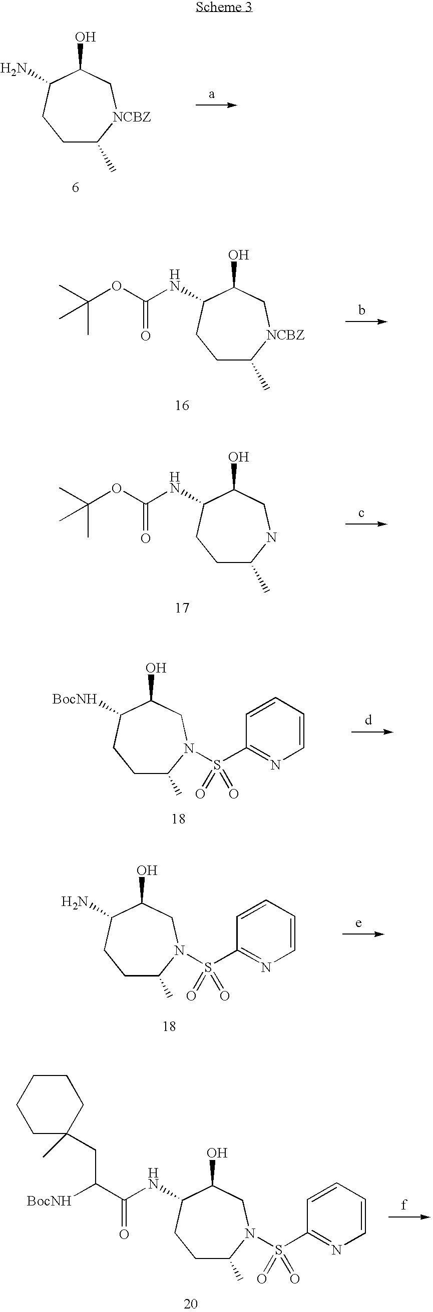

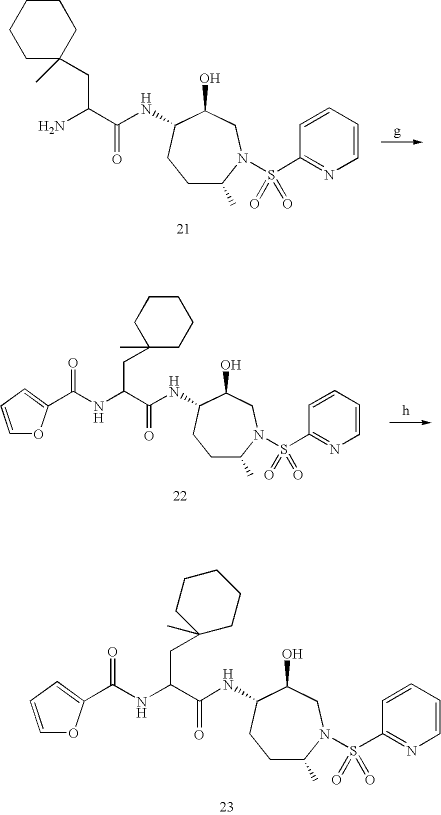

Reagents and conditions: (a) Di-tert-butyldicarbonate, THF; (b) H₂, 10% Pd/C, EtOAc; (c) 2-pyridinesulfonyl chloride, TEA, DMF; (d) HCl, MeOH; (e) N-Boc-1-methylcylohexylalanine, HBTU, 4-methylmorpholine, DMF; (f) HCl, MeOH; (g) furan-2-carboxylic acid, HBTU, 4-methylmorpholine, DMF; (h) Dess-Martin periodinane, methylene chloride.

The individual diastereomers of furan-2-carboxylic acid {(S)-2-[1-methylcyclohexyl-1-(4S,7R)-7-methyl-3-oxo-1-(1-oxy-pyridine-2-sulfonyl)-azepan-4-ylcarbamoyl]-ethyl}-amide 23a and 23b may be prepared as outlined in Scheme 4. The mixture of diastereomers are separated by HPLC to provide the compounds 23a and 23b. 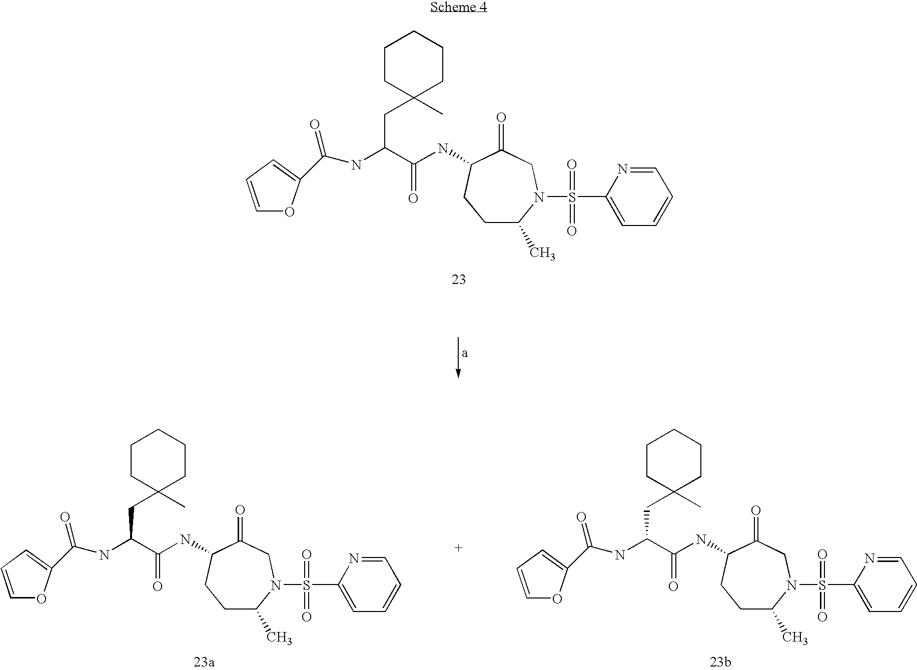
Reagents and Conditions: a.) HPLC separation.

Alternatively, the compounds of formula Ia may be prepared in a fashion analogous to Scheme 5. Thus, 1,4-pentadien-3-ol was epoxidized under Sharpless epoxidation conditions using cumene hydroperoxide and D-(−)-diisopropyl tartrate. The resulting secondary alcohol was inverted under Mitsonobu conditions with phthalimide to reveal epoxide 26. Opening of this epoxide with pyridine-2-sulfonic acid allylamide in the presence of DBU and subsequent ring-closing metathesis with Grubb's catalyst provided alkene 28. The olefin was hydrogenated over palladium on carbon and the phthalimide protecting group removed with hydrazine to reveal amine 30. The amine can then be used to couple to (S)-2-tert-Butoxycarbonylamino-3-cyclohexyl-propionic acid to provide intermediate 31. Subsequent removal of the tert-butoxycarbonyl protecting group, coupling with a carboxylic acid, and oxidation of the C3 secondary alcohol to the ketone provided 32. 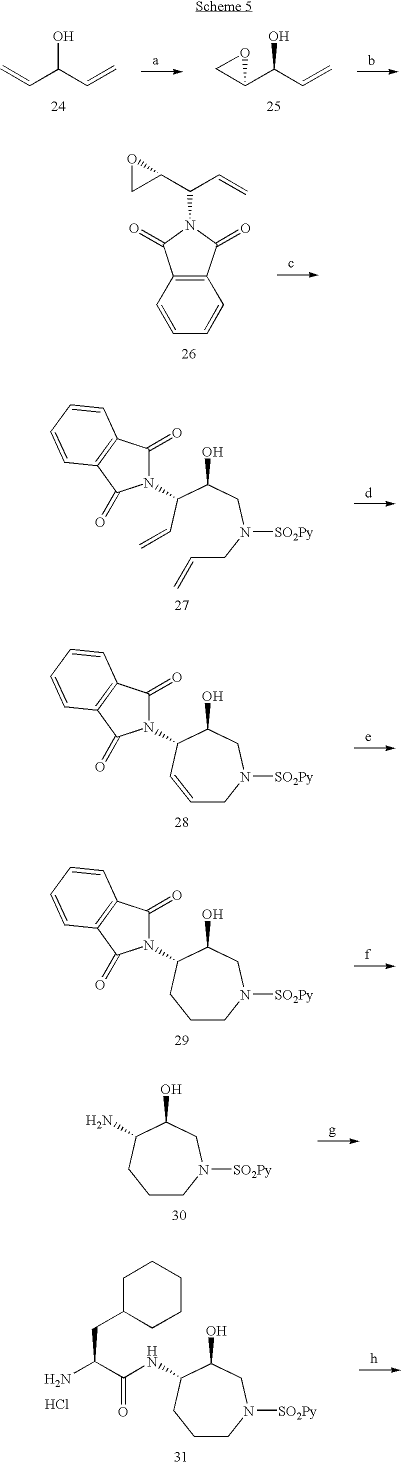 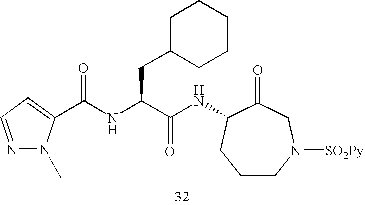
Reagents and conditions: a) Ti(OiPr)₄, cumene hydroperoxide, 4A molecular sieves, D-(−)-DIPT; b) phthalimide, Ph₃P,DIAD; c) Pyridine-2-sulfonic acid allylamide, DBU; d) Tricyclohexylphosphine(1,3-bistrimethylphenyl 4,5-dihydroimidazol-2-ylidene)benzylidene ruthenium (IV) dichloride; e) H₂(g), Pd/C, 45° C.; f) NH₂NH₂, MeOH, reflux; g) i) (S)-2-tert-butoxycarbonylamino-3-cyclohexyl-propionic acid, HBTU, 4-methylmorpholine; ii) 4N HCl; h) i) 2-methyl-2H-pyrazole-3-carboxylic acid, HBTU, 4-methylmorpholine; ii) Dess-Martin Periodinane

Compounds of the general formula Ia may also be prepared in a fashion analogous to that outlined in Schemes 6 to 7. Alkylation of benzyl-N-allylcarbamate (33) with a base such as sodium hydride and 5-bromo-1-pentene provides the diene 34 (Scheme 1). Treatment of 2 bis(tricyclohexylphosphine)benzylidine ruthenium (IV) dichloride olefin metathesis catalysts developed by Grubbs provides the tetrahydroazepine 35. Epoxidation of 35 with oxidizing agents common to the art such as m-CPBA provides the epoxide 36. Nucleophilic epoxide ring opening may be effected with a reagent such as sodium azide to provide the azido alcohol 37 which may be reduced to the amino alcohol 38 under conditions common to the art such as 1,3-propanedithiol and triethylamine in methanol or triphenylphosphine in THF and water. Treatment of amine 38 with S-Boc-cyclopentyl alanine in the presence of HBTU and 4-methylmorpholine affords compound 39. Removal of the tert-butoxycarbonyl protecting group may be effected by treatment of 39 with hydrogen chloride in dioxane to produce the amine 40. Treatment of amine 40 with 2-furoic acid in the presence of HBTU and 4-methylmorpholine produces compound 41. The benzyloxycarbonyl protecting group may be removed by treatment with TMSI in methylene chloride to provide amine 42. Treatment of amine 42 with a sulfonyl chloride such as 2-pyridinesulfonyl chloride in the presence of a base such as sodium bicarbonate gives secondary alcohol 43. Alcohol 43 may be oxidized with an oxidant common to the art such as pyridine sulfur trioxide complex in DMSO and triethylamine or the Dess-Martin periodinane to provide the ketone 44. 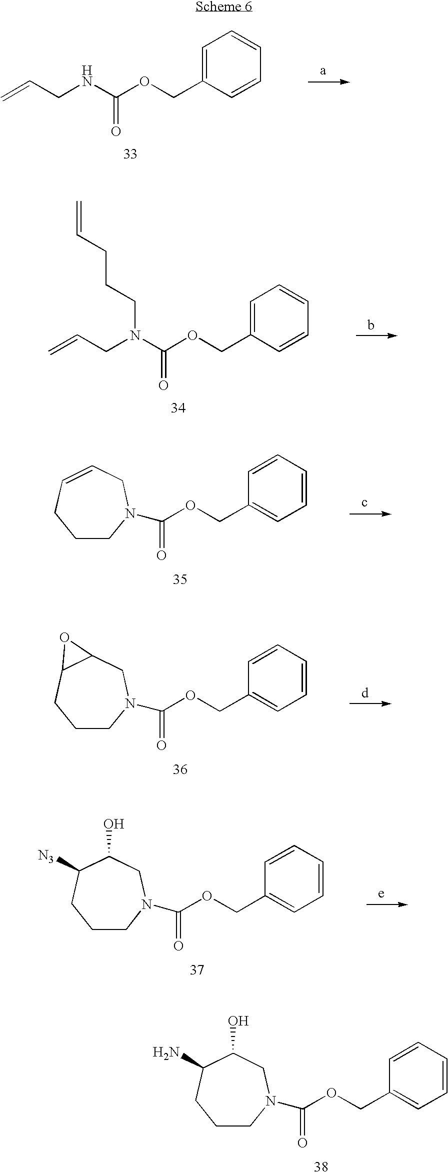
Reagents and conditions: (a) NaH, 5-bromo-1-pentene, NaH; (b) bis(tricyclohexylphosphine)benzylidine ruthenium (IV) dichloride, CH₂Cl₂, reflux; (c) m-CPBA, CH₂Cl₂; (d) NaN₃, NH₄Cl, CH₃OH, H₂O; (e) TEA, 1,3-propanedithiol, CH₃OH. 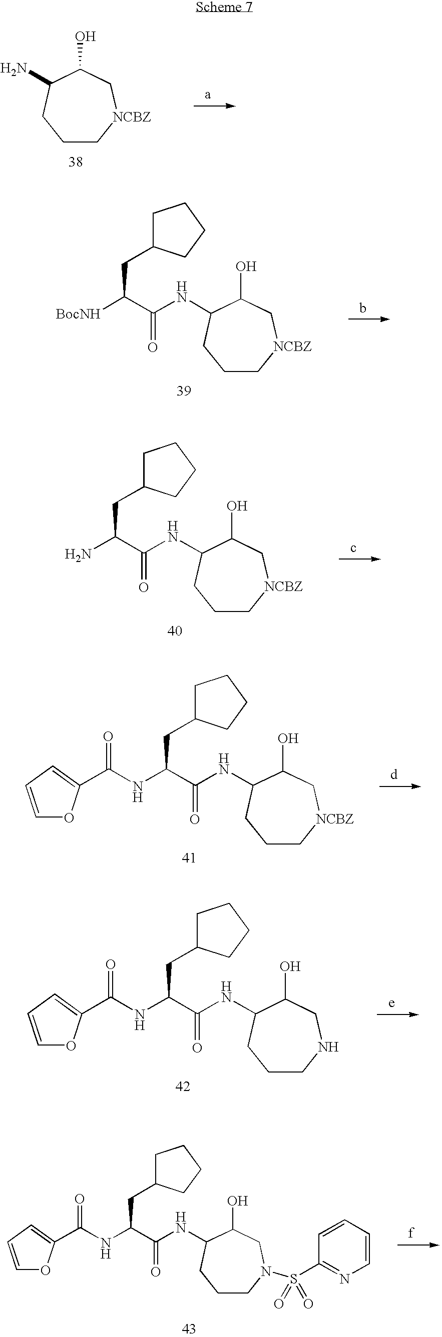 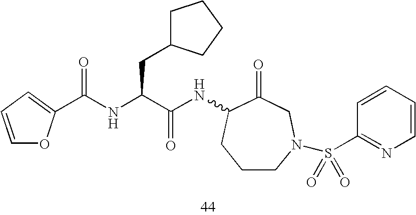
Reagents and conditions: (a) N-Boc-cylcopentylalanine, HBTU, 4-methylmorpholine, DMF; (b) HCl, dioxane; (c) 2-furoic acid, HBTU, 4-methylmorpholine, DMF; (d) TMSI, CH₂Cl₂; (e) 2-pyridyl sulfonylchloride, 10% sodium bicarbonate; (f) Dess-Martin periodinane, methylene chloride.

### UTILITY OF THE PRESENT INVENTION

The compounds of Formula I and II are useful as inhibitors of cathepsin S. The present invention provides methods of treatment of diseases caused by pathological levels of cathepsin S, which methods comprise administering to an animal, particularly a mammal, most particularly a human in need thereof a therapeutically effective amount of an inhibitor of cathepsin S, including a compound of the present invention.

The present invention particularly provides methods for treating the following diseases in which cathepsin S is implicated:

treatment and/or prevention of an autoimmune disease state such as rheumatoid arthritis, multiple sclerosis, juvenile-onset diabetes, systemic lupus erythematosus, discoid lupus erythematosus, pemphigus vulgaris, pemphigoid, Grave's disease, myasthenia gravis, Hashimoto's thyroiditis, scleroderma, dermatomysositis, Addison's disease, pernicious anemia, primary myxoedema, thyrotoxicosis, autoimmune atrophic gastritis, stiff-man syndrome, Goodpasture's syndrome, sympathetic opthalamia, phacogenic uveitis, autoimmune haemolytic anaemia, idiopathic thrombocytopenic purpura, idiopathic leucopenia, primary biliary cirrhosis, active chronic hepatitis, cryptogenic cirrhosis, ulcerative colitis, Sjogren's syndrome, and mixed connective tissue diease; and

treatment and/or prevention of a disease state caused by the formation and/or complications of atherosclerotic lesions.

Diseases which require therapy:

inhibition of a class II MHC-restricted immune response;

inhibition of an asthmatic response;

inhibition of an allergic response such as allergic rhinitis or atopic dermatitis;

inhibition of immune response against transplanted organ or tissue; and

inhibition of elastase activity in atheroma.

The present methods contemplate the use of one or more compounds of Formula I or II alone or in combination with other therapeutic agents.

For acute therapy, parenteral administration of a compound of Formula I or II is preferred. An intravenous infusion of the compound in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg, in a manner to maintain the concentration of drug in the plasma at a concentration effective to inhibit cathepsin S. The compounds are administered one to four times daily at a level to achieve a total daily dose of about 0.4 to about 400 mg/kg/day. The precise amount of a compound which is therapeutically effective, and the route by which such compound is best administered, is readily determined by one of ordinary skill in the art by comparing the blood level of the agent to the concentration required to have a therapeutic effect.

The compounds of Formula I or II may also be administered orally to the patient in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of Formula I or II are administered in accordance with the present methods.

Biological Assays

The compounds used in the present methods may be tested in one of several biological assays to determine the concentration of compound which is required to have a given pharmacological effect.

Determination of Cathepsin S Proteolytic Catalytic Activity

All assays for cathepsin S were carried out with human recombinant enzyme. Standard assay conditions for the determination of kinetic constants used a fluorogenic peptide substrate, typically Ac-Lys-Gln-Lys-Leu-Arg-AMC, and were determined in 50 mM Mes at pH 6.5 containing 10 mM cysteine and 5 mM EDTA. Stock substrate solutions were prepared at a concentration of 10 mM in 10% DMSO with 30 uM final substrate concentration in the assays. All assays contained 6% DMSO. All assays were conducted at 30° C. Product fluorescence (excitation at 360 nM; emission at 460 nM) was monitored either with a Perceptive Biosystems Cytofluor II fluorescent plate reader or a Tecan Spectraflour Plus plate reader. Product progress curves were generated over 20 to 30 minutes following formation of AMC product.

Determination of Cathepsin K Proteolytic Catalytic Activity

All assays for cathepsin K were carried out with human recombinant enzyme. Standard assay conditions for the determination of kinetic constants used a fluorogenic peptide substrate, typically Cbz-Phe-Arg-AMC, and were determined in 100 mM Na acetate at pH 5.5 containing 20 mM cysteine and 5 mM EDTA. Stock substrate solutions were prepared at a concentration of 10 mM in DMSO with 20 uM final substrate concentration in the assays. All assays contained 10% DMSO. All assays were conducted at 30° C. Product fluorescence (excitation at 360 nM; emission at 460 nM) was monitored either with a Perceptive Biosystems Cytofluor II fluorescent plate reader or a Tecan Spectraflour Plus plate reader. Product progress curves were generated over 20 to 30 minutes following formation of AMC product.

Determination of Cathepsin L Proteolytic Catalytic Activity

All assays for cathepsin L were carried out with human liver cathepsin L purchased from Enzyme Systems Products. Standard assay conditions are the same as cathepsin K except that the final substrate concentration was 5.0 uM.

Inhibition Studies

Potential inhibitors were evaluated using the progress curve method. Assays were carried out in the presence of variable concentrations of test compound. Reactions were initiated by addition of enzyme to buffered solutions of inhibitor and substrate. Data analysis was conducted according to one of two procedures depending on the appearance of the progress curves in the presence of inhibitors. For those compounds whose progress curves were linear, apparent inhibition constants (K_{i,app}) were calculated according to equation 1 (Brandt et al., Biochemitsry, 1989, 28, 140):
v=VₘA/[Kₐ(1+1/K_{i, app})+A] (1)
where v is the velocity of the reaction with maximal velocity Vₘ, A is the concentration of substrate with Michaelis constant of Kₐ, and I is the concentration of inhibitor.

For those compounds whose progress curves showed downward curvature characteristic of time-dependent inhibition, the data from individual sets was analyzed to give k_{obs} according to equation 2:
[AMC]=vₛₛt+(v₀-vₛₛ)[1−exp(−k_{obs}t)]/k_{obs} (2)
where [AMC] is the concentration of product formed over time t, v₀ is the initial reaction velocity and vₛₛ is the final steady state rate. Values for k_{obs} were then analyzed as a linear function of inhibitor concentration to generate an apparent second order rate constant (k_{obs}/inhibitor concentration or k_{obs}/[I]) describing the time-dependent inhibition. A complete discussion of this kinetic treatment has been fully described (Morrison et al., Adv. Enzymol. Relat. Areas Mol. Biol., 1988, 61, 201).
General

Nuclear magnetic resonance spectra were recorded at 400 MHz using, respectively, a Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel.

Where indicated, certain of the materials were purchased from the Aldrich Chemical Co., Milwaukee, Wis., Chemical Dynamics Corp., South Plainfield, N.J., and Advanced Chemtech, Louisville, Ky.

### EXAMPLES

In the following synthetic examples, temperature is in degrees Centigrade (° C). Unless otherwise indicated, all of the starting materials were obtained from commercial sources. Without further elaboration, it is believed that one skilled in the art can, using the preceding description, utilize the present invention to its fullest extent. These Examples are given to illustrate the invention, not to limit its scope. Reference is made to the claims for what is reserved to the inventors hereunder.

### Example 1

### Preparation of 1A: Morpholine 4-carboxylic acid {(S)-2-[1-methylcyclopentyl-1-(4S,7R)-7-methyl-3-oxo-1-(1-oxy-pyridine-2-sulfonyl)-azepan-4-ylcarbamoyl]-ethyl}-amide

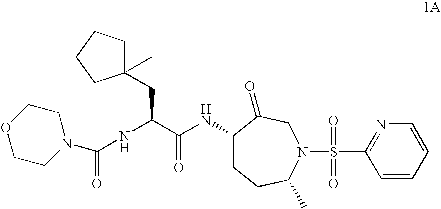

### Preparation of 1B: Morpholine 4-carboxylic acid {(L)-2-[1-methylcyclopentyl-1-(4S,7R)-7-methyl-3-oxo-1-(1-oxy-pyridine-2-sulfonyl)-azepan-4-ylcarbamoyl]-ethyl}-amide

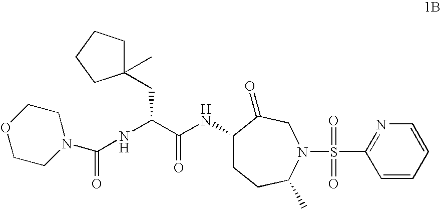

### 1a.) 1-Methyl methylcyclopentanecarboxylate

Butyllithium (1.6 M, 48.75 mL, 78 mmol) was added dropwise to a stirred solution of diisopropylamine (7.88 g, 44.5 mmol) in tetrahydrofuran (12 mL) at −78° C. The solution was warmed to room temperature to ensure the evaporation of butane and then cooled to −78° C. again. Methylcyclopentanecarboxylate (10.0 g, 78 mmol) in tetrahydrofuran (100 mL) was added to the reaction mixture at −78° C. After addition, the reaction mixture was warmed to 0° C. temperature for 30 mins. After cooling to −78° C., iodomethane (11.1 g, 78 mmol) in tetrahydrofuran (30 mL) was added. After addition, the reaction mixture was warmed to room temperature and stirred for 18 hours. Ammonium chloride solution (saturated) was added and the suspension was extracted with ether (3×). The combined organic phase was washed with water, brine, dried (MgSO₄), filtered and concentrated. Column chromatography (5% ethyl acetate:hexanes) of the residue provided 5.3 g of the title compound: ¹HNMR: (CDCL₃) δ 3.7 (s, 3H), 2.10 (s, 3H), 1.28-1.70 (m, 8H).

### 1b.) 1-Methylcyclopentanecarboxlic acid

To a solution of compound of Example 1a (5.3 g) in methanol was added lithium hydroxide (15.68 g, 0.4 mol). The reaction was stirred at room temperature for 18 hours. The reaction was concentrated in vacuo. The solution was adjusted to pH=1 with 10% HCl solution, and extracted with ethyl acetate. The combined organic phase was washed with water, brine, dried (MgSO₄), filtered and concentrated to give 5.0 g of the title compound: ¹HNMR: (CDCL₃) δ 2.10 (s, 3H), 1.26-1.73 (m, 8H).

### 1c.) 1-Methyl cyclopentanecarbonyl chloride

To a solution of the compound of Example 1b (5.0 g, 38.5 mmol) and oxalylchloride (3.6 mL) in CH₂Cl₂, 0.2 mL of DMF was added. The mixture was stirred overnight at room temperature. The solvent was removed under reduced pressure to give 5.0 g (crude) of the title compound which was used directly in the next step without further purification.

### 1d.) 1-Diazomethyl-1-methyl-cyclopentane

Triethylamine (6.12 mL, 43.94 mmol) was added to a solution of the 1-methyl cyclopentanecarbonyl chloride from Example 1c (5.0 g, 33.8 mmol) and diazomethane (1.47 g, 35 mmol) in a mixture of CH₃CN (25 mL) and THF (25 mL) at 0° C. After the addition was complete, the reaction mixture was allowed to warm room temperature for 20 hours. The solvent was removed under reduced pressure and the resulting residue washed with NaHCO₃ (sat.) solution and was extracted with ether (3×). The combined organic layers were washed with brine, dried (MgSO₄), filtered and concentrated to provide 4.0 g of the title compound: IR: N═N 2112.29 (cm⁻¹)

### 1e.) (1-Methyl-cyclopentyl)-acetic acid methyl ester

To a solution of the title compound of Example 1d (4.0 g, 25.8 mmol) in methanol (106 mL), 4 mL of silver benzoate (1.07 g) in triethyl amine (13.8 mL) was added. After addition, the reaction mixture was stirred at room temperature for 2 hours whereupon it was filtered to remove the solids. The filtrate was evaporated in vacuo. Column chromatography of the residue (20% ethyl acetate:hexane) provided 1.8 g of the title compound: ¹H NMR: (CDCl₃) δ 3.70 (s, 3H), 2.27 (s, 2H), 2.02 (s, 3H), 1.21-1.60 (m, 8H).

### 1f.) (1-Methyl-cyclopentyl)-ethanol

To a stirring solution of lithium aluminum hydride (24.73 mL, 23 mmol) in THF, the title compound of Example 1e (1.8 g, 11.5 mmol) was added slowly. After the addition, the mixture was stirred at reflux temperature for 2 hours after which time it was cooled to 0° C. Benzene (45 mL), water (1.77 mL) (added very slowly) and sodium fluoride (3.14 g) were added and stirred at 0° C. for 1 hour whereupon the suspension it was filtered to remove the solids. The filtrate was evaporated in vacuo to give the title compound (1.2 g).: ¹H NMR: (CDCl₃) δ 3.74 (m, 2H), 1.2-1.6 (m, 13H).

### 1g.) (1-Methyl-cyclopentyl)-acetaldehyde

To a solution of (1-methyl-cyclopentyl)-ethanol (Example 1f, 1.2 g, 9.37 mmol) in CH₂Cl₂ (20 mL), Dess-Martin periodinane (1.2 g) was added. After stirring for 2 hours, solutions of sodium thiosulfate (10% in water, 0.50 mL) and saturated aqueous sodium bicarbonate (0.50 mL) were added simultaneously to the reaction. The mixture was then extracted with ethyl acetate (2×). The organic layer was dried with MgSO₄, filtered, concentrated and purified via silica gel chromatography to give the title compound (1.1 g). ¹H NMR: (CDCl₃) δ 9.8 (s, 1H), 2.2 (s, 2H), 0.8-1.8 (m, 11H).

### 1h.) N-Boc-beta-(1-methylcyclopentyl)ala-OH

To a solution of (1-methyl-cyclopentyl)-acetaldehyde (Example 1g, 1.1 g, 8.73 mmol) in a mixture of ethanol (12 mL) and water (12 mL), potassium cyanide (624 mg, 9.6 mmol) and ammonium carbonate (2.26 g, 23.57 mmol) were added. The reaction mixture was stirred at 60° C. for 24 hours after which time the ethanol was removed in vacuo and the resultant aqueous solution was acidified to pH=1 with conc. HCl. The resultant white solid was collected by filtration, washed with water and dried under vacuum (420 mg). The product (420 mg) was refluxed in aqueous NaOH (aq.) (12 mL, 0.7 M) for 24 hours after which time the reaction mixture was concentrated to about 4 ml, and a solution of di-tert-butyldicarbonate 970 mg) in THF (10 mL) was added. After 2 hours, the THF was removed under vacuum, the residue was diluted with water (30 mL), and the mixture was washed with ether (2×). The aqueous phase was acidified to pH=1 with 1N aqueous HCl and then extracted with ethyl acetate (3×). The combined organic phase was washed with brine, dried, filtered, concentrated to give the title compound (300 mg). LC-MS m/z 271.2 (M⁺).

### 1i.) (1S,4R,7R)-4-Methyl-8-oxa-3-azabicyclo[5.1.0]octane-3-carboxylic acid benzyl ester

To a solution of (1R,4R,7S)-4-methyl-8-oxa-3-azabicyclo[5.1.0]octane-3-carboxylic acid benzyl ester (25 g, 95.4 mmol) in a mixture of toluene (210 mL) and DMSO (210 mL), potassium acetate (93.5 g, 954 mmol), acetic acid (5.72 g, 95.4 mmol) and 18-crown-6 (12.6 g, 47.7 mmol) were added at room temperature. The reaction mixture was stirred at 110° C. for 24 hours after which time the solvent was evaporated under reduced pressure. The residue was diluted with ethyl acetate and was washed with water, sodium bicarbonate (sat.) and brine. The combined organic layer was dried over MgSO₄, filtered and concentrated under reduced pressure to give a mixture of products which was used directly in the next step without further purification (27.86 g). LC-MS m/z 322.0 (M⁺).

To solution of the mixture compounds (from above) (27.86 g, 86.8 mmol) in methylene chloride (400 mL), methanesulfonyl chloride (10.12 ml, 130.2 mmol) and triethylamine (24.2 mL, 173.6 mmol) were added. The reaction mixture was stirred at room temperature for 5 hours. It was then partitioned between methylene chloride and water. The combined organic layer was dried over MgSO₄, filtered and concentrated under reduced pressure to give a mixture of products which were used directly in the next step without further purification (30.5 g). LC-MS m/z 400.0 (M⁺).

To a solution of the mixture of compounds (from above) (30.5 g, 76.2 mmol) in methanol (100 mL), 10% potassium hydroxide solution (100 mL) was added at room temperature. The reaction mixture was stirred at room temperature for 24 hours, after which time the solvent was removed under reduce pressure. The residue was partitioned between ethyl acetate and water. The combined organic layer was dried over MgSO₄, filtered and concentrated under reduced pressure to give a mixture of products. Silica gel chromatography of the mixture of epoxides (20% Ethyl acetate/80% Hexane) gave the title compound (7.47 g) and undesired epoxide product (10.5 g). LC-MS m/z 262 (M⁺).

### 1j.) (2R,5S,6S)-5-Azido-6-hydroxy-2-methyl-azepane-1-carboxylic acid benzyl ester

A 1-liter round bottom flask was charged with (1S,4R,7R)-4-methyl-8-oxa-3-azabicyclo[5.1.0]octane-3-carboxylic acid benzyl ester (Example 1i, 7.47 g, 28.3 mmol). Ethylene glycol (46 ml) was then added. Triethanolamine (23.7 ml, 169.8 mmol) was dissolved in H₂O (46 ml), then was added. NH₄Cl (4.54 g, 84.9 mmol), then sodium azide (5.52 g, 84.9 mmol) was added and the reaction was stirred behind a blast shield at 80° C. overnight. The reaction mixture was cooled to RT, then poured into 10% aqueous NaCl. The mixture was extracted with CH₂Cl₂, and the combined organics were back extracted with aqueous NaHCO₃, then brine, dried with MgSO₄, filtered, concentrated in vacuo, and purified by flash column chromatography (20% to 33% ethyl acetatelhexanes, silica gel) to yield the title compound (7.4 g, 86%). LC-MS m/z 305.0 (M⁺).

### 1k.) (2R,5S,6S)-5-Amino-6-hydroxy-2-methyl-azepane-1-carboxylic acid benzyl ester, HCl salt

(2R,5S,6S)-5-Azido-6-hydroxy-2-methyl-azepane-1-carboxylic acid benzyl ester (Example 1j, 6.6 g, 21.7 mmol) was dissolved in THF (100 ml) and H₂O (2.8 ml), then triphenylphosphine (8.5 g, 32.6 mmol) was added and the reaction was stirred at R.T overnight. The reaction mixture was concentrated in vacuo, and the remaining solid dissolved in MeOH (10 ml). 1 M HCl in Et₂O (20 ml) was added, then the solution was concentrated in vacuo to a solid. This was dissolved in a minimum amount of MeOH in a round bottle flask and the solution triturated with Et₂O (˜500 mL) to precipitate triphenylphospine oxide. The solid was removed via filtration and the above procedure repeated several times until no UV active component was being further extracted (<10% UV absorption of triphenylphospine oxide by LC-MS). The remaining solid was used in the next reaction without further purification (6.6 g, 91%). LC-MS m/z 279.2 (M⁺).

### 1l.) (2R,5S,6S)-5-N-Bocamino-6-hydroxy-2-methyl-azepane-1-carboxylic acid benzyl ester

To a solution of (2R,5S,6S)-5-amino-6-hydroxy-2-methyl-azepane-1-carboxylic acid benzyl ester, HCl salt (Example 1k, 6.91 g, 22 mmol) in dioxane (74 mL), sodium hydroxide (1.76 g, 44 mmol) and water (13 mL) were added. Then the reaction mixture was cooled to 0° C. Di-tert-butyl dicarbonate (5.28 g, 24.2 mmol) was added, and the reaction mixture was allowed to warm to room temperature for 16 hours. The solvent was evaporated, and the residue was diluted with ethyl acetate and washed with H₂O, 10% HCl solution, NaHCO₃(aq.) and brine. The combined organic layer was dried over MgSO₄, filtered and concentrated under reduced pressure to give a crude product. Chromatography of the resulting solid on silica gel (30% Ethyl acetate/70% Hexane) gave the title compound (7.94 g, 95%). LC-MS m/z 379.2 (M⁺).

### 1m.) [(3S,4S,7R)-3-Hydroxy-7-methyl-azepan-4-yl]-carbamic acid tert-butyl ester

To a solution of (2R,5S,6S)-5-N-bocamino-6-hydroxy-2-methyl-azepane-1-carboxylic acid benzyl ester (Example 1l, 7.94 g, 20.9 mmol) in ethanol (200 mL), palladium (10 wt. % on activated carbon) (1.7 g) was added. The reaction mixture was hydrogenated at 45 psi for 5 hours. The reaction mixture was filtered through celite, concentrated in vacuo by rotary evaporation to give the title compound which was used without further purification (5.0 g, 97%). LC-MS m/z 245.0 (M⁺).

### 1n.) [(3S,4S,7R)-3-Hydroxy-7-methyl-1-(pyridine-2-sulfonyl)-azepan-4-yl]-carbamic acid tert-butyl ester

A solution of 2-mercaptopyridine (10 g, 90 mmol) in a mixture of conc. HCl (116 mL) and H₂O (34 mL) was cooled to 0° C. Chlorine gas was bubbled into the solution at 0° C. for 3.0 hours. Ice was added to the reaction mixture, followed by extraction with cold ether (2×). The ether layer was washed with cold 10% NaHCO₃ solution, and cold brine. The ether layer was dried over MgSO₄, filtered and concentrated under reduced pressure to give 2-pyridine sulfonyl chloride which was used without further purification (12.86 g, 80%). LC-MS m/z 178.0 (M⁺).

Triethyl amine (9.38 mL, 67.32 mmol) was added to a solution of [(3S,4S,7R)-3-hydroxy-7-methyl-azepan-4-yl]-carbamic acid tert-butyl ester (Example 1m, 5.0 g, 20.4 mmol) in methylene chloride (50 mL). The reaction mixture was cooled to 0° C., whereupon a solution of 2-pyridine sulfonyl chloride (3.26 g, 18.36 mmol) in methylene chloride (10 mL) was added dropwise. The resulting solution was stirred at room temperature for 4 hours. The reaction mixture was partitioned between methylene chloride and water. The aqueous phase extracted further with methylene chloride. The combined organic layer was dried over MgSO₄, filtered and concentrated under reduced pressure to give a crude product. Chromatography of the resulting solid on silica gel (70% Ethyl acetate/30% Hexane) gave the desired product (5.6 g, 71%). LC-MS m/z 386.0 (M⁺).

### 1o.) (3S,4S,7R)-3-Hydroxy-7-methyl-1-(pyridine-2-sulfonyl)-azepan-3-ol, HCl salt

HCl in dioxane (4.0 M, 89 mL) was added to a stirred solution of [(3S,4S,7R)-3-hydroxy-7-methyl-1-(pyridine-2-sulfonyl)-azepan-4-yl]-carbamic acid tert-butyl ester (Example 1n, 5.6 g, 14.5 mL) in MeOH (30 mL). The reaction mixture was stirred for 2 hours at room temperature, then concentrated in vacuo to yield a white solid. This was used in the next reaction reaction without further purification (5.7 g). LC-MS m/z 286.0 (M⁺).

### 1p.) 2-Amino-N-[(3S,4S,7R)-3-hydroxy-7-methyl-1-(pyridine-2-sulfonyl)-azepan-4-yl]-3-(1-methyl-cyclopentyl)-propionamide, HCl salt

To a solution of (3S,4S,7R)-4-amino-7-methyl-1-(pyridine-2-sulfonyl)-azepan-3-ol, HCl salt (Example 1o, 358 mg, 1.11 mmol) in DMF, N-Boc-beta-(1-methylcyclopentyl)ala-OH (Example 1h, 300 mg, 1.11 mmol), HBTU (547 mg, 1.47 mmol) and 4-methylmorpholine (561 mg, 5.55 mmol) were added. After the reaction mixture was stirred at room temperature for 16 hours, it was partitioned between ethyl acetate and water. The combined organic phase was washed with water, brine, dried (MgSO₄), filtered and concentrated. Column chromatography (5% methanol:CH₂Cl₂) of the residue provided the N-Boc title compound (220 mg, 37%). MS (m/z) 539.0 (M⁺).

To a stirring solution of N-Boc-2-amino-N-[(3S,4S,7R)-3-hydroxy-7-methyl-1-(pyridine-2-sulfonyl)-azepan-4-yl]-3-(1-methyl-cyclopentyl)-propionamide (220 mg, 0.41 mmol) in methanol (1 mL) was added HCl (4M in dioxane) (2.54 mL). After stirring at room temperature for 2 hours, the mixture was concentrated, giving a white solid. The white solid was azeotroped with toluene (2×) and then concentrated to give the title compound as a solid (200 mg). MS (m/z) 439.0 (M⁺).

### 1q.) Morpholine-4-carboxylic acid [1-[(3S,4S,7R)-3-hydroxy-7-methyl-1-(pyridine sulfonyl)-azepan-4-ylcarbamoyl]-2-(1-methyl-cyclopentyl)-ethyl]-amide

To a stirring solution of 2-amino-N-[(3S,4S,7R)-3-hydroxy-7-methyl-1-(pyridine-2-sulfonyl)-azepan-4-yl]-3-1-methylcyclopentyl)-propionamide (Example 1p, 0.2 g, 0.46 mmol) in CH₂Cl₂ (5 mL) were added 4-morpholinecarbonyl chloride (69 mg, 0.46 mmol) and triethyl amine (0.384 ml, 2.76 mmol). After stirring at room temperature for 16 hours, the reaction mixture was washed with water, brine, dried (MgSO₄), filtered and concentrated. Column chromatography (5% methanol:CH₂Cl₂) of the residue provided the title compound (120 mg, 47%). MS m/z 552.2 (M⁺).

### 1r.) Morpholine 4-carboxylic acid {(S)-2-[1-methylcyclopentyl-1-(4S,7R)-7-methyl-3-oxo-1-(1-oxy-pyridine-2-sulfonyl)-azepan-4-ylcarbamoyl]-ethyl}-amide

To a stirring solution of morpholine-4-carboxylic acid [1-[(3S,4S,7R)-3-hydroxy-7-methyl-1-(pyridine-2-sulfonyl)-azepan-4-ylcarbamoyl]-2-(1-methyl-cyclopentyl)-ethyl]-amide (Example 1q, 100 mg, 0.18 mmol) in CH₂Cl₂ (2 mL) was added Dess-Martin periodinane (100 mg, 0.23 mmol). After stirring for 2 hours, solutions of sodium thiosulfate (10% in water, 0.50 mL) and saturated aqueous sodium bicarbonate (0.50 mL) were added simultaneously to the reaction. The mixture was then extracted with ethyl acetate (2 times). The organic layer was dried with MgSO₄, filtered, and concentrated. Column chromatography (5% methanol:CH₂Cl₂) of the residue provided the tide compound (100 mg, 99%). This compound was purified on a preparative R,R-Whelk-O column by HPLC to yield the two diastereomers of the title compound as solids [first eluting (1A): 30 mg, second eluting (1B): 25 mg]. MS m/z 550.0 (M⁺); The ¹H NMR data of 1A: ¹H NMR (400 Hz, CDCl₃): δ 8.78 (d, 1H), 8.0 (m, 2H), 7.53 (m, 1H), 6.9 (d, 1H), 5.1 (m, 1H), 4.91 (d, 1H), 4.80 (d, 1H), 4.40 (m, 2H), 3.9 (d, 1H), 3.70 (t, 4H), 3.40 (t, 4H), 2.2 (m, 3H), 0.93-1.93 (m, 17H). The ¹H NMR data of 1B: ¹H NMR (400 Hz, CDCl₃): δ 8.7 (d, 1H), 8.0 (m, 2H), 7.53 (m, 1H), 7.2 (d, 1H), 5.1 (m, 1H), 4.8 (d, 1H), 4.48 (d, 1H), 3.86 (d, 1H), 3.75 (m, 4H), 3.40 (m, 4H), 2.2 (m, 2H), 2.05 (m, 1H), 0.93-1.65 (m, 17H).

### Example 2

### Preparation of 2A: Morpholine 4-carboxylic acid {(S)-2-[1-methylcyclohexyl-1-(4S,7R)-7-methyl-3-oxo-1-(1-oxy-pyridine-2-sulfonyl)-azepan-4-ylcarbamoyl]-ethyl}-amide

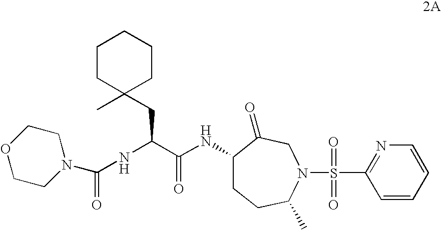

### Preparation of 2B: Morpholine 4-carboxylic acid {(L)-2-[1-methylcyclohexyl-1-(4S,7R)-7-methyl-3-oxo-1-(1-oxy-pyridine-2-sulfonyl)-azepan-4-ylcarbamoyl]-ethyl}-amide

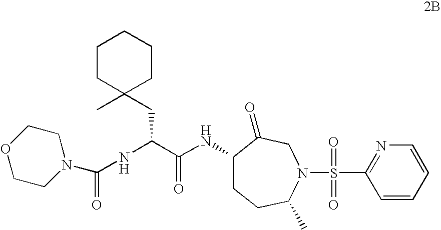

Following the procedure of Example 1(b-r), except substituting “1-methylcyclohexyl” for “1-methylcyclopentyl” gave the title compound: ¹HNMR data of 2A: ¹H NMR (400 Hz, CDCl₃): δ 8.72 (d, 1H), 7.95 (m, 2H), 7.5 (d, 1H), 6.91 (d, 1H), 5.10 (m, 1H), 4.95 (d, 1h), 4.75 (d, 1H), 4.40 (m, 2H), 3.82 (d, 1H), 3.70 (t, 4h), 3.40 (t, 4H), 2.20 (m, 3H), 0.95-1.80 (m, 19H). The ¹H NMR data of 2B: ¹H NMR (400 Hz, CDCl₃): δ 8.70 (d, 1H), 7.95 (m, 2H), 7.52 (m, 1H), 7.2 (d, 1H), 5.10 (m, 1H), 4.83 (d, 1H), 4.70 (d, 1H), 4.44 (m, 2H), 3.82 (d, 1H), 3.70 (t, 4H), 3.40 (t, 4H), 2.2 (m, 2H), 1.9 (m, 1H), 0.95-1.5 (m, 8H).

### Example 3

### Preparation of 3A: Furan-carboxylic acid {(S)-2-[1-methylcyclohexyl-1-(4S,7R)-7-methyl-3-oxo-1-(1-oxy-pyridine-2-sulfonyl)-azepan-4-ylcarbamoyl]-ethyl}-amide

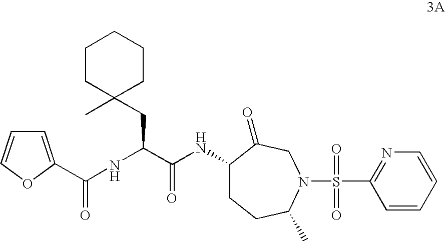

### Preparation 3B: Furan-carboxylic acid {(L)-2-[1-methylcyclohexyl-1-(4S,7R)-7-methyl-3-oxo-1-(1-oxy-pyridine-2-sulfonyl)-azepan-4-ylcarbamoyl]-ethyl}-amide

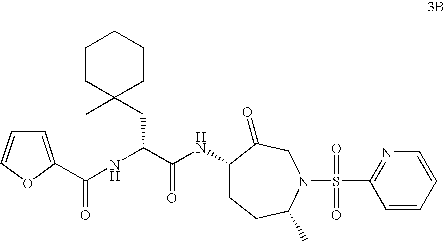

### 3a) Furan-2-carboxylic acid [1-[(3S,4S,7R)-3-hydroxy-7-methyl-1-(pyridine-2 sulfonyl)-azepan-4-ylcarbamoyl]-2-(1-methyl-cyclopentyl)-ethyl]-amide

To a solution of 2-amino-N-[(3S,4S,7R)-3-hydroxy-7-methyl-1-(pyridine-2-sulfonyl)-azepan-4-yl]-3-(1-methyl-cyclohexyl)-propionamide, HCl salt (Example 2o, 357 mg, 0.73 mmol) in DMF, 2-furoic acid (81.8 mg, 0.73 mmol), HBTU (360 mg, 0.95 mmol) and 4-methylmorpholine (369 mg, 3.65 mmol) were added. After the reaction mixture was stirred at room temperature for 16 hours, it was partitioned between ethyl acetate and water. The combined organic phase was washed with water, brine, dried (MgSO₄), filtered and concentrated. Column chromatography (5% methanol:CH₂Cl₂) of the residue provided the N-Boc title compound (376 mg, 94%) MS m/z 547.2 (M⁺).

3b) Following the procedure of Example 1(b-p, r), except substituting “4-methylcyclohexyl” for “1-methylcyclopentyl” and “furan-2-carboxylic acid” for “morpholine 4-carboxylic acid” gave the title compound: The ¹H NMR data of 3A: ¹H NMR (400 Hz, CDCl₃): δ 8.72 (d, 1H), 8.0 (m, 2H), 7.54 (t, 1H), 7.50 (s, 1H), 7.15 (s, 1H), 6.96 (d, 1H), 6.70 (d, 1H), 6.52 (d, 1H), 5.1 (m, 1H), 4.75 (d, 1H), 4.66 (m, 1H), 4.45 (m, 1H), 3.85 (d, 1H), 2.2 (m, 3H), 1.95 (m, 1H), 0.95-1.60 (m, 18H). The ¹H NMR data of 3B: ¹H NMR (400 Hz, CDCl₃): δδ 8.72 (d, 1H), 8.0 (m, 2H), 7.5 (m, 2H), 7.12 (m, 2H), 6.6 (d, 1H), 6.54 (d, 1H), 5.10 (m, 1H), 4.66 (m, 2H), 4.42 (m, 1H), 3.80 (d, 1H), 2.2 (m, 3H), 2.08 (m, 1H), 0.95-1.60 (m, 18H).

### Example 4

### Preparation 4A: Furan-carboxylic acid {(S)-2-[4-methylcyclohexyl-1-(4S,7R)-7-methyl-3-oxo-1-(1-oxy-pyridine-2-sulfonyl)-azepan-4-ylcarbamoyl]-ethyl}-amide

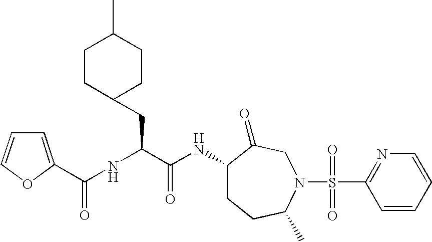

### Preparation 4B: Furan-carboxylic acid {(L)-2-[4-methylcyclohexyl-1-(4S,7R)-7-methyl-3-oxo-1-(1-oxy-pyridine-2-sulfonyl)-azepan-4-ylcarbamoyl]-ethyl}-amide

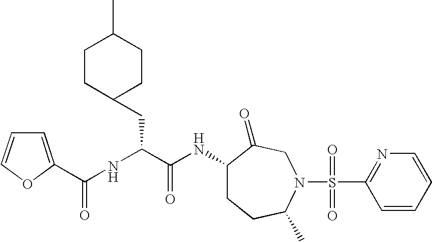

Following the procedure of Example 3(f-r), except substituting “4-methylcyclohexyl” for “1-methylcyclohexyl” gave the title compound: The ¹H NMR data of 4A: ¹H NMR (400 Hz, CDCl₃): δ 8.75 (d, 1H), 8.0 (m, 2H), 7.60 (m, 2H), 7.1 (d, 1H), 6.90 (d, 1H), 6.75 (d, 1H), 6.5 (s, 1H), 5.15 (m, 1H), 4.80 (d, 1H), 4.70 (m, 1H), 4.45 (m, 1H), 3.9 (d, 1H), 2.2 (m, 3H), 0.85-1.90 (m, 19H). The ¹H NMR data of 4B: ¹H NMR (400 Hz, CDCl₃): δ 8.75 (d, 1H), 8.0 (m, 2H), 7.50 (m, 2H), 7.20 (d, 1H), 7.06 (d, 1H), 6.70 (m, 1H), 6.5 (s, 1H), 5.1 (m, 1H), 4.70 (m, 2H), 4.45 (m, 1H), 3.85 (d, 1H), 2.2 (m, 3H), 0.85-1.90 (m, 19H).

### Example 5

### Preparation of 5A: Furan-carboxylic acid {(S)-2-[homocyclopentyl-1-(4S,7R)-7-methyl-3-oxo-1-(1-oxy-pyridine-2-sulfonyl)-azepan-4-ylcarbamoyl]-ethyl}-amide

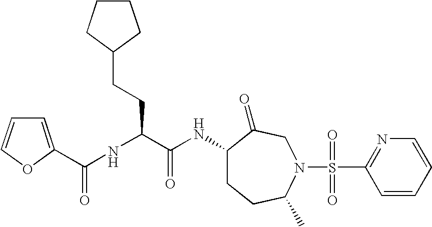

### Preparation of 5B: furan-carboxylic acid {(L)-2-[homocyclopentyl-1-(4S,7R)-7-methyl-3-oxo-1-(1-oxy-pyridine-2-sulfonyl)-azepan-4-ylcarbamoyl]-ethyl}-amide

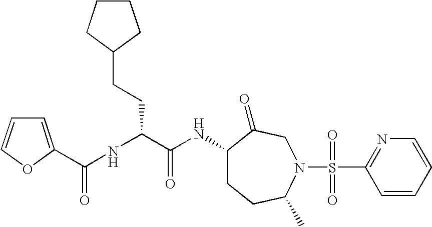

Following the procedure of Example 3(f-r), except substituting “homocyclopentyl” for “1-methylcyclohexyl” gave the title compound: The ¹H NMR data of 5A: ¹H NMR (400 Hz, CDCl₃): δ 8.75 (d, 1H), 8.0 (d, 1H), 7.95 (t, 1H), 7.55 (m, 1H), 7.48 (s, 1H), 7.15 (d, 1H), 6.85 (t, 2H), 6.54 (d, 1H), 5.15 (d, 1H), 4.80 (d, 1H), 4.60 (m, 1H), 4.45 (m, 1H), 3.85 (d, 1H), 2.20 (m, 2H), 1.90 (m, 1H), 1.0-1.83 (m, 17H). The ¹H NMR data of 5B: ¹H NMR (400 Hz, CDCl₃): δ 8.70 (d, 1H), 8.0 (d, 1H), 7.95 (t, 1H), 7.5 (m, 2H), 7.2 (d, 1H), 7.0 (d, 1H), 6.8 (d, 1H), 6.5 (d, 1H), 5.15 (m, 1H), 4.75 (d, 1H), 4.6 (q, 1H), 4.45 (q, 1H), 3.85 (d, 1H), 2.2 (m, 2H), 2.0 (m, 1H), 1.0-1.80 (m, 17H).

### Example 6

### Preparation of 6A: Morpholine 4-carboxylic acid {(S)-2-[homocyclopentyl-1-(4S,7R)-7-methyl-3-oxo-1-(1-oxy-pyridine-2-sulfonyl)-azepan-4-ylcarbamoyl]-ethyl}-amide

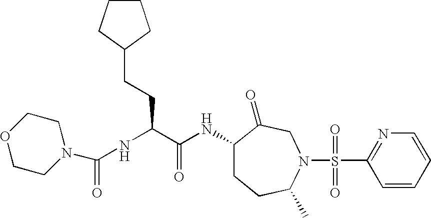

### Preparation of 6B: morpholine 4-carboxylic acid {(L)-2-[homocyclopentyl-1-(4S,7R)-7-methyl-3-oxo-1-(1-oxy-pyridine-2-sulfonyl)-azepan-4-ylcarbamoyl]-ethyl}-amide

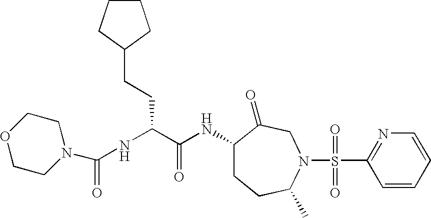

Following the procedure of Example 1(f-r), except substituting “homocyclopentyl” for “1-methylcyclopentyl” gave the title compound: The ¹H NMR data of 6A: ¹H NMR (400 Hz, CDCl₃): δ 8.7 (d, 1H), 7.9 (m, 2H), 7.50 (m, 1H), 7.0 (m, 1H), 5.30 (d, 1H), 5.10 (m, 1H), 4.70 (d, 1H), 4.35 (m, 2H), 3.80 (d, 1H), 3.65 (t, 4H), 3.35 (t, 4H), 2.20 (m, 3H), 0.90-1.75 (m, 17H). The ¹H NMR data of 6B: ¹H NMR (400 Hz, CDCl₃): δ 8.70 (d, 1H), 8.0 (d, 1H), 7.95 (t, 1H), 7.50 (m, 1H), 7.0 (d, 1H), 5.10 (m, 1H), 5.0 (d, 1H), 4.70 (d, 1H), 4.50 (m, 1H), 4.40 (m, 1H), 3.85 (d, 1H), 3.70 (t, 4H), 3.40 (t, 4H), 2.20 (m, 3H), 1.0-1.9 (m, 17H).

### Example 7

### Preparation of 7A: furan-carboxylic acid {(S)-2-[cycloheptyl-1-(4S,7R)-7-methyl-3-oxo-1-(1-oxy-pyridine-2-sulfonyl)-azepan-4-ylcarbamoyl]-ethyl}-amide

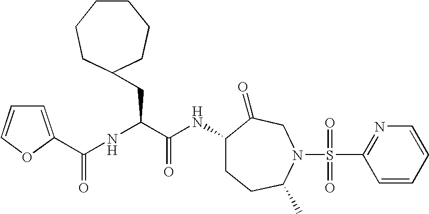

### Preparation of 7B: furan-carboxylic acid {(L)-2-[cycloheptyl-1-(4S,7R)-7-methyl-3-oxo-1-(1-oxy-pyridine-2-sulfonyl)-azepan-4-ylcarbamoyl]-ethyl}-amide

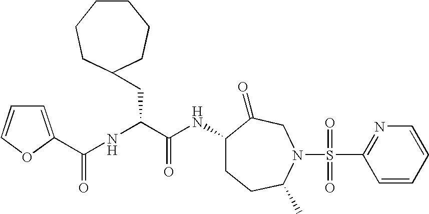

Following the procedure of Example 3(f-r), except substituting “cycloheptyl” for “1-methylcyclohexyl” gave the title compound: The ¹H NMR data of 7A: ¹H NMR (400 Hz, CDCl₃): δ 8.70 (d, 1H), 8.0 (d, 1H), 7.90 (t, 1H), 7.55 (m, 1H), 7.5 (s, 1H), 7.15 (d, 1H), 6.90 (d, 1H), 6.80 (d, 1H), 6.5 (d, 1H), 5.15 (m, 1H), 4.80 (d, 1H), 4.60 (q, 1H), 4.40 (q, 1H), 3.9 (d, 1H), 2.2 (m, 2H), 1.0-1.80 (m, 20H). The ¹H NMR data of 7B: ¹H NMR (400 Hz, CDCl₃): δ 8.70 (d, 1H), 8.0 (d, 1H), 7.90 (t, 1H), 7.55 (d, 1H), 7.50 (s, 1H), 7.15 (d, 1H), 7.05 (d, 1H), 6.7 (d, 1H), 6.5 (d, 1H), 5.10 (m, 1H), 4.75 (d, 1H), 4.65 (m, 1H), 4.5 (m, 1H), 3.85 (d, 1H), 2.20 (m, 2H), 1.0-1.90 (m, 20H).

### Example 8

### Preparation of 8A: morpholine 4-carboxylic acid {(S)-2-[cycloheptyl-1-(4S,7R)-7-methyl-3-oxo-1-(1-oxy-pyridine-2-sulfonyl)-azepan-4-ylcarbamoyl]-ethyl}-amide

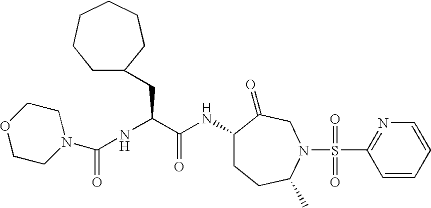

### Preparation of 8B: morpholine 4-carboxylic acid {(L)-2-[cycloheptyl-1-(4S,7R)-7-methyl-3-oxo-1-(1-oxy-pyridine-2-sulfonyl)-azepan-4-ylcarbamoyl]-ethyl}-amide

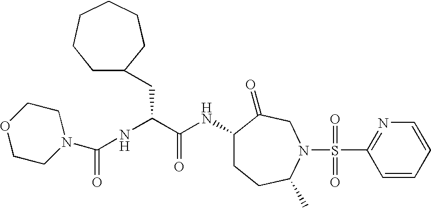

Following the procedure of Example 1(f-r), except substituting “cycloheptyl” for “1-methylcyclopentyl” gave the title compound: The ¹H NMR data of 8A: ¹H NMR (400 Hz, CDCl₃): δ 8.75 (d, 1H), 8.0 (m, 2H), 7.55 (m, 1H), 6.85 (m, 1H), 5.15 (m, 1H), 4.95 (m, 1H), 4.80 (d, 1H), 4.45 (m, 2H), 3.90 (d, 1H), 3.7 (t, 4H), 3.40 (t, 4H), 2.2 (m, 3H), 1.0-1.80 (m, 19H). The ¹H NMR data of 8B: ¹H NMR (400 Hz, CDCl₃): δ 8.75 (d, 1H), 8.0 (m, 2H), 7.55 (m, 1H), 7.10 (m, 1H), 5.10 (m, 1H), 4.80 (m, 1H), 4.75 (d, 1H), 4.40 (m, 2H), 3.85 (d, 1H), 3.70 (t, 4H), 3.40 (t, 4H), 2.2 (m, 3H), 1.0-1.80 (m, 19H).

### Example 9

### Preparation of 9A: furan-carboxylic acid {(S)-2-[cyclopentenyl-1-(4S,7R)-7-methyl-3-oxo-1-(1-oxy-pyridine-2-sulfonyl)-azepan-4-ylcarbamoyl]-ethyl}-amide

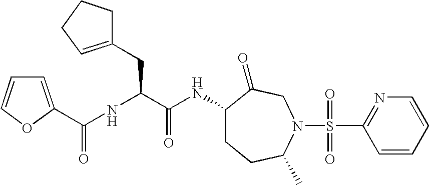

### Preparation of 9B: furan-carboxylic acid {(L)-2-[cyclopentenyl-1-(4S,7R)-7-methyl-3-oxo-1-(1-oxy-pyridine-2-sulfonyl)-azepan-4-ylcarbamoyl]-ethyl}-amide

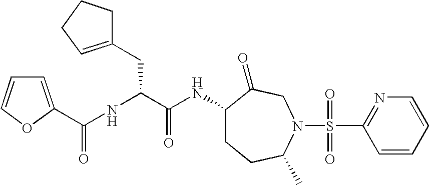

Following the procedure of Example 3(f-r), except substituting “cyclopentenyl” for “1-methylcyclohexyl” gave the title compound: The ¹H NMR data of 9A: ¹H NMR (400 Hz, CDCl₃): δ 8.7 (d, 1H), 8.0 (m, 2H), 7.5 (m, 2H), 7.1 (d, 1H), 7.0 (d, 1H), 6.85 (d, 1H), 6.5 (d, 1H), 5.6 (m, 1H), 5.1 (m, 1H), 4.7 (m, 2H), 4.4 (m, 1H), 3.80 (m, 1H), 2.7 (m, 2H), 2.3 (m, 4H), 2.2 (m, 2H), 1.9 (m, 2H), 1.0-1.7 (m, 5H). The ¹H NMR data of 9B: ¹H NMR (400 Hz, CDCl₃): δ 8.7 (d, 1H), 8.0 (m, 2H), 7.5 (m, 2H), 7.2 (d, 1H), 7.1 (d, 1H), 6.80 (d, 1H), 6.5 (d, 1H), 5.5 (d, 1H), 5.1 (m, 1H), 4.7 (m, 2H), 4.4 (m, 1H), 3.8 (d, 1H), 2.7 (m, 2H), 2.3 (m, 4H), 2.2 (m, 2H), 1.9 (m, 2H), 1.0-1.6 (m, 5H).

### Example 10

### Preparation of 10A: furan-carboxylic acid {(S)-2-[tryptophanyl-1-(4S,7R)-7-methyl-3-oxo-1-(1-oxy-pyridine-2-sulfonyl)-azepan-4-ylcarbamoyl]-ethyl}-amide

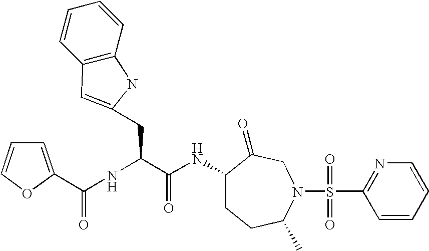

Following the procedure of Example 3(f-r), except substituting “tryptophanyl” for “1-methylcyclhexyl” gave the title compound: ¹H NMR (400 Hz, CDCl₃): δ 8.65 (m, 2H), 8.05 (d, 1H), 7.9 (t, 1H), 7.8 (d, 1H), 7.5 (m, 1H), 7.45 (s, 1H), 7.40 (d, 1H), 7.35 (d, 1H), 7.2 (m, 4H), 6.5 (d, 1H), 5.7 (d, 1H), 5.0 (m, 1H), 3.85 (m, 2H), 3.65 (m, 1H), 3.45 (m, 1H), 3.2 (m, 1H), 3.05 (m, 1H), 2.40 (d, 1H), 0.8-1.6 (m, 6H).

### Example 11

### Preparation of 11A: morpholine 4-carboxylic acid {(S)-2-(7,7-dimethyl-bicyclo[2.2.1]hepty-1-yl)-1-[(4S,7R)-7-methyl-3-oxo-1-(1-oxy-pyridine-2-sulfonyl)-azepan-4-ylcarbamoyl]-ethyl}-amide

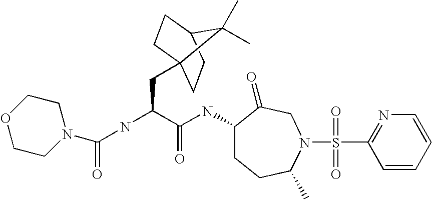

### Preparation of 11B: morpholine 4-carboxylic acid {(L)-2-(7,7-dimethyl-bicyclo[2.2.1]hepty-1-yl)-1-[(4S,7R)-7-methyl-3-oxo-1-(1-oxy-pyridine-2-sulfonyl)-azepan-4-ylcarbamoyl]-ethyl}-amide

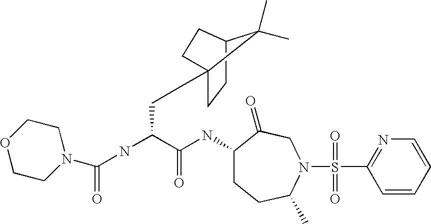

Following the procedure of Example 1(f-r), except substituting “2-(7,7-dimethyl-bicyclo[2.2.1]hepty-1-yl)” for “1-methylcyclopentyl” gave the title compound: The ¹H NMR data of 11A: ¹H NMR (400 Hz, CDCl₃): δ 8.65 (d, 1H), 7.9 (m, 2H), 7.40 (m, 1H), 6.9 (d, 1H), 4.9 (m, 2H), 4.65 (d, 1H), 4.3 (m, 2H), 3.75 (d, 1H), 3.6 (t, 4H), 3.3 (t, 4H), 2.1 (m, 2H), 0.8-1.7 (m, 22H).: The ¹H NMR data of 11B: ¹H NMR (400 Hz, CDCl₃): δ 8.70 (d, 1H), 8.0 (m, 2H), 7.55 (d, 1H), 7.2 (d, 1H), 5.1 (m, 1H), 4.7 (m, 2H), 4.4 (m, 2H), 3.85 (d, 1H), 3.7 (t, 4H), 3.4 (t, 4H), 2.2 (m, 2H), 0.9-1.9 (m, 22H).

### Example 12

### Preparation of 12c: 2-methyl-2H-pyrazole-3-carboxylic acid {(S)-2-cyclopentyl-1-[(4S, 7R)-7-methyl-3-oxo-1-(pyridine-2-sulfonyl)-azepan-4-ylcarbamoyl]-ethyl}-amide

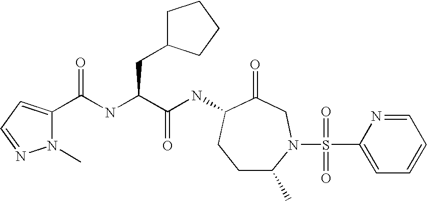

### 12a.) (S)-2-tert-Butoxycarbonylamino-3-cyclopentyl-propionic acid

The solution of (S)-2-amino-3-cyclopentyl-propionic acid (3.0 g, 19.1 mmol) in 30 mL of 1,4dioxane and water (1:1 ratio) was cooled to 0° C., sodium hydroxide (1.5 g, 38 mmol) and di-tert-butyldicarbonate (5.0 g, 22.9 mmol) were added. After stirring at room temperature overnight, the mixture was adjusted to pH 1 with concentrated HCl. The resulting mixture was extracted with ethyl acetate (3×), the combined organic phase was washed with brine, dried over MgSO₄, filtered, and concentrated to give the title compound (4.9 g ). LC-MS m/z 258.2 (M⁺), 1.84 min.

### 12b.) (S)-2-amino-3-cyclopentyl-N-[(3S,4S,7R)-3-hydroxy-7-methyl-1-(pyridine-2-sulfonyl)-azepan-4-yl]-propionamide HCl salt

Following the procedure of Example 1p, (S)-2-tert-butoxycarbonylamino-3-cyclopentyl-propionic acid (4.42 g, 17.2 mmol) and (3S,4S,7R)-4-amino-7-methyl-1-(pyridine-2-sulfonyl)-azepan-3-ol, HCl salt (Example 1o, 7.26 g, 22.5 mmol) were reacted, followed by deprotection with 4N HCl in 1,4-dioxane to give title product (7.9 g, 72%). LC-MS m/z 452.0 (M⁺), 1.54 min.

### 12c.) 2-Methyl-2H-pyrazole-3-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

To a solution of (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 125 mg, 0.27 mmol) in DMF, 2-methyl-2H-pyrazole-3-carboxylic acid (33 mg, 0.26 mmol), HBTU (128 mg, 0.34 mmol) and 4-methylmorpholine (143 μl, 1.3 mmol) were added. After the reaction mixture was stirred at room temperature for 16 hours, it was partitioned between ethyl acetate and water. The combined organic phase was washed with water, brine, dried over MgSO₄, filtered and concentrated. Column chromatography (5% methanol:CH₂Cl₂) of the residue provided 2-methyl-2H-pyrazole-3-carboxylic acid {(S)-2-cyclopentyl-1-[(3S,4S,7R)-7-methyl-1-(pyridine-2-sulfonyl)-azepan-4-ylcarbamoyl]-ethyl}-amide (74 mg, 51%). MS (m/z) 533.0 (M⁺), 1.88 min.

To a stirring solution 2-methyl-2H-pyrazole-3-carboxylic acid {(S)-2-cyclopentyl-1-[(3S,4S,7R)-7-methyl-1-(pyridine-2-sulfonyl)-azepan-4-ylcarbamoyl)-ethyl}-amide (74 mg, 0.14 mmol) in CH₂Cl₂ (2 mL) was added Dess-Martin periodinane (77 mg, 0.18 mmol). After stirring for 3 hours, the mixture was concentrated. The residue was diluted with ethyl acetate and washed with water (2×). The organic layer was dried with MgSO₄, filtered, and concentrated. Column chromatography (5% methanol:CH₂Cl₂) of the residue, followed by recrystallization from dichloromethane and hexane provided the title compound (50 mg, 67%). LC-MS m/z 530.6 (M⁺), 1.85 min.

### Example 13

### Preparation of 13: 1H-pyrazole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

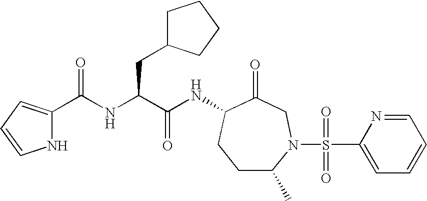

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 200 mg, 0.43 mmol) was coupled with 1H-pyrrole-2-carboxylic acid (53 mg, 0.49 mmol), followed by oxidation with Dess-Martin periodinane (135 mg, 0.32 mmol) to give the title compound (50 mg, 23%). LC-MS m/z 516.2 (M⁺), 1.94 min.

### Example 14

### Preparation of 14: 1-Methyl-1H-pyrrole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

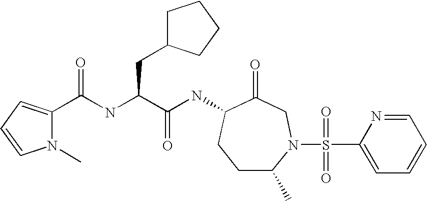

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled with 1-methyl-1H-pyrrole-2-carboxylic acid (50 mg, 0.40 mmol), followed by oxidation with Dess-Martin periodinane (182 mg, 0.43 mmol) to give the title compound (20 mg, 18%). LC-MS m/z 530.0(M⁺), 2.08 min.

### Example 15

### Preparation of 15: Isoxazole-5-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

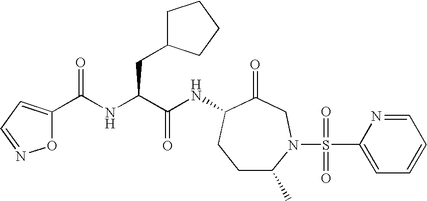

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 100 mg, 0.22 mmol) was coupled with isoxazole-5-carboxylic acid (25 mg, 0.22 mmol), followed by oxidation with Dess-Martin periodinane (15 mg, 0.27 mmol) to give the title compound (20 mg, 18%). LC-MS m/z 518 (M⁺), 1.88 min.

### Example 16

### Preparation of 16b: Thiazole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

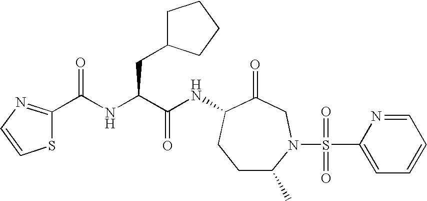

### 16a.) Thiazole-2-carboxylic acid

The mixture of thiazole-2-carboxylic acid ethyl ester (100 mg, 0.64 mmol) and lithium hydroxide monohydrate (134 mg, 3.18 mmol) in 5 mL of methanol was stirred at room temperature for 16 hours. After removing solvent, the residue was acidified with aq. 1N HCl, extracted with ethyl acetate (2×), washed with brine. The combined organic layer was dried over MgSO₄, filtered and concentrated under reduced pressure to give crude material which was used directly in the next step without further purification (53 mg).

### 16b.) Thiazole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 94 mg, 0.20 mmol) was coupled with thiazole-2-carboxylic acid (26 mg, 0.20 mmol), followed by oxidation with Dess-Martin reagent (121 mg, 0.29 mmol) to give the title compound (30 mg, 28%). LC-MS m/z 534.2(M⁺), 2.04 min.

### Example 17

### Preparation of 17: 5-trifluoromethyl-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

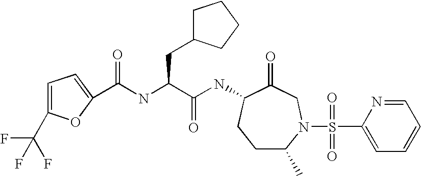

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled with 5-trifluoromethyl-furan-2-carboxylic acid (72 mg, 0.40 mmol), followed by oxidation with Dess-Martin periodinane (182 mg, 0.43 mmol) to give the title compound (29 mg, 15%). LC-MS m/z 585.0(M⁺), 2.25 min.

### Example 18

### Preparation of 18b: 1H-pyrazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

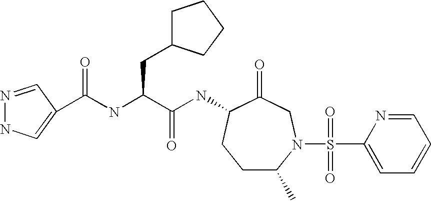

### 18a.) Pyrazole-1,4-dicarboxylic acid-1-tert-butyl ester

Following the general procedure in Example 12a, 1H-pyrazole-4-carboxylic acid (300 mg, 2.68 mmol), sodium hydroxide (214 mg, 5.36 mmol) and di-tert-butyldicarbonate (700 mg, 2.68 mmol) were reacted to give the title compound (153 mg, 27%). ¹H NMR (400 Hz, CDCl3) δ 8.66(s, 1H), 8.14 (s, 1H), 1.70 (s, 9H).

### 18b.) 1H-Pyrazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 166 mg, 0.36 mmol) was coupled with pyrazole-1,4-dicarboxylic acid-1-tert-butyl ester (77 mg, 0.36 mmol), followed by oxidation with Dess-Martin periodinane (182 mg, 0.43 mmol) and then removing the tert-butoxycarbonyl protecting group with 4N HCl to give the title compound (49 mg, 26%). LC-MS m/z 517.2 (M⁺), 1.70 min.

### Example 19

### Preparation of 19: tetrahydrofuran-3-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

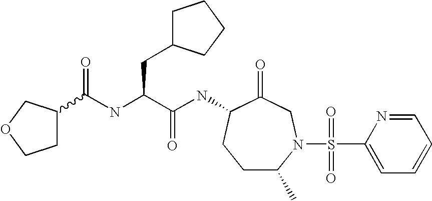

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 200 mg, 0.43 mmol) was coupled with tetrahydro-furan-3-carboxylic acid (75 mg, 0.65 mmol), followed by oxidation with Dess-Martin periodinane (244 mg, 0.57 mmol) to give the title compound (88 mg, 39%). LC-MS m/z 521.2(M⁺), 1.79 min.

### Example 20

### Preparation 20: 4,7-dimethyl-pyrazolo[5,1-c][1,2,4]triazine-3-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

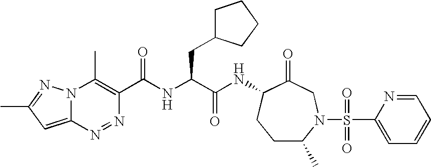

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled with 4,7dimethyl-pyrazolo[5,1-c][1,2,4]triazine-3-carboxylic acid (77 mg, 0.40 mmol), followed by oxidation with Dess-Martin periodinane (182 mg, 0.43 mmol) to give the title compound (3 mg, 2%). LC-MS m/z 597.0 (M⁺), 2.13 min.

### Example 21

### Preparation of 21: 2,7-dimethyl-pyrazolo[5,1-a]pyrimidine-6-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

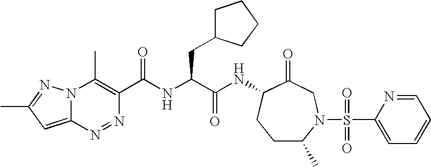

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled with 2,7-dimethyl-pyrazolo[5,1-a]pyrimidine-6-carboxylic acid (70 mg, 0.36 mmol), followed by oxidation with Dess-Martin periodinane (210 mg, 0.49 mmol) to give the title compound (80 mg, 41%). LC-MS m/z 596.0 (M⁺), 1.92 min.

### Example 22

### Preparation of 22: 3-Phenyl-3H-[1,2,3]triazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

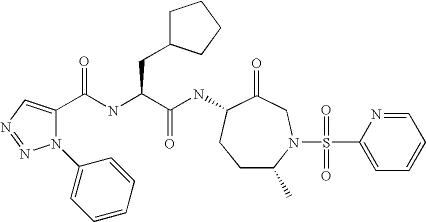

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 180 mg, 0.39 mmol) was coupled with 3-phenyl-3H-[1,2,3]triazole-4-carboxylic acid (81 mg, 0.43 mmol), followed by oxidation with Dess-Martin periodinane (225 mg, 0.53 mmol) to give the title compound (77 mg, 37%). LC-MS m/z 594.2 (M⁺), 2.02 min.

### Example 23

### Preparation of 23: 2-(2,3-Dihydro-benzo[1,4]dioxin-2-yl)-thiazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

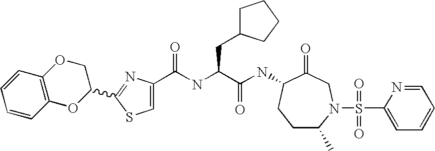

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled with 2-(2,3-dihydro-benzo[1,4]dioxin-2-yl)-thiazole-4-carboxylic acid (99 mg, 0.37 mmol), followed by oxidation with Dess-Martin periodinane (230 mg, 0.54 mmol) to give the title compound (96 mg, 41%). LC-MS m/z 668.0 (M⁺), 2.42 min.

### Example 24

### Preparation of 24: N-{(S)-2-cyclopentyl-1-[(4S,7R)-7-methyl-3-oxo-1-(pyridine-2-sulfonyl)-azepan-4-ylcarbamoyl]-ethyl}-2-pyrazol-1-yl-benzamide

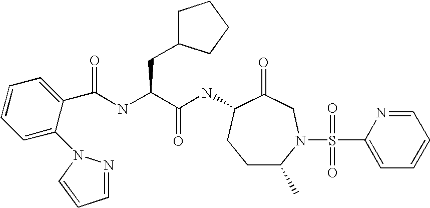

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled with 2-pyrazol-1-yl-benzoic acid (75 mg, 0.40 mmol), followed by oxidation with Dess-Martin periodinane (182 mg, 0.43 mmol) to give the title compound (30 mg, 15%). LC-MS m/z 593.0 (M⁺), 2.00 min.

### Example 25

### Preparation of 25: 4-Methyl-2-phenyl-thiazole-5-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

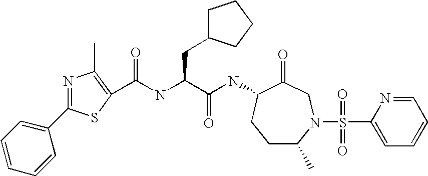

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled 4-methyl-2-phenyl-thiazole-5-carboxylic acid (88 mg, 0.40 mmol), followed by oxidation with Dess-Martin periodinane (182 mg, 0.43 mmol) to give the title compound (117 mg, 57%). LC-MS m/z 624.2 (M⁺), 2.50 min.

### Example 26

### Preparation of 26: 5-(4-Chloro-phenyl)-furan-2-carboxylic acid {(S)-2-cyclopentyl-1-[(4S,7R)-7-methyl-3-oxo-1-pyridine-2-sulfonyl)-azepan-4-ylcarbamoyl]-ethyl}-amide

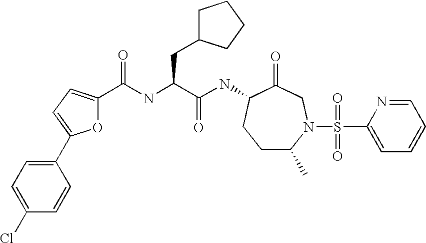

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol) was coupled 5-(4chloro-phenyl)-furan-2-carboxylic acid (82 mg, 0.37 mmol), followed by oxidation with Dess-Martin periodinane (210 mg, 0.50 mmol) to give the title compound (82 mg, 40%). LC-MS m/z 627.2 (M⁺), 2.57 min.

### Example 27

### Preparation of 27: 5-(3-trifluoromethyl-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

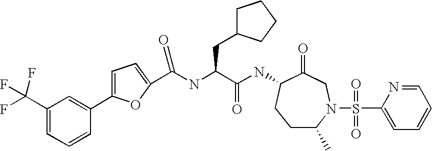

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 100 mg, 0.22 mmol) was coupled 5-(3-trifluoromethyl-phenyl)-furan-2-carboxylic acid (56 mg, 0.22 mmol), followed by oxidation with Dess-Martin periodinane (121 mg, 0.29 mmol) to give the tide compound (60 mg, 40%). LC-MS m/z 661.2 (M⁺), 2.57 min.

### Example 28

### Preparation of 28b: 5-(2-Chloro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

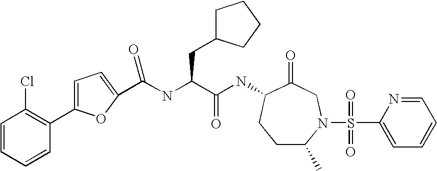

### 28a.) 5-Bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

Following the general procedure described in Example 12c, (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 1.0 g, 2.17 mmol) was coupled 5-bromo-furan-2-carboxylic acid (415 mg, 2.17 mmol) to give the title compound (780 mg, 60%). LC-MS m/z 597.0(M⁺), 1.99 min.

### 28b.) 5-(2-Chloro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

The mixture of 5-bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (25 mg, 0.04 mmol), 2-chlorophenylboronic acid (7 mg, 0.04 mmol), tetrakis-(triphenylphosphine)palladium(o) (2.4 mg, 5%) and potassium carbonate (17.4 mg, 0.13 nmnol) in the mixture of 2 mL of 1,4-dioxane and 0.5 mL of water was heated at 100° C. in the Smith Creator microwave for 800 seconds. The mixture was then diluted with ethyl acetate, washed with water, brine, dried over Anhydrous sodium sulfate, filtered and concentrated to give crude 3-hydroxy intermediate. Upon oxidation as described in Example 12c with Dess-Martin periodinane (50 mg, 0.12 mmol), the title compound was obtained (4 mg, 15%). LC-MS m/z 627.2 (M⁺), 2.49 min.

### Example 29

### Preparation of 29: 5-(4-fluoro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

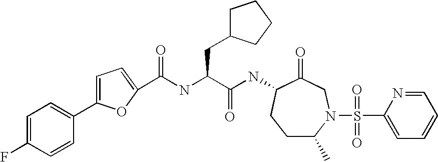

Following the general procedure described in Example 28b, the coupling of 5-bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (Example 28a, 25 mg, 0.04 mmol) with 4-fluorophenylboronic acid (6.4 mg, 0.05 mmol), followed by oxidation with Dess-Martin periodinane (50 mg, 0.12 mmol), the title compound was obtained (6.2 mg, 24%). LC-MS m/z 611.2(M⁺), 2.42min.

### Example 30

### Preparation of 30: 5-(4-methoxy-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

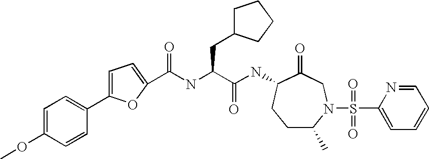

Following the general procedure described in Example 28b, the coupling of 5-bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (Example 28a, 25 mg, 0.04 mmol) with 4-methoxyphenylboronic acid (7.0 mg, 0.05 mmol), followed by oxidation with Dess-Martin periodinane (50 mg, 0.12 mmol), the title compound was obtained (18 mg, 69%). LC-MS m/z 623.4(M⁺), 2.42 min.

### Example 31

### Preparation of 31: 5-phenyl-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

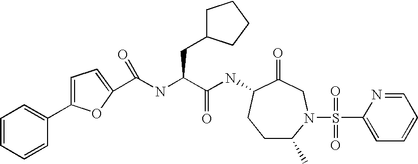

Following the general procedure described in Example 28b, the coupling of 5-bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (Example 28a, 25 mg, 0.04 mmol) with phenylboronic acid (5.6 mg, 0.05 mmol), followed by oxidation with Dess-Martin periodinane (50 mg, 0.12 mmol), the title compound was obtained (10 mg, 42%). LC-MS m/z 593.2(M⁺), 2.40 min.

### Example 32

### Preparation of 32: 5-(4-trifluoromethyl-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

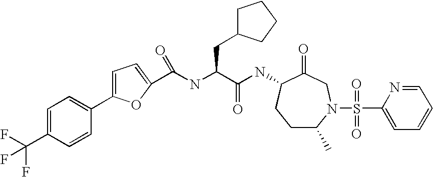

Following the general procedure described in Example 28b, the coupling of 5-bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (Example 28a, 25 mg, 0.04 mmol) with 4-trifluoromethyl-phenylboronic acid (8.7 mg, 0.05 mmol), followed by oxidation with Dess-Martin periodinane (50 mg, 0.12 mmol), the title compound was obtained (15 mg, 56%). LC-MS m/z 661.2(M⁺), 2.59 min.

### Example 33

### Preparation of 33: 5-(3-chloro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

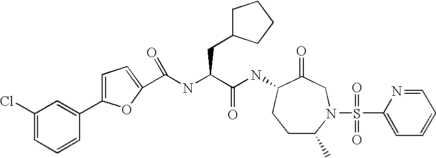

Following the general procedure described in Example 28b, the coupling of 5-bromo-furan-2-carboxylic acid {(S)-2-cyclopentyl-1-[(4S,7R)-7-methyl-3-oxo-1-pyridine-2-sulfonyl)-azepan-4-ylcarbamoyl]-ethyl}-amide (Example 28a, 25 mg, 0.04 mmol) with 3-chloro-phenylboronic acid (7.2 mg, 0.05 mmol), followed by oxidation with Dess-Martin periodinane (23 mg, 0.05 mmol), the title compound was obtained (7 mg, 28%). LC-MS m/z 627.2(M⁺), 2.59 min

### Example 34

### Preparation of 34: 5-(4-methylphenyl)furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

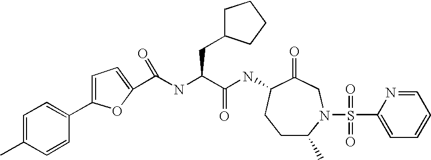

Following the general procedure described in Example 28b, the coupling of 5-bromofuran-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (Example 28a, 25 mg, 0.04 mmol) with p-toylboronic acid (6.3 mg, 0.05 mmol), followed by oxidation with Dess-Martin periodinane (48 mg, 0.11 mmol), the title compound was obtained (6.2 mg, 25%). LC-MS miz 607.4(M⁺), 2.59 min.

### Example 35

### Preparation of 35: 5-(4-acetyl-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

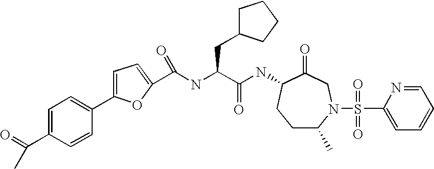

Following the general procedure described in Example 28b, the coupling of 5-bromo-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide (Example 28a, 25 mg, 0.04 mmol) with 4-acetyl-phenylboronic acid (7.5 mg, 0.05 mmol), followed by oxidation with Dess-Martin periodinane (50 mg, 0.12 mmol), the title compound was obtained (15 mg, 59%). LC-MS m/z 635.2(M⁺), 2.30 min

### Example 36

### Preparation of 36: 4-Methyl-piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

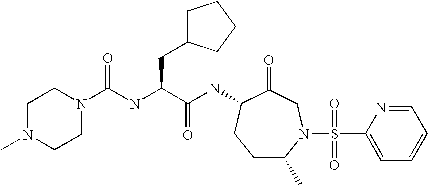

The mixture (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol), 4-methyl-piperazine-1-carbonyl chloride (66 mg, 0.33 mmol) and 0.5 mL of pyridine in 2 mL of dichloromethane was stirred at room temperature for 18 hours. The mixture was then diluted with dichloromethane, washed with water, brine, dried over Anhydrous sodium sulfate, filtered and concentrated to give the crude 3-hydroxy intermediate (117 mg). Upon oxidation as described in Example 12c with Dess-Martin reagent (121 mg, 0.29 mmol), the title compound was obtained (50 mg, 43%). LC-MS m/z 549.2 (M⁺), 1.46 min.

### Example 37

### Preparation of 37c: Piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

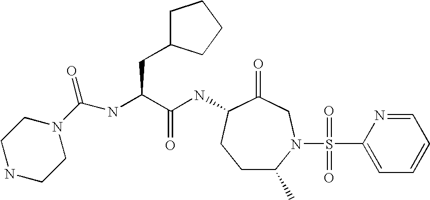

### 37a.) 4-(1-Imidazol-1-yl-methanoyl)-piperazine-1-carboxylic acid tert-butyl ester

The mixture of piperazine-1-carboxylic acid tert-butyl ester (2.0 g, 10.7 mmol) and 1,1-di-imidazol-1-yl-methanone (1.9 g, 11.8 mmol) in 40 mL of tetrohydrofuran was heated to 60° C. for 18 hr. The mixture was concentrated and purified via silica gel column chromatography (ethyl acetate 100%) to provide the title compound (3.9 g, 100%). ¹H NMR (400 Hz, CDCl₃): δ 7.90 (s, 1H), 7.22 (s, 1H), 7.14 (s, 1H), 3.61 (t, 4H), 3.55 (t, 4H), 1.50 (s, 9H).

### 37b.) 4-(1-Imidazol-1-yl-methanoyl)-piperazine-1-carboxylic acid tert-butyl ester methyl iodide salt

The mixture of 4-(1-Imidazol-1-yl-methanoyl)-piperazine-1-carboxylic acid tert-butyl ester (3.9 g, 10.7 mmol) and iodomethane (2.67 mL, 42.8 mmol) in 20 mL of acetonitrile was stirred at room temperature for 18 hr. The mixture was concentrated and the residue was triturated with diethyl ether and hexanes to give the crude material which was used directly in the next step without further purification.

### 37c.) Piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

The mixture of (S)-2-amino-3-cyclopentyl-N-[(3S,4S,7R)-3-hydroxy-7-methyl-1-(pyridine-2-sulfonyl)-azepan-4-yl]-propionamide HCl salt (Example 12b, 150 mg, 0.33 mmol), 4-(1-imidazol-1-yl-methanoyl)-piperazine-1-carboxylic acid tert-butyl ester methyl iodide salt (139 mg, 0.33 mmol) and triethylamine was heated at 70° C. for 10 min in the Smithcreator microwave to give the 3-hydroxy intermediate. Upon oxidation with Dess-Martin periodinane (182 mg, 0.43 mmol) followed by removal of the tert-butoxycarbonyl protecting group with 4N HCl the title compound was obtained (50 mg, 28%). LC-MS m/z 535.2 (M⁺), 1.44 min.

### Example 38

### Preparation of 38h: 5-Trifluoromethyl-furan-2-carboxylic acid {(S)-2-cyclohexyl-1-[(s)-3-oxo-1-(pyridine-2-sulfonyl)-azepan-4-ylcarbamoyl]-ethyl}-amide

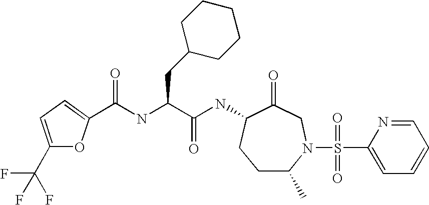

### 38a.) (S)—(R)-1-Oxiranyl-prop-2-en-1-ol

To a mixture of 4A molecular sieves (20 g) in 500 mL of dichloromethane at −30° C., was added titanium (IV) isopropoxide (17.7 mL, 59.4 mmol), followed by diisopropyl D-tartrate (16.4 mL, 77.3 mmol). The mixture was stirred at −30° C. for 30 min. 1,4-pentadien-3-ol (50 g, 0.59 mol) was added, follewed by cumene hydroperoxide (153 mL, 0.92 mol). After standing at −15° C. for 72 hr, 300 mL of diethyl ether and 50 mL of saturated aqueous Anhydrous sodium sulfate were added. The resulting mixture was stirred for 3.5 hours at room temperature, then filtered through celite. The organic phase was separated and concentrated. Column chromatography (5% diethyl ether/95% hextan to 50% diethyl ether/50% hexane) provided the crude title compound (56 g), which was used in next step without further purification.

### 38b.) 2-((R)—(S)-1-Oxiranyl-allyl)-isoindole-1,3-dione

The mixture of (S)—(R)-1-Oxiranyl-prop-2-en-1-ol (50 g, 0.5 mol), triphenylphosphine (196 g, 0.75 mol) and phthalimide (110 g, 0.75 mmol) in 300 mL of toluene was cooled to 0° C. where diisopropyl azodicarboxylate (147 mL 0.75 mol) in 100 mL of toluene was added dropwise. The resulting mixture was allowed to warm to ambient temperature and stirred for 18 hours. After standing at −15° C. for 60 min, the mixture was filtered and washed with toluene. The filtrate was washed with aqueous 1N NaOH (2×), water, then concentrated. Flash chromatography of the residue (20% diethyl ether/80% hexanes), followed by recrystallization from methanol provided the desired product (41 g, 36% two steps). ¹H NMR (400 Hz, DMSO-d6): δ 7.87-7.93 (m, 4H), 6.05-6.13 (m, 1H), 5.37-5.34 (m, 2H), 4.40-4.43 (m, 1H), 3.59-3.62 (m, 1H), 2.51-2.92 (m, 2H).

### 38c.) Pyridine-2-sulfonic acid allyl-[(2S,3S)-3-(1,3-dioxo-1,3-dihydro-isoindol-2-yl)-2-hydroxy-pent-4-enyl]-amide

To a mixture of 2-((R)—(S)-1-oxiranyl-allyl)-isoindole-1,3-dione (30 g, 132 mmol) and pyridine-2-sulfonic acid allylamide (26 g, 132 mmol) in 300 mL of isopropanol at room temperature, was added 1,8-diazabicyclo[5.4.0]undec-7-ene (1.97 mL, 13.2 mmol). The mixture was heated to reflux for 18 hours. The mixture was then cooled to ambient temperature, diluted with dichloromethane, washed with aqueous 1N HCl, water, and brine. The organic phase was dried over anhydrous sodium sulfate, filtered and concentrated to give the crude material which was used in the next step without further purification.

### 38d.) 2-[(3S,4S)-3-Hydroxy-1-(pyridine-2-sulfonyl)-2,3,4,7-tetrahydro-1H-azepin-4-yl]-isoindole-1,3-dione

The mixture of pyridine-2-sulfonic acid allyl-[(2S,3S)-3-(1,3-dioxo-1,3-dihydro-isoindol-2-yl)-2-hydroxy-pent-4-enyl]-amide (53 g, 12c4 mmol) in 700 mL of 1,2-dichloroethane was degassed for 5 min. Grubbs reagent (5.27 g, 6.21 mmol) was then added. The mixture was heated to 70° C. for 18 hours. The mixture was cooled to room temperature and filtered. The solid was washed with ethyl acetate and dried to yield the title compound (22 g, 44%). ¹H NMR (400 Hz, DMSO-d6): δ 8.78 (s, 1H), 8.13 (t, 1H), 7.96 (d, 1H), 7.85-7.89 (m, 4H), 7.73(m, 1H), 5.67-5.74 (m, 2H), 5.51 (m, 1H), 4.93-4.95 (m, 1H), 4.23-4.25 (m, 1H), 3.95 (m, 2H), 3.5980-3.83 (m, 1H), 3.36-3.39 (m, 1H).

### 38e.) 2-[(3S,4S)-3-Hydroxy-1-(pyridine-2-sulfonyl)-azepan-4-yl]-isoindole-1,3-dione

To the mixture of 2-[(3S,4S)-3-hydroxy-1-(pyridine-2-sulfonyl)-2,3,4,7-tetrahydro-1H-azepin-4-yl]-isoindole-1,3-dione (5 g, 12c.5 mmol) in 80 mL of N,N-dimethylformamide and 20 mL of ethanol was bubbled argon for 5 min, followed by addition of palladium (10 wt % on activated carbon, 2.5 g). The mixture was hydrogenated on a Parr hydrogenation apparatus at 50° C. for 2 hours and at room temperature for 16 hours. The mixture was filtered through celite and the filtrate concentrated to give the desired product (4.6 g, 91%). LC-MS m/z 402.2(M⁺), 1.62 min

### 38f.) (3S,4S)-4-Amino-1-(pyridine-2-sulfonyl)-azepan-3-ol

To a suspension of 2-[(3S,4S)-3-hydroxy-1-(pyridine-2-sulfonyl)-azepan-4-yl]-isoindole-1,3-dione (29 g, 72 mmol) in 500 mL of ethanol, hydrazine (8.8 mL, 281 mmol) was added. The mixture was heated at reflux for 2 hours. After cooling, the mixture was filtered and the filtrate concentrated. The residue was diluted with dichloromethane, washed with aqueous 10% Na₂CO₃, water, and brine. The organic phase was dried over anhydrous sodium sulfate, filtered and concentrated to give desired product (15 g, 76%). LC-MS m/z 272.0(M⁺), 0.75 min,

### 38g.) (S)-2-Amino-3-cyclohexyl-N-[(3S,4S)-3-hydroxy-1-(pyridine-2-sulfonyl)-azepan-4-yl]-propionamide HCl salt

Following the procedure of Example 1p, (S)-2-tert-Butoxycarbonylamino-3-cyclohexyl-propionic acid (5.58 g, 20.6 mmol) and (3S,4S)-4-Amino-1-(pyridine-2-sulfonyl)-azepan-3-ol, (Example 38f, 5.07 g, 18.7 mmol) were reacted, followed by deprotection with 4N HCl in 1,4-dioxane to give the title product. LC-MS m/z 425.2 (M⁺), 1.33 min.

### 38h.) 5-Trifluoromethyl-furan-2-carboxylic acid {(S)-2-cyclohexyl-1-[(s)-3-oxo-1-(pyridine-2-sulfonyl)-azepan-4-ylcarbamoyl]-ethyl}-amide

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (150 mg, 0.33 mmol) was coupled with 5-trifluoromethyl-furan-2-carboxylic acid (72 mg, 0.40 mmol), followed by oxidation with Dess-Martin periodinane (182 mg, 0.43 mmol) to give the title compound (17 mg, 6%). LC-MS m/z 585.2 (M⁺), 2.34 min.

### Example 39

### Preparation of 39: 2,4-Dimethyl-thiazole-5-carboxylic acid {(S)-2-cyclohexyl-1-[(s)-3-oxo-1-(pyridine-2-sulfonyl)-azepan-4-ylcarbamoyl]-ethyl}-amide

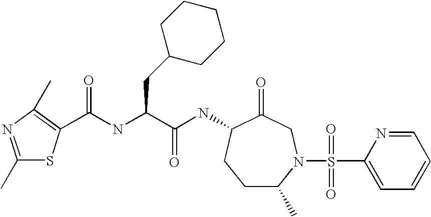

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 2,4-dimethyl-thiazole-5-carboxylic acid (63 mg, 0.40 mmol), followed by oxidation with Dess-Martin periodinane (182 mg, 0.43 mmol) to give the title compound (88 mg, 48%). LC-MS m/z 562.0 (M⁺), 2.09 min.

### Example 40

### Preparation of 40: 5-Methyl-pyrazine-2-carboxylic acid {(S)-2-cyclohexyl-1-[(s)-3-oxo-1-(pyridine-2-sulfonyl)-azepan-4-ylcarbamoyl]-ethyl}-amide

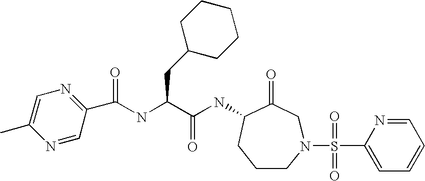

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 5-methyl-pyrazine-2-carboxylic acid (55 mg, 0.40 mmol), followed by oxidation with Dess-Martin periodinane (182 mg, 0.43 mmol) to give the title compound (62 mg, 35%). LC-MS m/z 543.2 (M⁺), 2.07 min.

### Example 41

### Preparation of 41: 1-Methyl-1H-imidazole-2-carboxylic acid {(S)-2-cyclohexyl-1-[(s)-3-oxo-1-(pyridine-2-sulfonyl)-azepan-4-ylcarbamoyl]-ethyl}-amide

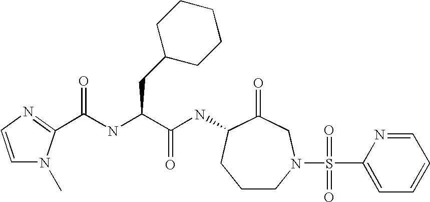

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 1-methyl-1H-imidazole-2-carboxylic acid (50 mg, 0.40 mmol), followed by oxidation with Dess-Martin periodinane (182 mg, 0.43 mmol) to give the title compound (70 mg, 40%). LC-MS m/z 531.0 (M⁺).

### Example 42

### Preparation of 42: 1H-Pyrazol-4-carboxylic acid {(S)-2-cyclohexyl-1-[(s)-3-oxo-1-(pyridine-2-sulfonyl)-azepan-4-ylcarbamoyl]-ethyl}-amide

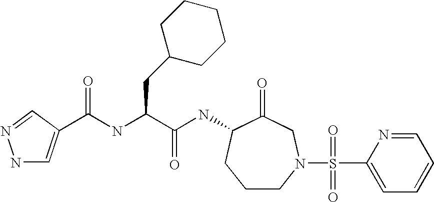

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 165 mg, 0.36 mmol) was coupled with pyrazole-1,4-dicarboxylic acid-1-tert-butyl ester (Example 18a, 77 mg, 0.36 mmol), followed by removal of the tert-butoxycarbonyl protecting group with 4N HCl. Oxidation with Dess-Martin periodinane (182 mg, 0.43 mmol) gave the title compound (40 mg, 21%). LC-MS m/z 517.2 (M⁺), 1.72 min.

### Example 43

### Preparation of 43: 4-Methyl-2-phenyl-thiazole-5-carboxylic acid {(S)-2-cyclohexyl-1-[(s)-3-oxo-1-(pyridine-2-sulfonyl)-azepan-4-ylcarbamoyl]-ethyl}-amide

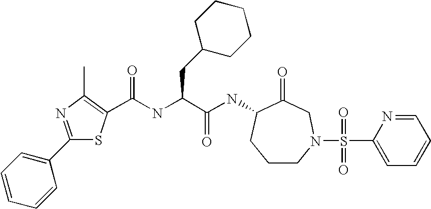

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 4-methyl-2-phenyl-thiazole-5-carboxylic acid (88 mg, 0.40 mmol), followed by oxidation with Dess-Martin periodinane (182 mg, 0.43 mmol) to give the tide compound (117 mg, 57%). LC-MS m/z 624.2 (M⁺), 2.50 min.

### Example 44

### Preparation of 44: 2,5-Dimethyl-furan-3-carboxylic acid {(S)-2-cyclohexyl-1-[(S)-3-oxo-1-(pyridine-2-sulfonyl)-azepan-4-ylcarbamoyl]-ethyl}-amide

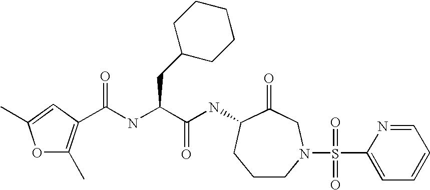

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 2,5-dimethyl-furan-3-carboxylic acid (56 mg, 0.40 mmol), followed by oxidation with Dess-Martin periodinane (182 mg, 0.43 mmol) to give the title compound (42 mg, 23%). LC-MS m/z 545.0 (M⁺), 2.27 min.

### Example 45

### Preparation of 45: 2-Methyl-furan-3-carboxylic acid {(S)-2-cyclohexyl-1-[(S)-3-oxo-1-(pyridine-2-sulfonyl)-azepan-4-ylcarbamoyl]-ethyl}-amide

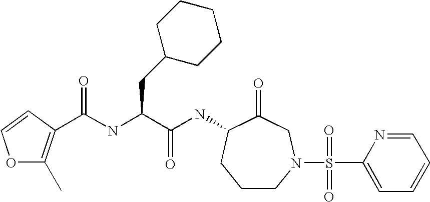

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 2-methyl-furan-3-carboxylic acid (50 mg, 0.40 mmol), followed by oxidation with Dess-Martin periodinane (182 mg, 0.43 mmol) to give the title compound (39 mg, 22%). LC-MS m/z 531.0 (M⁺), 2.13 min.

### Example 46

### Preparation of 46: Isoxazole-5-carboxylic acid {(S)-2-cyclohexyl-1-[(S)-3-oxo-1-(pyridine-2-sulfonyl)-azepan-4-ylcarbamoyl]-ethyl}-amide

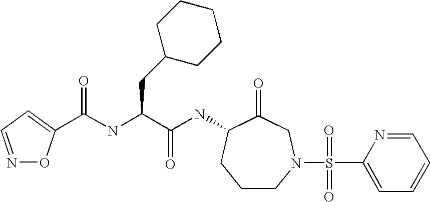

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with isoxazole-5-carboxylic acid (25 mg, 0.22 mmol), followed by oxidation with Dess-Martin periodinane (182 mg, 0.43 mmol) to give the title compound (40 mg, 35%). LC-MS m/z 518.2 (M⁺), 1.94 min.

### Example 47

### Preparation of 47: 5-Methyl-isoxazole-3-carboxylic acid {(S)-2-cyclohexyl-1-[(S)-3-oxo-1-(pyridine-2-sulfonyl)-azepan-4-ylcarbamoyl]-ethyl}-amide

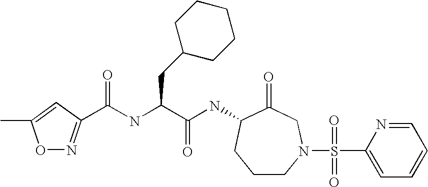

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 5-methyl-isoxazole-3-carboxylic acid (28 mg, 0.22 mmol), followed by oxidation with Dess-Martin periodinane (182 mg, 0.43 mmol) to give the title compound (53 mg, 45%). LC-MS m/z 531.8 (M⁺), 2.14 min.

### Example 48

### Preparation of 48: 5-Methyl-isoxazole-4-carboxylic acid {(S)-2-cyclohexyl-1-[(S)-3-oxo-1-(pyridine-2-sulfonyl)-azepan-4-ylcarbamoyl]ethyl}-amide

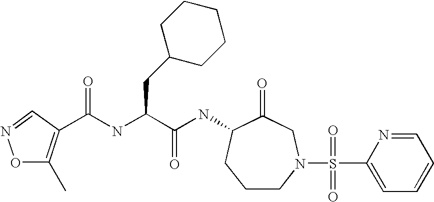

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 5-methyl-isoxazole-carboxylic acid (28 mg, 0.22 mmol), followed by oxidation with Dess-Martin periodinane (121 mg, 0.29 mmol) to give the title compound (26 mg, 22%). LC-MS m/z 532.0 (M⁺), 2.04 min.

### Example 49

### Preparation of 49: 3-Methyl-isoxazole-4-carboxylic acid {(S)-2-cyclohexyl-1-[(S)-3-oxo-1-(pyridine-2-sulfonyl)-azepan-4-ylcarbamoyl]-ethyl}-amide

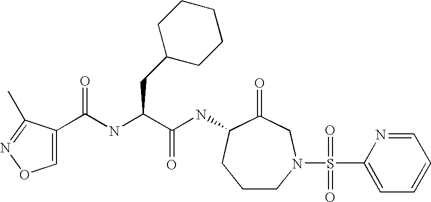

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 3-methyl-isoxazole-4-carboxylic acid (28 mg, 0.22 mmol), followed by oxidation with Dess-Martin periodinane (121 mg, 0.29 mmol) to give the title compound (16 mg, 14%). LC-MS m/z 532.0 (M⁺), 2.04 min.

### Example 50

### Preparation of 50: 2-Methyl-2H-pyrazole-3-carboxylic acid {(S)-2-cyclohexyl-1-[(S)-3-oxo-1-(pyridine-2-sulfonyl)-azepan-4-ylcarbamoyl]-ethyl}-amide

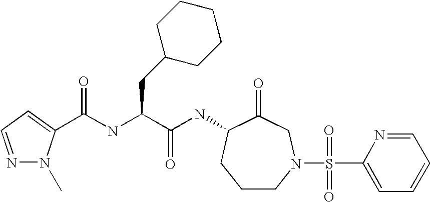

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 2-methyl-2H-pyrazole-3-carboxylic acid (28 mg, 0.22 mmol), followed by oxidation with Dess-Martin periodinane (121 mg, 0.29 mmol) to give the title compound (20 mg, 16%). LC-MS m/z 531.2 (M⁺), 1.97 min.

### Example 51

### Preparation of 51: Pyrazine-2-carboxylic acid {(S)-2-cyclohexyl-1-[(S)-3-oxo-1-(pyridine-2-sulfonyl)-azepan-4-ylcarbamoyl]-ethyl}-amide

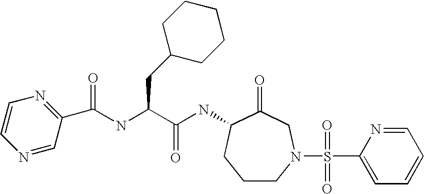

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with pyrazine-2-carboxylic acid (27 mg, 0.22 mmol), followed by oxidation with Dess-Martin periodinane (121 mg, 0.29 mmol) to give the title compound (15 mg, 13%). LC-MS m/z 529.2 (M⁺), 2.02 min.

### Example 52

### Preparation of 52: Thiazole-2-carboxylic acid {(S)-2-cyclohexyl-1-[(S)-3-oxo-1-(pyridine-2-sulfonyl)-azepan-4-ylcarbamoyl]-ethyl}-amide

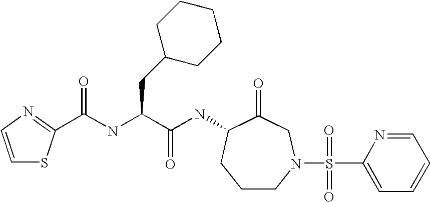

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 94 mg, 0.20 mmol) was coupled with thiazole-2-carboxylic acid (Example 16a, 26 mg, 0.20 mmol), followed by oxidation with Dess-Martin periodinane (121 mg, 0.29 mmol) to give the title compound (45 mg, 28%). LC-MS m/z 534.0 (M⁺), 2.10 min.

### Example 53

### Preparation of 53: 2-Methyl-thiazole-4-carboxylic acid {(S)-2-cyclohexyl-1-[(S)-3-oxo-1-(pyridine-2-sulfonyl)-azepan-4-ylcarbamoyl]-ethyl}-amide

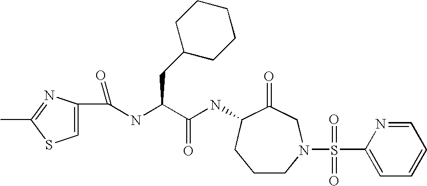

Following the general procedure described in Example 12c, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 2-methyl-thiazole-4-carboxylic acid (32 mg, 0.22 mmol), followed by oxidation with Dess-Martin periodinane (121 mg, 0.29 mmol) to give the title compound (34 mg, 28%). LC-MS m/z 548.0 (M⁺), 2.10 min.

### Example 54

### Preparation of 54: (S)-3-Cyclohexyl-N-[(S)-3-oxo-1-(pyridine-2-sulfonyl)-azepan-4-yl]-2-(thiophene-2-sulfonylamino)-propionamide

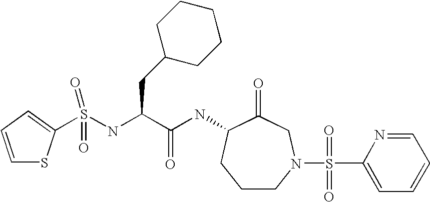

The mixture of (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol), thiophene-2-sulfonyl chloride (40 mg, 0.22 mmol) and triethyl amine (0.15 mL, 1.1 mmol) in 1 mL of dichloromethane was stirred at room temperature for 18 hours. The mixture was then diluted with ethyl acetate, washed with water and brine and dried over anhydrous sodium sulfate, filtered and concentrated to give the crude 3-hydroxy intermediate. Upon oxidation with Dess-Martin periodinane (121 mg, 0.29 mmol) the title compound was obtained (95 mg, 76%). LC-MS m/z 569.0 (M⁺), 2.15 min.

### Example 55

### Preparation of 55: (S)-3-Cyclohexyl-2-(1-methyl-1H-imdiazole-4-sulfonylamino)-N-[(S)-3-oxo-1-(pyridine-2-sulfonyl)-azepan-4-yl]-propionamide

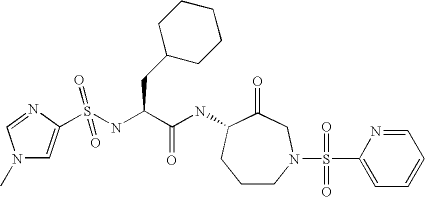

Following the general procedure described in Example 54, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 1-methyl-1H-imdiazole-4-sulfonyl chloride (20 mg, 0.11 mmol), followed by oxidation with Dess-Martin periodinane (121 mg, 0.29 mmol) to give the title compound (19 mg, 30%). LC-MS m/z 567.4 (M⁺), 1.77 min.

### Example 56

### Preparation of 56: (S)-3-Cyclohexyl-2-(3,5-dimethyl-isoxazole-4-sulfonylamino)-N-[(S)-3-oxo-1-(pyridine-2-sulfonyl)-azepan-4-yl]-propionamide

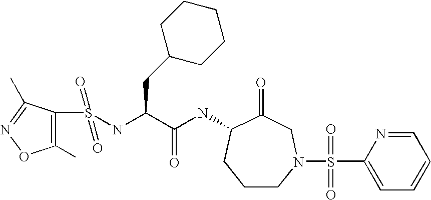

Following the general procedure described in Example 54, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 3,5-dimethyl-isoxazole-4-sulfonyl chloride (43 mg, 0.22 mmol), followed by oxidation with Dess-Martin periodinane (121 mg, 0.29 mmol) to give the title compound (46 mg, 36%). LC-MS m/z 582.4 (M⁺), 2.08 min.

### Example 57

### Preparation of 57b: (S)-3-Cyclohexyl-2-(furan-2-sulfonylamino)-N-[(S)-3-oxo-1-(pyridine-2-sulfonyl)-azepan-4-yl]-propionamide

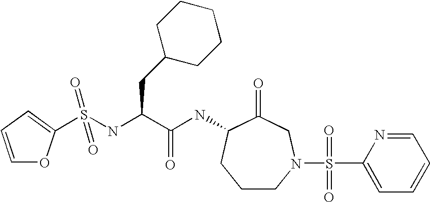

### 57a.) Furan-2-sulfonyl chloride

To a solution of furan (1.0 g, 15 mmol) in 10 mL of tetrahydrofuran at −78° C., n-butyl lithium (1.6M in hexane, 10 mL, 16 mmol) was added dropwise. The mixture was stirred at −78° C. for 30 min, after which time sulfur dioxide was bubbled through the solution for 5 min. The resulting mixture was then stirred for an additional 1 hour and warmed to ambient temperature. After re-cooling to 0° C., sulphuryl chloride (1.17 mL, 14.7 mmol) was added and the mixture stirred for 2 hours. The mixture was diluted with ethyl acetate, washed with water and brine, dried over MgSO₄, filtered and concentrated to give crude material (1.5 g) which was used in the next step without further purification.

### 57b.) (S)-3-Cyclohexyl-2-(furan-2-sulfonylamino)-N-[(S)-3-oxo-1-(pyridine-2-sulfonyl)-azepan-4-yl]-propionamide

Following the general procedure described in Example 54, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with furan-2-sulfonyl chloride (54 mg, 0.33 mmol), followed by oxidation with Dess-Martin periodinane (121 mg, 0.29 mmol) to give the title compound (34 mg, 19%). LC-MS m/z 553.2 (M⁺), 2.00 min.

### Example 58

### Preparation of 58: (S)-3-Cyclohexyl-N-[(S)-3-oxo-1-(pyridine-2-sulfonyl)-azepan-4-yl]-2-(pyridine-2-sulfonylamino)-propionamide

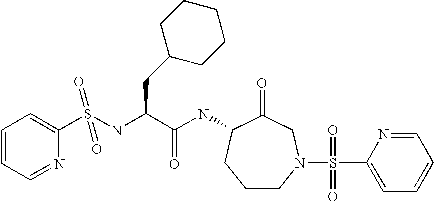

Following the general procedure described in Example 54, (S)-2-amino-3-cyclohexyl-N-[(3S,4S)-3-hydroxy-1-(pyridine-2-sulfonyl)-azepan-4-yl)-propionamide HCl salt (Example 38g, 100 mg, 0.22 mmol) was coupled with pyridine-2-sulfonyl chloride (39 mg, 0.22 mmol), followed by oxidation with Dess-Martin periodinane (121 mg, 0.29 mmol) to give the title compound (19 mg, 15%). LC-MS m/z 564.0 (M⁺), 1.88 min.

### Example 59

### Preparation of 59: (S)-3-Cyclohexyl-2-(morpholine-4-sulfonylamino)-N-[(S)-3-oxo-1-(pyridine-2-sulfonyl)-azepan-4-yl]-propionamide

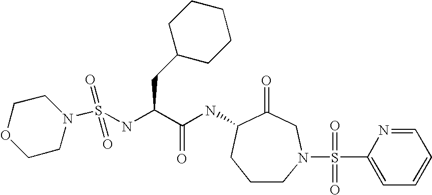

Following the general procedure described in Example 54, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with morpholine-4-sulfonyl chloride (prepared from coupling of morpholing with sulphuryl chloride, 41 mg, 0.22 mmol), followed by oxidation with Dess-Martin periodinane (121 mg, 0.29 mmol) to give the title compound (7 mg, 6%). LC-MS m/z 572.0 (M⁺).

### Example 60

### Preparation of 60: (S)-3-Cyclohexyl-2-(5-isoxazol-3-yl-thiophene-2-sulfonylamino)-N-[(S)-3-oxo-1-(pyridine-2-sulfonyl)-azepan-4-yl]-propionamide

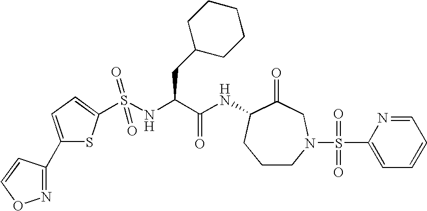

Following the general procedure described in Example 54, (S)-2-amino-3-cyclohexyl-N-[(3S,4S)-3-hydroxy-1-(pyridine-2-sulfonyl)-azepan-4-yl]-propionamide HCl salt (Example 38g, 100 mg, 0.22 mmol) was coupled with 5-isoxazol-3-yl-thiophene-2-sulfonyl chloride (55 mg, 0.22 mmol), followed by oxidation with Dess-Martin periodinane (121 mg, 0.29 mmol) to give the title compound (38 mg, 27%). LC-MS m/z 636.2 (M⁺), 2.12 min.

### Example 61

### Preparation of 61: 4-{(S)-3-Cyclopentyl-2-[(1-furan-2-yl-methanoyl)-amino]-propanoylamino}-3-oxo-azepane-1-carboxylic acid benzyl ester (first diastereomer eluted)

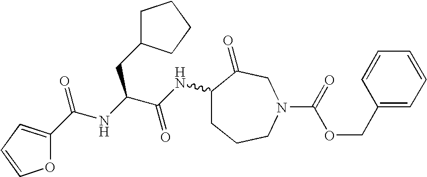

### 61a.) Allyl-pent-4-enyl-carbamic acid benzyl ester

To a suspension of NaH (1.83 g, 76.33 mmol of 90% NaH) in DMF was added benzyl allyl-carbamic acid benzyl ester (7.3 g, 38.2 mmol) in a dropwise fashion. The mixture was stirred at room temperature for approximately 10 minutes whereupon 5-bromo-1-pentene (6.78 mL, 57.24 mmol) was added in a dropwise fashion. The reaction was heated to 40° C. for approximately 4 hours whereupon the reaction was partitioned between dichloromethane and water. The organic layer was washed with water (2×'s), brine, dried (MgSO₄), filtered and concentrated. Column chromatography of the residue (10% ethyl acetate:hexanes) provided 10.3 grams of the title compound as an oil: MS(EI) 260 (M+H⁺).

### 61b.) 2,3,4,7-Tetrahydro-azepine-1-carboxylic acid benzyl ester

To a solution of compound of Example 1a (50 g) in dichloromethane was added bis(tricyclohexylphosphine)benzylidine ruthenium (W) dichloride (5.0 g). The reaction was heated to reflux until complete as determined by TLC analysis. The reaction was concentrated in vacuo. Column chromatography of the residue (50% dichloromethane:hexanes) gave 35 g of the title compound: MS(EI) 232 (M+H⁺).

### 61c.) 8-Oxa-3-aza-bicyclo[5.1.0]octane-3-carboxylic acid benzyl ester

To a solution of the compound of Example 1b (35 g, 1.5 mol) in CH₂Cl₂ was added m-CPBA (78 g, 0.45 mol). The mixture was stirred overnight at room temperature whereupon it was filtered to remove the solids. The filtrate was washed with water and saturated NaHCO₃ (several times). The organic layer was dried (MgSO₄), filtered and concentrated to give 35 g of the title compound which was of sufficient purity to use in the next step: MS(EI) 248 (M+H⁺), 270 (M+Na⁺).

### 61d.) 4-azido-3-hydroxy-azepane-1-carboxylic acid benzyl ester

To a solution of the epoxide from Example 1c (2.0 g, 8.1 mmol) in methanol:water (8:1 solution) was added NH₄Cl (1.29 g, 24.3 mmol) and sodium azide (1.58 g, 24.30 mmol). The reaction was heated to 40° C. until complete consumption of the starting epoxide was observed by TLC analysis. The majority of the solvent was removed in vacuo and the remaining solution was partitioned between ethyl acetate and pH 4 buffer. The organic layer was washed with sat. NaHCO₃, water, brine dried (MgSO₄), filtered and concentrated. Column chromatography (20% ethyl acetate:hexanes) of the residue provided 1.3 g of the title compound: MS(E) 291 (M+H⁺) plus 0.14 g of trans-4-hydroxy-3-azido-hexahydro-1H-azepine

### 61e.) 4-Amino-3-hydroxy-azepane-1-carboxylic acid benzyl ester

To a solution of the azido alcohol of Example 1d (1.1 g, 3.79 mmol) in methanol was added triethylamine (1.5 mL, 11.37 mmol) and 1,3-propanedithiol (1.1 mL, 11.37 mL). The reaction was stirred until complete consumption of the starting material was observed by TLC analysis whereupon the reaction was concentrated in vacuo. Column chromatography of the residue (20% methanol:dichloromethane) provided 0.72 g of the title compound: MS(EI) 265 (M+H⁺).

### 61f.) 4-((S)-2-tert-Butoxycarbonylamino-3-cyclopentyl-propanoylamino)-3-hydroxy-azepan-1-carboxylic acid benzyl ester

To a mixture of the hydrochloride salt of 4-amino-3-hydroxy-azepane-1-carboxylic acid benzyl ester (Example 61e, 1.17 g, 3.89 mmol), (S)-2-tert-butoxycarbonylamino-3-cyclopentylpropionic acid (1.0 g, 3.89 mmol), and 4-methylmorpholine (1.985 g, 19.45 mmol) stirring in DMF (40 mL) was added HBTU (1.915 g, 5.05 mmol). The resulting mixture was stirred under argon at room temperature for 90 minutes. The reaction was concentrated in vacuo, and the residue was partitioned between ethyl acetate and water. The organic phase was washed with water (3×), brine (1×), dried over anhydrous sodium sulfate, filtered and evaporated to give the crude product which was flash chromatographed on silica gel (70 g) eluted with 0-4% methanol in methylene chloride to give the title compound (a mixture of diastereomers) as a white foam. LC-MS M+H⁺=504.

### 61g.) 4((S)-2-Amino-3-cyclopentyl-propanoylamino)-3-hydroxy-azepane-1-carboxylic acid benzyl ester

4((S)-2-tert-Butoxycarbonylamino-3-cyclopentyl-propanoylamino)-3-hydroxy-azepane-1-carboxylic acid benzyl ester (Example 61f, 1.81 g, 3.6 mmol) was dissolved in methanol (55 mL), and treated with HCL in dioxane (4.0 M, 13.5 mL). The mixture was stirred under argon at room temperature for 6 hours. The reaction was concentrated in vacuo. The residue was mixed with toluene and concentrated in vacuo (2×). The residue was triturated with ether (2×), and the residue dried in vacuo overnight to give the crude title product (a mixture of diastereomers) as a white foam which was used without further purification.h LC-MS M+H⁺=404.

### 61h.) 4-{(S)-3-Cyclopentyl-2-[(1-furan-2-yl-methanoyl)-amino]-propanoylamino}-3-hydroxy-azepane-1-carboxylic acid benzyl ester

To a mixture of 4-((S)-2-amino-3-cyclopentyl-propanoylamino)-3-hydroxy-azepane-1-carboxylic acid benzyl ester (Example 61g, 1.6 g, 3.64 mmol), 2-furoic acid (0.416 g, 3.64 mmol), and 4-methylmorpholine (1.84 g, 18.2 mmol) stirring in DMF (46 mL) was added HBTU (1.79 g, 4.73 mmol). The resulting mixture was stirred under argon at room temperature for 1 hour. The reaction was concentrated in vacuo, and the residue partitioned between ethyl acetate and water. The organic phase was washed with water (3×), brine (1×), dried over anhydrous sodium sulfate, filtered and evaporated to give the crude product which was flash chromatographed on silica gel (90 g) eluted with 14% methanol in methylene chloride. This material was rechromatographed on silica gel (120 g) eluted with 0-4% methanol in methylene chloride to give the title compound (a mixture of diastereomers) as a white solid. LC-MS M+H⁺=498.

### 61i.) 4-{(S)-3Cyclopentyl-2-[(1-furan-2-yl-methanoyl)-amino]-propanoylamino}-3-oxo-azepane-1-carboxylic acid benzyl ester

To a solution of 4-{(S)-3-cyclopentyl-2-[(1-furan-2-yl-methanoyl)-amino]-propanoylamino}-3-hydroxy-azepane-1-carboxylic acid benzyl ester (Example 61h, 103 mg, 0.207 mmol) stirring under argon in methylene chloride (10 mL) was added Dess-Martin periodinane (132 mg, 0.311 mmol). The mixture was stirred for 1 hour at room temperature. The reaction was worked up by diluting with methylene chloride and washing the organic phase three times with a 1:1 mixture of 10% NaHCO₃ and 10% Na₂S₂O₅. The organic phase was dried over anhydrous sodium sulfate, filtered and evaporated. The crude product was chromatographed on silica gel (10 g) eluted with 0-4% methanol in methylene chloride to give the title compound as a mixture of diastereomers. LC-MS M+H⁺=496.

### 61j.) 4-{(S)-3-Cyclopentyl-2-[(1-furan-2-yl-methanoyl)-amino]-propanoylamino}-3-oxo-azepane-1-carboxylic acid benzyl ester (first diastereomer eluted)

The mixture of diastereomers from Example 61i was separated on a preparative R,R Whelk-O column. The first diastereomer eluted was the title compound, a white amorphous solid. mp 72-74° C.; LC-MS M+H⁺=496; ¹H NMR (400 Hz, CDCl₃): δ 7.48 (s, 1H), 7.36-7.41 (m, 5H), 6.94-6.99 (m, 1H), 6.78-6.84 (m, 2H), 6.53 (s, 1H), 5.09-5.28 (m, 2H), 4.22-4.82 (m, 4H), 3.62-3.71 (m, 1H), 1.17-2.67 (m, 16H).

### Example 62

### Preparation of 62: 4-{(S)-3-Cyclopentyl-2-[(1-furan-2-yl-methanoyl)-amino]-propanoylamino)-3-oxo-azepane-1-carboxylic acid benzyl ester (second diastereomer eluted)

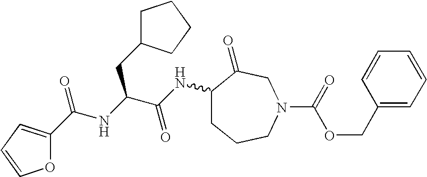

The mixture of diastereomers from Example 61i was separated on a preparative R,R Whelk-O column. The second diastereomer eluted was the title compound, a white amorphous solid. mp 66-68° C.; LC-MS M+H⁺=496; ¹H NMR (400 Hz, CDCl₃): δ 7.49 (s, 1H), 6.74-7.39 (m, 8H), 6.53 (s, 1H), 4.23-4.28 (m, 6H), 3.59-3.70 (m, 1H), 1.18-2.67 (m, 16H).

### Example 63

### Preparation of 63b: Furan-2-carboxylic acid {(S)-2-cyclopentyl-1-[3-oxo-1-(pyridine-2-sulfonyl)-azepan-4-ylcarbamoyl]ethyl}-amide (first diastereomer eluted)

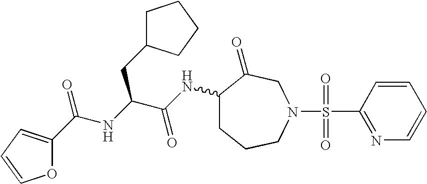

### 63a.) Furan-2-carboxylic acid [(S)-2-cyclopentyl-1-(3-hydroxy-azepan-4-ylcarbamoyl)-ethyl]-amide

4-{(S)-3Cyclopentyl-2-[(1-furan-2-yl-methanoyl)-amino]-propanoylamino}-3-hydroxy-azepane-1-carboxylic acid benzyl ester (Example 61h, 0.5 g, 1 mmol) was dissolved in methylene chloride (10 mL) and stirred under argon in an ice bath. Trimethylsilyl iodide (0.5 mL, 3.5 mmol)was added dropwise, and the ice bath was removed. After stirring for three hours at room temperature the solvent was removed in vacuo. The residue was taken up in ether and extracted three times with IN HCl. The combined aqueous HCl phases were neutralized with solid sodium carbonate, and extracted with methylene chloride (5×). The combined organic phases were dried over anhydrous sodium sulfate, filtered, and evaporated to give the title compound (a mixture of diastereomers) as a white solid which was used without further purification. LC-MS M+H⁺=364.

### 63b.) Furan-2-carboxylic acid {(S)-2-cyclopentyl-1-[3-hydroxy-1-(pyridine-2-sulfonyl)-azepan-4-ylcarbamoyl]-ethyl}-amide

Furan-2-carboxylic acid [(S)-2-cyclopentyl-1-(3-hydroxy-azepan-4-ylcarbamoyl)-ethyl]-amide (Example 63a, 72 mg, 0.2 mmol) was dissolved in methylene chloride (5 mL), and a solution of 10% aqueous sodium bicarbonate (0.84 mL) was added. The mixture was stirred rapidly at room temperature, and pyridine-2-sulfonyl chloride (35.4 mg, 0.2 mmol) was added. After two hours, the reaction was diluted with methylene chloride, and water, and extracted with methylene chloride (3{). The combined organic phases were dried over anhydrous sodium sulfate filtered and evaporated to give the crude product. Flash chromatography on silica gel eluted with 0-4% methanol in methylene chloride gave the title compound as a mixture of diastereomers; C-MS M+H⁺=505.

### 63c.) Furan-2-carboxylic acid {(S)-2-cyclopentyl-1-[3-oxo-1-(pyridine-2-sulfonyl)-azepan-4-ylcarbamoyl]-ethyl}-amide

Following the procedure of Example 61i, except substituting furan-2-carboxylic acid {(S)-2-cyclopentyl-1-[3-hydroxy-1-(pyridine-2-sulfonyl)-azepan-4-ylcarbamoyl]-ethyl}-amide (the product of Example 63b) for 4-{(S)-3-cyclopentyl-2-[(1-furan-2-yl-methanoyl)-amino]-propanoylamino}-3-hydroxy-azepane-1-carboxylic acid benzyl ester. gave the title compound as a mixture of diastereomers. LC-MS M+H⁺=503.

### 63d.) Furan-2-carboxylic acid {(S)-2-cyclopentyl-1-[3-oxo-1-(pyridine-2-sulfonyl)-azepan-4-ylcarbamoyl]-ethyl}-amide (first diastereomer eluted)

The mixture of diastereomers from Example 63c was separated on a preparative Chiralpak AD column. The first diastereomer eluted was the title compound, a white amorphous solid. mp 81-84° C.; LC-MS M+H⁺=503; ¹H NMR (400 Hz, CDCl₃): δ 8.71 (d, 1H), 7.94-7.99 (m, 2H), 7.50-7.54 (m, 2H), 7.16-7.17 (m, 1H), 7.08-7.09 (m, 1H), 6.77-6.80 (m, 1H), 6.53-6.54 (m, 1H), 5.14-5.17 (m, 1H), 4.64-4.76 (m, 2H), 4.11-4.30 (m, 1H), 3.85 (d, 1H), 2.74-2.75 (m, 1H), 1.15-2.25 (m, 15H).

### Example 64

### Preparation of 64: Furan-2-carboxylic acid {(S)-2-cyclopentyl-1-[3-oxo-1-(pyridine-2-sulfonyl)-azepan-4-ylcarbamoyl]ethyl}-amide (second diastereomer eluted)

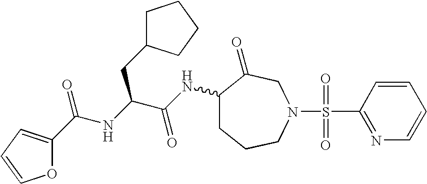

The mixture of diastereomers from Example 63c was separated on a preparative Chiralpak AD column. The second diastereomer eluted was the title compound, a white amorphous solid. mp 77-80° C.; LC-MS M+H⁺=503; ¹H NMR (400 Hz, CDCl₃): δ 8.71 (d, 1H), 7.93-8.01 (m, 2H), 7.48-7.56 (m, 2H), 7.13-7.14 (m, 1H), 6.96-6.97 (m, 1H), 6.84-6.86 (m, 1H), 6.51-6.52 (m, 1H), 5.14-5.22 (m, 1H), 4.64-4.76 (m, 2H), 4.11-4.16 (m, 1H), 3.85 (d, 1H), 2.68-2.75 (m, 1H), 1.19-2.27 (m, 15H).

### Example 65

### Preparation of 65: Furan-2-carboxylic acid [(S)-2-cyclopentyl-1-(1-methanesulfonyl-3-oxo-azepan-4ylcarbamoyl)-ethyl]-amide (first diastereomer eluted)

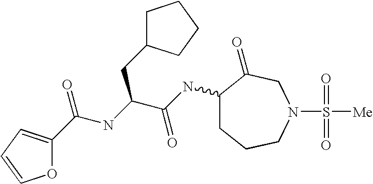

Following the procedure of Example 63(b-d), except substituting methanesulfonyl chloride for pyridine-2-sulfonyl chloride in step 63b, and separating the diastereomers on a preparative R,R Whelk-O column, gave the title compound as the first diastereomer eluted, an off-white amorphous solid. mp 167-170° C.; LC-MS M+H⁺=440; ¹H NMR (400 Hz, CDCl₃): δ 7.48 (s, 1H), 7.15-7.16 (m, 1H), 6.93 (m, 1H), 6.85(m, 1H), 6.51-6.523(m, 1H), 5.14-5.22 (m, 1H), 4.52-4.71 (m, 2H), 4.11-4.16 (m, 1H), 3.65 (d, 1H), 2.93 (s, 3H) 1.16-2.93 (m, 16H).

### Example 66

### Preparation of 66: Furan-2-carboxylic acid [(S)-2-cyclopentyl-1-(1-methanesulfonyl-3-oxo-azepan-4-ylcarbamoyl)-ethyl]-amide (second diastereomer eluted)

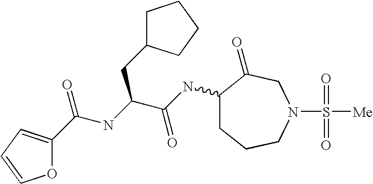

Following the procedure of Example 63(b-d), except substituting methanesulfonyl chloride for pyridine-2-sulfonyl chloride in step 63b, and separating the diastereomers on a preparative R,R Whelk-O column, gave the title compound as the second diastereomer eluted, an off-white amorphous solid. mp 158-161° C.; LC-MS M+H⁺=440; ¹H NMR (400 Hz, CDCl₃): δ 7.49 (s, 1H), 7.16-7.17 (m, 1H), 7.12-7.13 (m, 1H), 6.75-6.78(m, 1H), 6.52-6.54(m, 1H), 5.14-5.22 (m, 1H), 4.48-4.70 (m, 2H), 4.01-4.06 (m, 1H), 3.68 (d, 1H), 2.92 (s, 3H) 1.15-2.82 (m, 16H).

### Example 67

### Preparation of 67: Furan-2-carboxylic acid [(S)-1-(1-benzenesulfonyl-3-oxo-azepan-4 ylcarbamoyl)-2-cyclopentyl-ethyl]-amide (first diastereomer eluted)

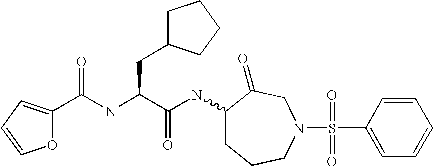

Following the procedure of Example 63(b-d), except substituting benzenesulfonyl chloride for pyridine-2-sulfonyl chloride in step 63b, and separating the diastereomers on a preparative R,R Whelk-O column, gave the title compound as the first diastereomer eluted, an off-white amorphous solid. mp 88-90° C.; LC-MS M+H⁺=502; ¹H NMR (400 Hz, CDCl₃): δ 7.82 (d, 2H), 7.48-7.66 (m, 4H), 7.13-7.14 (m, 1H), 6.82-6.91 (m, 2H), 6.52-6.53(m, 1H), 5.05-5.09 (m, 1H), 4.59-4.63 (m, 2H), 4.04-4.07 (m, 1H), 3.45 (d, 1H), 1.19-2.51 (m, 16H).

### Example 68

### Preparation of 68: Furan-2-carboxylic acid [(S)-1-(1-benzenesulfonyl-3-oxo-azepan-4-ylcarbamoyl)-2-cyclopentyl-ethyl]-amide (second diastereomer eluted)

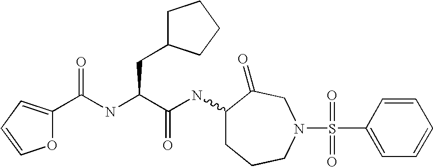

Following the procedure of Example 63(b-d), except substituting benzenesulfonyl chloride for pyridine-2-sulfonyl chloride in step 63b, and separating the diastereomers on a preparative R,R Whelk-O column, gave the title compound as the second diastereomer eluted, a white crystalline solid. mp 166-167° C.; LC-MS M+H⁺=502; ¹H NMR (400 Hz, CDCl₃): δ 7.80 (d, 2H), 7.50-7.66 (m, 4H), 7.17-7.18 (m, 1H.), 7.04 (m, 1H), 6.78 (m, 1H), 6.53-6.54(m, 1H), 5.03-5.08 (m, 1H), 4.50-4.66 (m, 2H), 3.98-4.02 (m, 1H), 3.48 (d, 1H), 1.18-2.56 (m, 16H).

### Example 69

### Preparation of 69: Furan-2-carboxylic acid {(S)-2-cyclopentyl-1-[1-(1-furan-2-yl-methanoyl)-3-oxo-azepan-4-ylcarbamoyl]-ethyl}-amide (first diastereomer eluted)

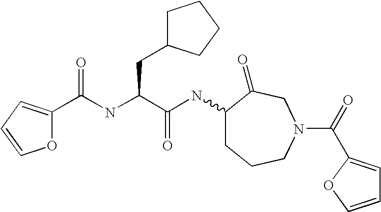

### 69a.) Furan-2-carboxylic acid {(S)-2-cyclopentyl-1-[1-(1-furan-2-yl-methanoyl)-3-hydroxy-azepan-4-ylcarbamoyl]-ethyl}-amide

To a mixture of furan-2-carboxylic acid [(S)-2-cyclopentyl-1-(3-hydroxy-azepan-4-ylcarbamoyl)-ethyl]-amide (Example 63a,70 mg, 0.19 mmol), 2-furoic acid (22.4 mg, 0.19 mmol), and 4-methylmorpholine (0.1 mL, 0.95 mmol) stirring in DMF (2 mL) was added HBTU (93 mg, 0.25 mmol). The resulting mixture was stirred under argon at room temperature for 80 minutes. The reaction was concentrated in vacuo, and the residue was partitioned between ethyl acetate and water. The organic phase was washed with water (4×), brine (1×), dried over anhydrous sodium sulfate, filtered and evaporated to give the crude product which was flash chromatographed on silica gel (10 g) eluted with 0-5% methanol in methylene chloride to give the title compound as a mixture of diastereomers. LC-MS M+H⁺=458.

### 69b.) Furan-2-carboxylic acid {(S)2-cyclopentyl-1-[1-(1-furan-2-yl-methanoyl)-3-oxo-azepan-4-ylcarbamoyl]-ethyl}-amide

Following the procedure of Example 61i,_except substituting furan-2-carboxylic acid {(S)-2-cyclopentyl-1-[1-(1-furan-2-yl-methanoyl)-3-hydroxy-azepan-4-ylcarbamoyl]-ethyl}-amide (the product of Example 69a) for 4-{(S)-3-cyclopentyl-2-[(1-furan-2-yl-methanoyl)-amino-propanoylamino}-3-hydroxy-azepane-1-carboxylic acid benzyl ester. gave the title compound as a mixture of diastereomers. LC-MS M+H⁺=456.

### 69c.) Furan-2-carboxylic acid {(S)2-cyclopentyl-1-[1-(1-furan-2-yl-methanoyl)-3-oxo-azepan-4-ylcarbamoyl]-ethyl}-amide (first diastereomer eluted)

The mixture of diastereomers from Example 69b was separated on a preparative R,R Whelk-O column. The first diastereomer eluted, an off-white amorphous solid, was the title compound. mp 88-89° C.; LC-MS M+H⁺=456; ¹H NMR (400 Hz, CDCl₃): δ 7.48-7.55 (m, 2H), 7.14-7.24 (m, 2H), 6.98 (m, 1H), 6.80 (m, 1H), 6.52-6.55(m, 2H), 5.4 (m, 1H), 4.60-4.90 (m, 4H), 3.70 (m, 1H), 1.18-2.30 (m, 15H).

### Example 70

### Preparation of 70: Furan-2-carboxylic acid {(S)-2-cyclopentyl-1-[1-(1-furan-2-yl-methanoyl)-3-oxo-azepan-4-ylcarbamoyl]-ethyl}-amide (second diastereomer eluted)

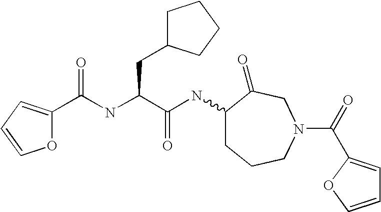

The mixture of diastereomers from Example 69b was separated on a preparative R,R Whelk-O column. The second diastereomer eluted, an off-white amorphous solid, was the title compound. mp 85-88° C.; LC-MS M+H⁺=456; ¹H NMR (400 Hz, CDCl₃): δ 7.47-7.52 (m, 2H), 7.14-7.19 (m, 3H), 6.75-6.76 (m, 1H), 6.51-6.53 (m, 2H), 5.20-5.30 (m, 1H), 4.61-4.67 (m, 4H), 3.65-3.95 (m, 1H), 1.18-3.05 (m, 15H).

### Example 71

### Preparation of 71: Furan-2-carboxylic acid {(S)-2-cyclopentyl-1-[3-oxo-1-(1-phenyl-methanoyl)-azepan-4-ylcarbamoyl]-ethyl}-amide (first diastereomer eluted)

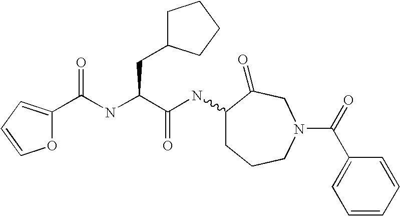

Following the procedure of Example 69(a-c) except substituting benzoic acid for furan-2-carboxylic acid in step 69a gave the title compound, as the first diastereomer eluted. mp 101-103° C.; LC-MS M+H⁺=466; ¹H NMR (400 Hz, CDCl₃): δ 7.48 (m, 5H), 7.14-7.15 (m, 1H), 6.75-6.76 (m, 1H), 7.03 (m, 1H), 6.83 (m, 1H) 6.52-6.53 (m, 1H) 5.30-5.40 (m, 1H), 3.64-4.79 (m, 5H), 1.18-3.05 (m, 15H).

### Example 72

### Preparation of 72: Furan-2-carboxylic acid {(S)-2-cyclopentyl-1-[3-oxo-1-(1-phenyl-methanoyl)-azepan-4-ylcarbamoyl]-ethyl}-amide (second diastereomer eluted)

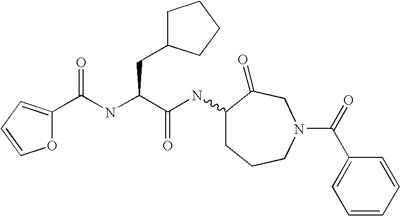

Following the procedure of Example 69(a-c) except substituting benzoic acid for furan-2-carboxylic acid in step 69a gave the title compound, as the second diastereomer eluted. mp 97-100° C.; LC-MS M+H⁺=466; ¹H NMR (400 Hz, CDCl₃): δ 7.20-7.45 (m, 6H), 7.14-7.15 (m, 1H), 7.03 (m, 1H), 6.82-6.84 (m, 1H) 6.51-6.52 (m, 1H) 5.20-5.40 (m, 1H), 3.64-4.90 (m, 5H), 1.18-3.05 (m, 15H).

### Example 73

### Preparation of 73: Piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide

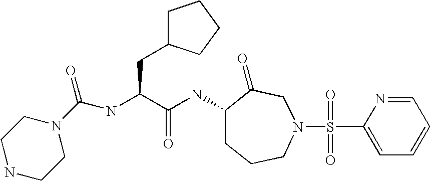

Following the general procedure described in Example 37c, (S)-2-amino-3-cyclohexyl-N-[(3S,4S)-3-hydroxy-1-(pyridine-2-sulfonyl)-azepan-4-yl]-propionamide HCl salt (Example 38g, 150 mg, 0.33 mmol) was coupled with 4-(1-imidazol-1-yl-methanoyl)-piperazine-1-carboxylic acid tert-butyl ester methyl iodide salt (Example 37b, 139 mg, 0.33 mmol) to give the 3-hydroxy intermediate. Upon oxidation with Dess-Martin periodinane (182 mg, 0.43 mmol) followed by removal of the tert-butoxycarbonyl protecting group with 4N HCl the title compound was obtained (8 mg, 4%). LC-MS m/z 535.2 (M⁺), 1.45 min.

### Example 74

### Preparation of 74A: morpholine 4-carboxylic acid {(S)-2-[1-methylcyclohexyl-1-(4S,7R)-7-methyl-3-oxo-1-(1-pyridin-2-yl-meyhanoyl)-azepan-4-ylcarbamoyl]-ethyl}-amide

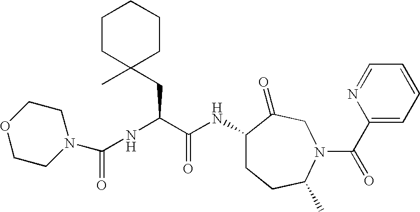

### Preparation of 74B: morpholine 4-carboxylic acid {(L)-2-[1-methylcyclohexyl-1-(4S,7R)-7-methyl-3-oxo-1-(1-pyridin-2-yl-methanoyl)-azepan-4-ylcarbamoyl]-ethyl}-amide

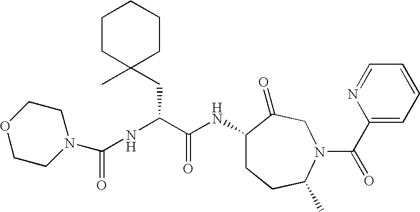

Following the procedure of Example 1(b-r), except substituting “1-pyridin-2-yl-methanoyl” for “1-oxy-pyridine-2-sulfonyl” gave the title compound: ¹HNMR data of 74A: ¹H NMR (400 Hz, CDCl₃): δ 8.4 (d, 1H), 7.65 (m, 2H), 7.35 (m, 1H), 6.95 (d, 1H), 5.35 (m, 1H), 4.97 (m, 2H), 4.55 (d, 1H), 4.45 (m, 1H), 3.80 (d, 1H), 3.70 (t, 4H), 3.35 (t, 4H), 2.4 (m, 1H), 2.15 (m, 1H), 0.95-1.9 (m, 18H). The ¹H NMR data of 75B:¹H NMR (400 Hz, CDCl₃): δ 8.5 (d, 1H), 7.82 (m, 2H), 7.35 (m, 1H), 7.1 (d, 1H), 5.25 (m, 1H), 4.97 (m, 2H), 4.6 (d, 1H), 4.45 (m, 1H), 3.80 (d, 1H), 3.70 (t, 4H), 3.35 (t, 4H), 2.4 (m, 1H), 2.15 (m, 1H), 0.95-1.9 (m, 18H).

### Example 75

### Preparation of 75A: 2-Methyl-thiazole-4-carboxylic acid {(S)-2-[1-methylcyclohexyl-1-(4S,7R)-7-methyl-3-oxo-1-(1-pyridin-2-yl-meyhanoyl)-azepan-4-ylcarbamoyl]-ethyl}-amide

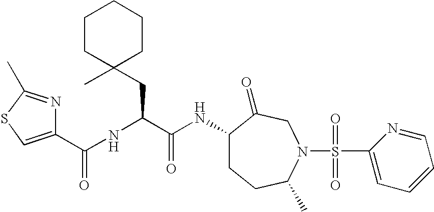

### Preparation of 74B: 2-Methyl-thiazole-4-carboxylic acid {(L)-2-[1-methylcyclohexyl-1-(4S,7R)-7-methyl-3-oxo1-(1-pyridin-2-yl-methanoyl)-azepan-4-ylcarbamoyl]-ethyl}-amide

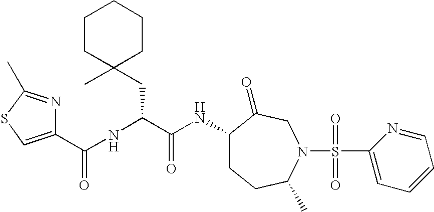

Following the procedure of Example 3(f-r), except substituting “2-Methyl-thiazole-4-carboxylic acid” for “morpholine-4-carboxylic acid” gave the title compound: ¹HNMR data of 74A: ¹H NMR (400 Hz, CDCl₃): δ 8.7 (d, 1H), 8.08 (d, 1H), 7.92 (s, 1H), 7.6 (d, 1H), 7.54 (d, 1H), 6.88 (d, 1H), 5.1 (m, 1H), 4.6 (m, 1H), 4.2 (d, 1H), 4.0 (m, 2H), 3.8 (m, 1H), 3.4 (d, 1H), 2.7 (s, 3H), 2.2 (m, 2H), 0.9-1.7 (m, 19H). The ¹H NMR data of 75B: ¹H NMR (400 Hz, CDCl₃): δ 8.7 (d, 1H), 8.1 (d, 1H), 7.92 (s, 1H), 7.65 (d, 1H), 7.52 (d, 1H), 6.9 (d, 1H), 5.10 (m, 1H), 4.6 (m, 1H), 4.1 (d, 1H), 4.0 (m, 2H), 3.8 (m, 1H), 3.4 (d, 1H), 2.7 (s, 3H), 2.2 (m, 2H), 0.9-1.7 (m, 19H).

1. A method for treating a disease by inhibiting cathepsin S comprising administering at least one compound of Formula I neat or in a pharmaceutically acceptable formulation in an effective amount to a mammal in need thereof, wherein Formula I comprises: 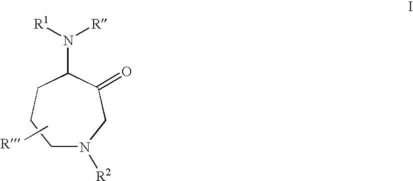
wherein:
R¹ is 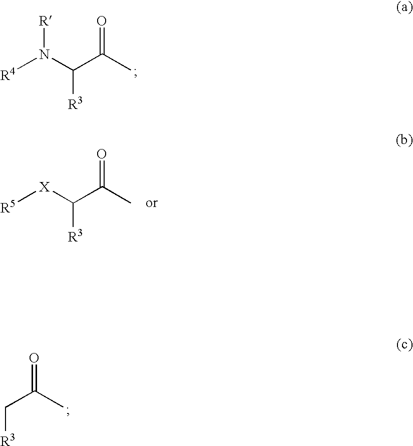
R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂— 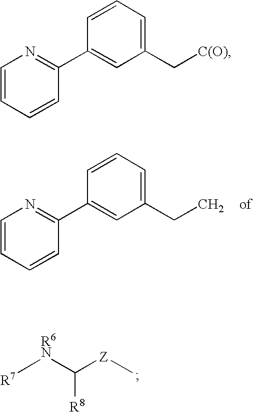
R³ is H or substituted or unsubstituted C₁₋₆alkyl, C₃₋₇cycloalkylC₀₋₆alkyl, C₄₋₇cycloalkenylC₀₋₆alkyl, C₅₋₈bicycloalkylC₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, or Het-HetC₀₋₆alkyl;
R⁴ is H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵NSO₂—, R⁵OC(O)—, R⁵R¹²NC(O)—, or R⁵R¹²NC(S)—;
R⁵ is H, C₁₋₆alkyl, C₂₋₆alkenyl, C₂₋₆alkynyl, C₃₋₆cycloalkyl-C₀₋₆alkyl, Ar—C₀₋₆alkyl, Ar—ArC₀₋₆alkyl, Ar-HetC₀₋₆alkyl, Het-ArC₀₋₆alkyl, Het-HetC₀₋₆alkyl, or Het-C₀₋₆alkyl;
R⁶ is H, C₁₋₆alkyl, C₃₋₆cycloalkyl-C₀₋₆alkyl, Ar—C₀₋₆alkyl, or Het-C₀₋₆alkyl;
R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, or R¹⁰R¹³NC(S)—;
R⁸ is H, C₁₋₆alkyl, C₂₋₆alkenyl, C₂₋₆alkynyl, HetC₀₋₆alkyl or ArC₀₋₆alkyl;
R⁹ is C₁₋₆alkyl, C₃₋₆cycloalkyl-C₀₋₆alkyl, Ar—C₀₋₆alkyl or Het-C₀₋₆alkyl;
R¹⁰ is C₁₋₆alkyl, C₃₋₆cycloalkyl-C₀₋₆alkyl, Ar—C₀₋₆alkyl or Het-C₀₋₆alkyl;
R¹¹ is H, C₁₋₆alkyl, Ar—C₀₋₆alkyl, or Het-C₀₋₆alkyl;
R¹² is H, C₁₋₆alkyl, Ar—C₀₋₆alkyl, or Het-C₀₋₆alkyl;
R¹³ is H, C₁₋₆alkyl, Ar—C₀₋₆alkyl, or Het-C₀₋₆alkyl;
R′ is H, C₁₋₆alkyl, Ar—C₀₋₆alkyl, or Het-C₀₋₆alkyl;
R″ is H, C₁₋₆alkyl, Ar—C₀₋₆alkyl, or Het-C₀₋₆alkyl;
R′″ is C₁₋₆alkyl, C₃₋₆cycloalkyl-C₀₋₆alkyl, Ar—C₀₋₆alkyl, or Het-C₀₋₆alkyl;
X is CH₂, S, or O;
Z is C(O) or CH₂; or
a pharmaceutically acceptable salt, hydrate or solvate thereof.

2. The method of claim 1 wherein, in Formula I:
R¹ is 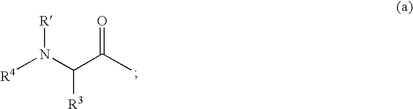
R² is R⁹SO₂ R⁹OC(O)—, or R⁹C(O)—;
R³ is C₅₋₇cycloalkylC₁₋₂alkyl, C₄₋₅cycloalkenylC₁₋₂alkyl, C₅₋₈bicycloalkylC₁₋₂alkyl or Ar-HetC₀₋₆alkyl;
R⁴ is R⁵C(O)—, or R⁵SO₂—;
R⁵ is unsubstituted or substituted furanyl, tetrahydrofuranyl, morpholinyl, pyrrolyl, piperazinyl, pyrazolyl, isoxazolyl, thiazolyl, pyrazolyl, pyrazolo[5,1-c]pyrimidinyl, triazolyl, pyrazinyl, imadazolyl, benzofuranyl, thiophenyl, furo[3,2-b]-pyridine-2-yl, phenyl, pyridinyl, thieno[3,2-b]thiopheneyl, or unsubsituted or C₁₋₂alkylsubstituted pyrazolo[5,1-c]triazinyl;
R⁹ is HetC₀₋₆alkyl, ArC₀₋₆alkyl, or C₁₋₆alkyl;
R′ is H, C₁₋₆alkyl;
R″ is H or C₁₋₆alkyl; and
R′″ is C₁₋₆alkyl.

3. The method of claim 1 wherein in Formula I,
R¹ is 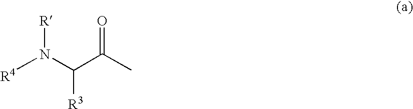
R² is R⁹SO₂R⁹OC(O)—, or R⁹C(O)—;
R³ is cyclopentylmethyl, cyclopentylethyl, cyclopentenylmethyl, cyclopentenylethyl, cyclohexylmethyl, 4-methylcyclohexylmethyl, 2-cyclohexylprop-1-yl, cyclohexylethyl, cycloheptylmethyl, 7,7-dimethylbicyclo[2.2.1]hept-1-ylmethyl, or indol-2-ylmethyl;
R⁴ is R⁵C(O)— or R⁵SO₂—
R⁵ is furan-2-yl, furan-3-yl, 2-methylfuran-3-yl, 2,4-dimethylfuran-3-yl, 5-phenylfuran-2-yl, 5-(2-chlorophenyl)furan-2-yl, 5-(3-chlorophenyl)furan-2-yl, 5-(4-chlorophenyl)furan-2-yl, 5-(4-fluorophenyl)furan-2-yl, 5-(4-hydroxyphenyl)furan-2-yl, 5-(3-trifluoromethylphenyl)furan-2-yl, 5-(4-trifluoromethylphenyl)furan-2-yl, 5-(3-trifluoromethylphenyl)furan-2-yl, 5-(4-methylphenyl)furan-2-yl, 5-(4-acetylphenyl)furan-2-yl, or 5-trifluoromethylfuran-2-yl;
tetrahydrofuran-2-yl or tetrahydrofuran-3-yl
N-morpholinyl;
pyrrol-2-yl;
piperzin-1-yl or 4-methylpeperzin-1-yl;
1H-pyrazol-2-yl, 1H-pyrazol-4-yl, 1-methyl-2H-pyrazol-2-yl, 2-methyl-2H-pyrazol-2-yl, 1-methyl-2H-pyrazol-3-yl or 2-methyl-2H-pyrazol-3-yl;
isoxazol-5-yl, 3-methylisoxazol-4-yl, 5-methylisoxazol-3-yl, 5-methylisoxazol-4-yl, or 3,5-dimethylisoxazol-4-yl;
thiazol-2-yl, 2-methylthiazol-2-yl, 2,4-dimethylthiazol-5-yl, 2-(2,3-dihydrobenzo[1,4]dioxin-2-yl)thiazol-4-yl, or 4-methyl-2-phenylthiazol-5-yl;
4,7-dimethylpyrazolo[5,1-c]triazin-3-yl;
2-methyl-2H-pyrazol-2-yl;
2,7-dimethylpyrazol[5,1-c]pyrimidin-6-yl;
3-phenyl-3H-{1,2,3]triazol-3-yl;
pyrazin-2-yl or 5-methylpyrazin-2-yl;
1-H-imidazol-2-yl, 1-methyl-1H-imidazol-4-yl or 1-methyl-1H-imidazol-2-yl;
benzofuran-2-yl, 5,6-dimethoxybenzofuran-2-yl, or 5-(2-morpholin-4-yl-ethoxy)benzofuran-2-yl;
thiophene-3-yl, or thiophen-2-yl, 5-pyridin-2-ylthiophen-2-yl, 5-methylthiophenyl 3-methylthiophen-2-yl; or 3-ethoxythiophen-2-yl;
furo[3,2-b]-pyridine-2-yl or 3-methylfuro[3,2-b]pyridin-2-yl;
phenyl, 4-methylphenyl, 3-chlorophenyl, 4-chlorophenyl, 3-trifluoromethylphenyl, 4-trifluoromethylphenyl, 2-chlorophenyl, 4-fluorophenyl, 4-hydroxyphenyl, or 4-acetylphenyl;
pyridin-2-yl; or
thieno[3,2-b]thiophen-2-yl or 5-isoxazol-3-ylthiophen-2-yl;
R⁹ is pyridin-2-yl, 1-oxypyridin-2-yl, phenyl, furan-2-yl or methyl;
R′ is H;
R″ is H or C₁₋₆alkyl; and
R′″ is methyl.

4. The method according to claim 1 wherein Formula I has the structure: 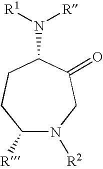
and groups R¹-R¹³, X, Z, R′, R″ and R′″ are the same as defined in claim 1.

5. The method according to claim 1 wherein the compound of Formula I is
morpholine 4-carboxylic acid {(S)-2-[1-methylcyclopentyl-1-(4S,7R)-7-methyl-3-oxo-1-(1-oxy-pyridine-2-sulfonyl)-azepan-4-ylcarbamoyl]-ethyl}-amide;
morpholine 4-carboxylic acid {(L)-2-[1-methylcyclopentyl-1-(4S,7R)-7-methyl-3-oxo-1-(1-oxy-pyridine-2-sulfonyl)-azepan-4-ylcarbamoyl]-ethyl}-amide;
morpholine 4-carboxylic acid {(S)-2-[1-methylcyclohexyl-1-(4S,7R)-7-methyl-3-oxo-1-(1-oxy-pyridine-2-sulfonyl)-azepan-4-ylcarbamoyl]-ethyl}-amide;
morpholine 4-carboxylic acid {(L)-2-[1-methylcyclohexyl-1-(4S,7R)-7-methyl-3-oxo-1-(1-oxy-pyridine-2-sulfonyl)-azepan-4-ylcarbamoyl]-ethyl}-amide;
furan-carboxylic acid {(S)-2-[1-methylcyclohexyl-1-(4S,7R)-7-methyl-3-oxo-1-(1-oxy-pyridine-2-sulfonyl)-azepan-4-ylcarbamoyl]-ethyl}-amide;
furan-carboxylic acid {(L)-2-[1-methylcyclohexyl-1-(4S,7R)-7-methyl-3-oxo-1-(1-oxy-pyridine-2-sulfonyl)-azepan-4-ylcarbamoyl]-ethyl}-amide;
furan-carboxylic acid {(S)-2-[4-methylcyclohexyl-1-(4S,7R)-7-methyl-3-oxo-1-(1-oxy-pyridine-2-sulfonyl)-azepan-4-ylcarbamoyl]-ethyl}-amide;
furan-carboxylic acid {(S)-2-[homocyclopentyl-1-(4S,7R)-7-methyl-3-oxo-1-(1-oxy-pyridine-2-sulfonyl)-azepan-4-ylcarbamoyl]-ethyl}-amide;
morpholine 4-carboxylic acid {(S)-2-[homocyclopentyl-1-(4S,7R)-7-methyl-3-oxo-1-(1-oxy-pyridine-2-sulfonyl)-azepan-4-ylcarbamoyl]-ethyl}-amide;
furan-carboxylic acid {(S)-2-[cycloheptyl-1-(4S,7R)-7-methyl-3-oxo-1-(1-oxy-pyridine-2-sulfonyl)-azepan-4-ylcarbamoyl]-ethyl}-amide;
morpholine 4-carboxylic acid {(S)-2-[cycloheptyl-1-(4S,7R)-7-methyl-3-oxo-1-(1-oxy-pyridine-2-sulfonyl)-azepan-4-ylcarbamoyl]-ethyl}-amide;
furan-carboxylic acid {(S)-2-[cyclopentenyl-1-(4S,7R)-7-methyl-3-oxo-1-(1-oxy-pyridine-2-sulfonyl)-azepan-4-ylcarbamoyl]-ethyl}-amide;
furan-carboxylic acid {(S)-2-[tryptophanyl-1-(4S,7R)-7-methyl-3-oxo-1-(1-oxy-pyridine-2-sulfonyl)-azepan-4-ylcarbamoyl]-ethyl}-amide;
morpholine 4-carboxylic acid {(S)-2-(7,7-dimethyl-bicyclo[2.2.1]hepty-1-yl)-1-[(4S,7R)-7-methyl-3-oxo-1-(1-oxy-pyridine-2-sulfonyl)-azepan-4-ylcarbamoyl]-ethyl}-amide;
2-methyl-2H-pyrazole-3-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
1H-pyrazole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
1-methyl-1H-pyrrole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
isoxazole-5-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
thiazole-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-trifluoromethyl-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
1H-pyrazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
tetrahydrofuran-3-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
4,7-dimethyl-pyrazolo[5,1-c][1,2,4]triazine-3-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
2,7-dimethyl-pyrazolo[5,1-a]pyrimidine-6-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
3-phenyl-3H-[1,2,3]triazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
2-(2,3-dihydro-benzo[1,4]dioxin-2-yl)-thiazole-4-carboxylic acid {(S)-2-cyclopentyl-1-[(4S,7R)-7-methyl-3-oxo-1-(pyridine-2-sulfonyl)-azepan-4-ylcarbamoyl]-ethyl}-amide
N-{(S)-2-cyclopentyl-1-[(4S,7R)-7-methyl-3-oxo-1-(pyridine-2-sulfonyl)-azepan-4-ylcarbamoyl]-ethyl}-2-pyrazol-1-yl-benzamide;
4-methyl-2-phenyl-thiazole-5-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(4-chloro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(3-trifluoromethyl-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(2-chloro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(4-fluoro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(4-methoxy-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-phenyl-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(4-trifluoromethyl-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(3-chloro-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(4-methylphenyl)furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
5-(4-acetyl-phenyl)-furan-2-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
4-methyl-piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide;
morpholine 4-carboxylic acid {(S)-2-[1-methylcyclohexyl-1-(4S,7R)-7-methyl-3-oxo-1-(1-pyridin-2-yl-meyhanoyl)-azepan-4-ylcarbamoyl]-ethyl}-amide;
morpholine 4-carboxylic acid {(L)-2-[1-methylcyclohexyl-1-(4S,7R)-7-methyl-3-oxo-1-(1-pyridin-2-yl-methanoyl)-azepan-4-ylcarbamoyl]-ethyl}-amide;
2-methyl-thiazole-4-carboxylic acid {(S)-2-[1-methylcyclohexyl-1-(4S,7R)-7-methyl-3-oxo-1-(1-pyridin-2-yl-meyhanoyl)-azepan-4-ylcarbamoyl]-ethyl}-amide;
2-methyl-thiazole-4-carboxylic acid {(L)-2-[1-methylcyclohexyl-1-(4S,7R)-7-methyl-3-oxo-1-(1-pyridin-2-yl-methanoyl)-azepan-4-ylcarbamoyl]-ethyl}-amide;
morpholine 4-carboxylic acid {(S)-2-[1-methylcyclohexyl-1-(4S,7R)-7-methyl-3-oxo-1-(1-pyridin-2-yl-meyhanoyl)-azepan-4-ylcarbamoyl]-ethyl}-amide;
morpholine 4-carboxylic acid {(L)-2-[1-methylcyclohexyl-1-(4S,7R)-7-methyl-3-oxo-1-(1-pyridin-2-yl-methanoyl)-azepan-4-ylcarbamoyl]-ethyl}-amide;
2-methyl-thiazole-4-carboxylic acid {(S)-2-[1-methylcyclohexyl-1-(4S,7R)-7-methyl-3-oxo-1-(1-pyridin-2-yl-meyhanoyl)-azepan-4-ylcarbamoyl]-ethyl}-amide;
2-methyl-thiazole-4-carboxylic acid {(L)-2-[1-methylcyclohexyl-1-(4S,7R)-7-methyl-3-oxo-1-(1-pyridin-2-yl-methanoyl)-azepan-4-ylcarbamoyl]-ethyl}-amide; or a pharmaceutically acceptable salt, hydrate or solvate thereof.

6. The method of claim 1 wherein the inhibition of cathepsin S effects treatment or prevention of an autoimmune disease; treatment or prevention of a disease caused by the formation of atherosclerotic lesions and complications arising therefrom; and diseases requiring inhibition, for therapy, of a class II MHC-restricted immune response, inhibition of an asthmatic response, inhibition of an allergic response, inhibition of immune response against a transplanted organ or tissue, or inhibition of elastase activity in atheroma.

7. A compound of Formula II 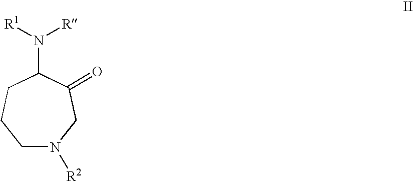
wherein:
R¹ is: 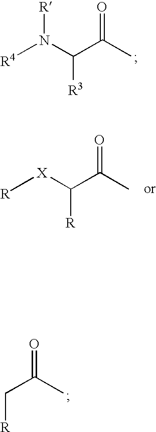
R² is H, C₁₋₆alkyl, C₃₋₆cycloalkyl-C₀₋₆alkyl, Ar—C₀₋₆alkyl, Het-C₀₋₆alkyl, R⁹C(O)—, R⁹C(S)—, R⁹SO₂—, R⁹OC(O)—, R⁹R¹¹NC(O)—, R⁹R¹¹NC(S)—, R⁹(R¹¹)NSO₂— 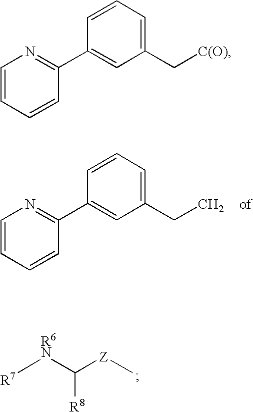
R³ is H or substituted or unsubstituted C₁₋₆alkyl, C₃₋₇cycloalkylC₀₋₆alkyl, C₄₋₇cycloalkenylC₀₋₆alkyl, C₅₋₈bicycloalkylC₀₋₆alkyl, C₂₋₆alkenyl, C₂₋₆alkynyl, HetC₀₋₆alkyl, ArC₀₋₆alkyl, Ar—ArC₀₋₆alkyl, Ar-HetC₀₋₆alkyl, Het-ArC₀₋₆alkyl, or Het-HetC₀₋₆alkyl;
R⁴ is H, C₁₋₆alkyl, C₃₋₆cycloalkyl-C₀₋₆alkyl, Ar—C₀₋₆alkyl, Het-C₀₋₆alkyl, R⁵C(O)—, R⁵C(S)—, R⁵SO₂—, R⁵NSO₂—, R⁵OC(O)—, R⁵R¹²NC(O)—, or R⁵R¹²NC(S)—;
R⁵ is H, C₁₋₆alkyl, C₂₋₆alkenyl, C₂₋₆alkynyl, C₃₋₆cycloalkyl-C₀₋₆alkyl, Ar—C₀₋₆alkyl, Ar—ArC₀₋₆alkyl, Ar-HetC_{0 6}alkyl, Het-ArC₀₋₆alkyl, Het-HetC₀₋₆alkyl, or Het-C₀₋₆alkyl;
R⁶ is H, C₁₋₆alkyl, C₃₋₆cycloalkyl-C₀₋₆alkyl, Ar—C₀₋₆alkyl, or Het-C₀₋₆alkyl;
R⁷ is H, C₁₋₆alkyl, C₃₋₆cycloalkyl-C₀₋₆alkyl, Ar—C₀₋₆alkyl, Het-C₀₋₆alkyl, R¹⁰C(O)—, R¹⁰C(S)—, R¹⁰SO₂—, R¹⁰OC(O)—, R¹⁰R¹³NC(O)—, or R¹⁰R¹³NC(S)—;
R⁸ is H, C₁₋₆alkyl, C₂₋₆alkenyl, C₂₋₆alkynyl, HetC₀₋₆alkyl or ArC₀₋₆alkyl;
R⁹ is C₁₋₆alkyl, C₃₋₆cycloalkyl-C₀₋₆alkyl, Ar—C₀₋₆alkyl or Het-C₀₋₆alkyl;
R¹⁰ is C₁₋₆alkyl, C₃₋₆cycloalkyl-C₀₋₆alkyl, Ar—C₀₋₆alkyl or Het-C₀₋₆alkyl;
R¹¹ is H, C₁₋₆alkyl, Ar—C₀₋₆alkyl, or Het-C₀₋₆alkyl;
R¹² is H, C₁₋₆alkyl, Ar—C₀₋₆alkyl, or Het-C₀₋₆alkyl;
R¹³ is H, C₁₋₆alkyl, Ar—C₀₋₆alkyl, or Het-C₀₋₆alkyl;
R′ is H, C₁₋₆alkyl, Ar—C₀₋₆alkyl, or Het-C₀₋₆alkyl;
R″ is C₁₋₆alkyl, Ar—C₀₋₆alkyl, or Het-C₀₋₆alkyl;
X is CH₂, S, or O;
Z is C(O) or CH₂; or
a pharmaceutically acceptable salt, hydrate or solvate thereof.

8. A compound of Formula I according to claim 7 wherein:
R¹ is 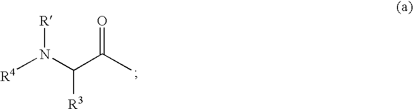
R² is R⁹SO₂, R⁹OC(O)—, R⁹C(O)— or C₁₋₆alkyl;
R³ is C₅₋₇cycloalkylC₁₋₂alkyl, C₄₋₅cycloalkenylC₁₋₂alkyl, C₅₋₈bicycloalkylC₁₋₂alkyl or Ar-HetC₀₋₆alkyl;
R⁴ is R⁵C(O)—, or R⁵SO₂—;
R⁵ is unsubstituted or substituted furanyl, tetrahydrofuranyl, morpholinyl, pyrrolyl, piperazinyl, pyrazolyl, isoxazolyl, thiazolyl, pyrazolyl, pyrazolo[5,1-c]pyrimidinyl, triazolyl, pyrazinyl, imadazolyl, benzofuranyl, thiophenyl, furo[3,2-b]-pyridine-2-yl, phenyl, pyridinyl, thieno[3,2-b]thiopheneyl, or unsubsituted or C₁₋₂alkylsubstituted pyrazolo[5,1-c]triazinyl;
R⁹ is Het-C₀₋₆alkyl, ArC₀₋₆alkyl or C₁₋₆alkyl;
R′ is H, C₁₋₆alkyl; and
R″ is H or C₁₋₆alkyl.

9. A compound of Formula II according to claim 8 wherein:
R¹ is 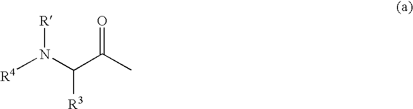
R² is R⁹SO₂ R⁹OC(O)—, or R⁹C(O)—;
R³ is cyclopentylmethyl, cyclopentylethyl, cyclopentenylmethyl, cyclopentenylethyl, cyclohexylmethyl, 4-methylcyclohexylmethyl, 2-cyclohexylprop-1-yl, cyclohexylethyl, cycloheptylmethyl, 7,7-dimethylbicyclo[2.2.1]hept-1ylmethyl, or indol-2-ylmethyl;
R⁴ is R⁵C(O)— or R⁵SO₂—
R⁵ is furan-2-yl, furan-3-yl, 2-methylfuran-3-yl, 2,4-dimethylfuran-3-yl, 5-phenylfuran-2-yl, 5-(2-chlorophenyl)furan-2-yl, 5-(3-chlorophenyl)furan-2-yl, 5-(4-chlorophenyl)furan-2-yl, 5-(4-fluorophenyl)furan-2-yl, 5-(4-hydroxyphenyl)furan-2-yl, 5-(3-trifluoromethylphenyl)furan-2-yl, 5-(4-trifluoromethylphenyl)furan-2-yl, 5-(3-trifluoromethylphenyl)furan-2-yl, 5-(4-methylphenyl)furan-2-yl, 5-(4-acetylphenyl)furan-2-yl, or 5-trifluoromethylfuran-2-yl;
tetrahydrofuran-2-yl or tetrahydrofuran-3-yl
N-morpholinyl;
pyrrol-2-yl;
piperzin-1-yl or 4-methylpeperzin-1-yl;
1H-pyrazol-2-yl, 1H-pyrazol-4-yl, 1-methyl-2H-pyrazol-2-yl, 2-methyl-2H-pyrazol-2-yl, 1-methyl-2H-pyrazol-3-yl or 2-methyl-2H-pyrazol-3-yl;
isoxazol-5-yl, 3-methylisoxazol-4-yl, 5-methylisoxazol-3-yl, 5-methylisoxazol-4-yl, or 3,5-dimethylisoxazol-4-yl;
thiazol-2-yl, 2-methylthiazol-2-yl, 2,4-dimethylthiazol-5-yl, 2-(2,3-dihydrobenzo[1,4]dioxin-2-yl)thiazol-4-yl, or 4-methyl-2-phenylthiazol-5-yl;
4,7-dimethylpyrazolo[5,1-c]triazin-3-yl;
2-methyl-2H-pyrazol-2-yl;
2,7-dimethylpyrazol[5,1-c]pyrimidin-6-yl;
3-phenyl-3H-{1,2,3]triazol-3-yl;
pyrazin-2-yl or 5-methylpyrazin-2-yl;
1-H-imidazol-2-yl, 1-methyl-1H-imidazol-4-yl or 1-methyl-1H-imidazol-2-yl;
benzofuran-2-yl, 5,6-dimethoxybenzofuran-2-yl, or 5-(2-morpholin-4-yl-ethoxy)benzofuran-2-yl;
thiophene-3-yl, or thiophen-2-yl, 5-pyridin-2-ylthiophen-2-yl, 5-methylthiophenyl 3-methylthiophen-2-yl; or 3-ethoxythiophen-2-yl;
furo[3,2-b]-pyridine-2-yl or 3 -methylfuro[3,2-b]pyridin-2-yl;
phenyl, 4-methylphenyl, 3-chlorophenyl, 4-chlorophenyl, 3-trifluoromethylphenyl, 4-trifluoromethylphenyl, 2-chlorophenyl, 4-fluorophenyl, 4-hydroxyphenyl, or 4-acetylphenyl;
pyridin-2-yl; or
thieno[3,2-b]thiophen-2-yl or 5-isoxazol-3-ylthiophen-2-yl;
R⁹ is pyridin-2-yl, phenyl, furan-2-yl or methyl;
R′ is H; and
R″ is H or C₁₋₆alkyl.

10. A compound of Formula II according to claim 7 wherein:
furan-2-carboxylic acid {(S)-2-homocyclohexyl-1-[3-oxo-1-(1-oxy-pyridine-2-sulfonyl)-azepan-4-ylcarbamoyl]-ethyl}-amide;
furan-2-carboxylic acid {(S)-2-tryptophanyl-1-[3-oxo-1-(pyridine-2-sulfonyl)-azepan-4-ylcarbamoyl]-ethyl}-amide;
5-trifluoromethyl-furan-2-carboxylic acid {(S)-2-cyclohexyl-1-[(s)-3-oxo-1-(pyridine-2-sulfonyl)-azepan-4-ylcarbamoyl]-ethyl}-amide;
2,4-dimethyl-thiazole-5-carboxylic acid {(S)-2-cyclohexyl-1-[(s)-3-oxo-1-(pyridine-2-sulfonyl)-azepan-4-ylcarbamoyl]-ethyl}-amide;
5-methyl-pyrazine-2-carboxylic acid {(S)-2-cyclohexyl-1-[(s)-3-oxo-1-(pyridine-2-sulfonyl)-azepan-4-ylcarbamoyl]-ethyl}-amide;
1-methyl-1H-imidazole-2-carboxylic acid {(S)-2-cyclohexyl-1-[(s)-3-oxo-1-(pyridine-2-sulfonyl)-azepan-4-ylcarbamoyl]-ethyl}-amide;
1H-pyrazole-4-carboxylic acid {(S)-2-cyclohexyl-1-[(s)-3-oxo-1-(pyridine-2-sulfonyl)-azepan-4-ylcarbamoyl]-ethyl}-amide;
4-methyl-2-phenyl-thiazole-5-carboxylic acid {(S)-2-cyclohexyl-1-[(s)-3-oxo-1-(pyridine-2-sulfonyl)-azepan-4-ylcarbamoyl]-ethyl}-amide;
2,5-dimethyl-furan-3-carboxylic acid {(S)-2-cyclohexyl-1-[(S)-3-oxo-1-(pyridine-2-sulfonyl)-azepan-4-ylcarbamoyl]-ethyl}-amide;
2-methyl-furan-3-carboxylic acid {(S)-2-cyclohexyl-1-[(S)-3-oxo-1-(pyridine-2-sulfonyl)-azepan-4-ylcarbamoyl]-ethyl}-amide;
isoxazole-5-carboxylic acid {(S)-2-cyclohexyl-1-[(S)-3-oxo-1-(pyridine-2-sulfonyl)-azepan-4-ylcarbamoyl]-ethyl}-amide;
5-methyl-isoxazole-3-carboxylic acid {(S)-2-cyclohexyl-1-[(S)-3-oxo-1-(pyridine-2-sulfonyl)-azepan-4-ylcarbamoyl]-ethyl}-amide;
5-methyl-isoxazole-4-carboxylic acid {(S)-2-cyclohexyl-1-[(S)-3-oxo-1-(pyridine-2-sulfonyl)-azepan-4-ylcarbamoyl]-ethyl}-amide;
3-methyl-isoxazole-4-carboxylic acid {(S)-2-cyclohexyl-1-[(S)-3-oxo-1-(pyridine-2-sulfonyl)-azepan-4-ylcarbamoyl]-ethyl}-amide;
2-methyl-2H-pyrazole-3-carboxylic acid {(S)-2-cyclohexyl-1-[(S)-3-oxo-1-(pyridine-2-sulfonyl)-azepan-4-ylcarbamoyl]-ethyl}-amide;
pyrazine-2-carboxylic acid {(S)-2-cyclohexyl-1-[(S)-3-oxo-1-(pyridine-2-sulfonyl)-azepan-4-ylcarbamoyl]-ethyl}-amide;
thiazole-2-carboxylic acid {(S)-2-cyclohexyl-1-[(S)-3-oxo-1-(pyridine-2-sulfonyl)-azepan-4-ylcarbamoyl]-ethyl}-amide;
2-methyl-thiazole-4-carboxylic acid {(S)-2-cyclohexyl-1-[(S)-3-oxo-1-(pyridine-2-sulfonyl)-azepan-4-ylcarbamoyl]-ethyl}-amide;
(S)-3-cyclohexyl-N-[(S)-3-oxo-1-(pyridine-2-sulfonyl)-azepan-4-yl]-2-(thiophene-2-sulfonylamino)-propionamide;
(S)-3-cyclohexyl-2-(1-methyl-1H-imdiazole-4-sulfonylamino)-N-[(S)-3-oxo-1-(pyridine-2-sulfonyl)-azepan-4-yl]-propionamide;
(S)-3-cyclohexyl-2-(3,5-dimethyl-isoxazole-4-sulfonylamino)-N-[(S)-3-oxo-1-(pyridine-2-sulfonyl)-azepan-4-yl]-propionamide;
(S)-3-cyclohexyl-2-(furan-2-sulfonylamino)-N-[(S)-3-oxo-1-(pyridine-2-sulfonyl)-azepan-4-yl]-propionamide;
(S)-3-cyclohexyl-N-[(S)-3-oxo-1-(pyridine-2-sulfonyl)-azepan-4-yl]-2-(pyridine-2-sulfonylamino)-propionamide;
(S)-3-cyclohexyl-2-(morpholine-4-sulfonylamino)-N-[(S)-3-oxo-1-(pyridine-2-sulfonyl)-azepan-4-yl]-propionamide;
(S) -3-cyclohexyl-2-(5-isoxazol-3-yl-thiophene-2-sulfonylamino)-N-[(S)-3-oxo-1-(pyridine-2-sulfonyl)-azepan-4-yl]-propionamide;
4-{(S)-3-cyclopentyl-2-[(1-furan-2-yl-methanoyl)-amino]-propanoylamino}-3-oxo-azepane-1-carboxylic acid benzyl ester;
furan-2-carboxylic acid {(S)-2-cyclopentyl-1-[3-oxo-1-(pyridine-2-sulfonyl)-azepan-4-ylcarbamoyl]ethyl}-amide;
furan-2-carboxylic acid [(S)-2-cyclopentyl-1-(1-methanesulfonyl-3-oxo-azepan-4-ylcarbamoyl)-ethyl]-amide;
furan-2-carboxylic acid [(S)-1-(1-benzenesulfonyl-3-oxo-azepan-4-ylcarbamoyl)-2-cyclopentyl-ethyl]-amide
furan-2-carboxylic acid {(S)-2-cyclopentyl-1-[1-(1-furan-2-yl-methanoyl)-3-oxo-azepan-4-ylcarbamoyl]-ethyl}-amide;
furan-2-carboxylic acid {(S)-2-cyclopentyl-1-[3-oxo-1-(1-phenyl-methanoyl)-azepan-4-ylcarbamoyl]-ethyl}-amide; or
piperazine-1-carboxylic acid {(S)-2-cyclopentyl-1-[(4S,7R)-7-methyl-3-oxo-1-(pyridine-2-sulfonyl)-azepan-4-ylcarbamoyl]-ethyl}-amide; or
a pharmaceutically acceptable salt, hydrate or solvate thereof.
